(19) United States
(12) Patent Application Publication
(10) Pub. No.: US 2026/0002176 A1
(43) Pub. Date: Jan. 1, 2026

(54) ADENO-ASSOCIATED VIRUS GENE THERAPY PRODUCTS AND METHODS

(71) Applicant: The Research Institute at Nationwide Children's Hospital, Columbus, OH (US)

(72) Inventors: Brian K. Kaspar, Westerville, OH (US); Kathrin Christine Meyer, columbus, OH (US); Shibi Likhite, Columbus, OH (US)

(21) Appl. No.: 18/880,819

(22) PCT Filed: Jul. 11, 2023
(86) PCT No.: PCT/US23/69966

§ 371 Date: Jan. 2, 2025

Related U.S. Application Data
(60) Provisional application No. 63/388,492, filed on Jul. 12, 2022.

(51) Int. Cl. C12N 15/86; A61K 48/00; A61P 25/00; A61P 25/28; C07K 16/28; C12N 15/113

(52) U.S. Cl. CPC C12N 15/86; A61K 48/0075; A61P 25/00; A61P 25/28; C07K 16/2851; C12N 15/1137; C12Y 115/01001; C12N 2310/531; C12N 2750/14122; C12N 2750/14143; C12N 2750/14152

(57) ABSTRACT

Adeno-associated virus (AAV) gene therapy vectors express a therapeutic protein or RNA that treats a genetic defect in a cell. The present disclosure provides AAV gene therapy vectors that additionally express an anti-inflammatory protein or peptide. When the provided AAV gene therapy vectors are used in methods of treatment of. for example. neurodegenerative diseases. the therapeutic protein/RNA treats the genetic defect within the cells directly transduced by the AAV vectors while the anti-inflammatory protein/peptide is secreted by the transduced cells into the intercellular milieu and treats microglial activation associated with the neuroinflammatory component of the neurodegenerative diseases. The therapeutic protein and anti-inflammatory protein/peptide can be expressed as fusion protein in which the two are separated in the fusion protein by a self-cleaving peptide. The provided AAV gene therapy vectors and methods are thus useful in treating neurological and neurodegenerative disorders.

### CROSS-REFERENCE TO RELATED APPLICATIONS

This application claims priority to U.S. Provisional Application No. 63/388,492, filed Jul. 12, 2022, which is incorporated by reference herein in its entirety.

### INCORPORATION BY REFERENCE OF THE SEQUENCE LISTING

This application contains, as a separate part of disclosure, a Sequence Listing in computer-readable form (Filename: 58161_SeqListing.xml; 304,231 bytes—XML file dated Jul. 11, 2023) which is incorporated by reference herein in its entirety.

### FIELD

Adeno-associated virus (AAV) gene therapy vectors express a therapeutic protein or RNA that treats a genetic defect in a cell. The present disclosure provides AAV gene therapy vectors that additionally express an anti-inflammatory protein or peptide. When the provided AAV gene therapy vectors are used in methods of treatment of, for example, neurodegenerative diseases, the therapeutic protein/RNA treats the genetic defect within the cells directly transduced by the AAV vectors while the anti-inflammatory protein/peptide is secreted by the transduced cells into the intercellular milieu and treats microglial activation associated with the neuroinflammatory component of the neurodegenerative diseases. From the AAV gene therapy vector, the therapeutic protein and anti-inflammatory protein/peptide can be expressed separately or as a fusion protein in which the two are separated in the fusion protein by a self-cleaving peptide. The anti-inflammatory protein/peptide can alternatively be expressed from a separate vector from the AAV gene therapy vector. The provided AAV gene therapy vectors and methods are thus useful in treating neurological and neurodegenerative disorders such as Amyotrophic Lateral Sclerosis, Duchenne's Muscular Dystrophy), Spinal Muscular Atrophy, Batten diseases (CLN1/3/6/8), IGHMBP2-related disorder, Pitt-Hopkins Syndrome and PGAP3 Congenital Disorder of Glycosylation, including the neuroinflammation associated with the disorders.

### BACKGROUND

Neurodegeneration involves the loss of neuronal function and structure. Neuroinflammation is associated with neurodegeneration in neurodegenerative diseases. Neuroinflammation is characterized by the activation of astrocytes as well as microglia, the neuroimmune cells of the central nervous system [Hernández et al., Glycoconjugate Journal, https://doi.org/10.1007/s10719-022-10064-2 (published online Jun. 2, 2022)].

Amyotrophic Lateral Sclerosis (ALS) is an example of a devastating neurodegenerative disease characterized by motor neuron degeneration, leading to progressive paralysis and death. As no cure has been identified, most patients die within 2-5 years of symptom onset. ALS is the most common adult-onset motor neuron disease with an incidence of 2:100,000 and, due to an increasing aging population, the number of patients in developed and developing countries is projected to rise over 30% by the year 2040. Although several therapeutics have been approved to ameliorate this condition, treatments that truly halt disease progression are lacking likely due to the non-cell autonomous nature of ALS pathogenesis. The four FDA approved therapies for ALS extend survival of a subpopulation of patients by a few months, but do not significantly improve muscle strength or quality of life. As patients continue to decline, frequent hospital visits and expensive equipment, as well as specialized patient care, are required.

Though the cause of ALS remains largely unknown, it has been determined that 20% of familial inherited ALS cases are caused by gain of function mutations in the superoxide dismutase 1 (SOD1) gene. Superoxide dismutase is an enzyme that breaks down harmful free superoxide radicals, thereby eliminating free radical induced oxidative stress. Misfolded, non-mutated SOD1 might additionally play a role in an even larger ALS subpopulation. An adeno-associated viral vector serotype 9 (AAV9) mediated gene therapy using a small hairpin RNA (shRNA) has been used to reduce the abundance of the toxic SOD1 protein. The shRNA expressed by this AAV9 leads to the degradation of SOD1 mRNA, resulting in the knockdown of SOD1 protein. Suppression of SOD1 levels extended survival and preserved motor function in two different ALS mouse models (SOD 1G93A and SOD1^{G37R} mice), but mice treated with AAV9.SOD1.shRNA still succumb to the disease, albeit at a later time point [Foust et al., Mol. Ther., 21:2148-59 (2013); Bravo-Hernandez, et al., Nat. Med, 26:118-130 (2020); Iannitti et al., Mol. Ther. Nucleic Acid, 12:75-88 (2018)].

Research groups have demonstrated the impact of non-neuronal cells (such as astrocytes, oligodendrocytes, and microglia) on motor neuron death in ALS and have shown that individual correction of each cell type has beneficial effects on the disease outcome [Di Giorgio et al., Nat. Neurosci., 10:608-614 (2007); Nagai et al., Nat. Neurosci., 10:615-622 (2007); Marchetto et al., Cell Stem Cell, 3:649-657 (2008); Ilieva and Cleveland, Journal of Cell Biology, 187:761-772 (2009); Lobsiger et al., Proc. Natl. Acad. Sci. U. S. A., 106:4465-4470 (2009); Haidet-Phillips et al., Nat. Biotechnol., 29:824-828 (2011); Meyer, Proc. Natl. Acad. Sci. U.S.A., 111:829-32 (2014); Madill et al., Mol. Brain, 10:22 (2017): Meyer and Kaspar, Brain Res., 1656:27-39 (2017); Serio and Patani, Stem Cells, 36:293-303 (2018); Beckman et al., Trends Neurosci, 24 (11 Suppl.): S15-20 (2001)].

In ALS, microglia become chronically activated and neurotoxic, rapidly altering their transcription profiles, and releasing proinflammatory cytokines and chemokines. These inflammatory microglia also undergo cytoskeletal rearrangements which change surface receptor expression and allow for migration to sites of neurodegeneration. Though the exact mechanism of microglial activation is unclear, it is known that mutant SOD1 expressed in microglia is a microglial activator, thereby propagating degeneration of motor neurons [Massenzio et al., Biochim. Biophys. Acta—Mol. Basis Dis., 1864:3771-3785 (2018)].

Transgenic reduction of microglial activation has a beneficial effect in a mouse model, model SOD 1^{G93A} mice [Martínez-Muriana et al., Sci. Rep. 6:25663 (2016); Zhao et al., Journal of Neuropathology and Experimental Neurology, 63 (9): 964-977 (2004); Xiao et al., J. Neurochem 102:2008-2019 (2007); Weydt et al., Glia, 48 (2): 179-182 (2004); Frakes et al., Neuron 81:1009-1023 (2014); Boillée et al., Science, 312:1389-92 (2006); Liu and Wang, Frontiers in Immunology, 8: Article 1005 (2017)]. This effect is further enhanced by combination with AAV9.SOD1.shRNA [Frakes et al. (2014), supra; Frakes, Ann. Clin. Transl. Neurol., 4:76-86 (2017). See also, the experiments discussed in Kato et al., Current Drug Targets, 6:407-418 (2005) and Chang-Hong et al., Experimental Neurology, 194:203-211 (2005) of injection of recombinant galectin-1 into muscle of transgenic mice with the ALS-linked SOD1 mutation H46R. However, treatments targeting this cell type alone have shown disappointing results in clinical trials [https://alsnewstoday.com/2018/04/27/np001-fails-improve-als-disease-severity-pulmonary-function-phase-2-trial/].

There remains a need in the art for products and methods for treating neurodegenerative disorders such as ALS, and in particular for additionally treating the neuroinflammatory component of such disorders.

### SUMMARY

In post-mortem brain and spinal cord tissue in end stage SOD1^{G93A} mice, very intense microglial inflammation is observed, a finding also observed in post-mortem ALS patient spinal cords. Contemplating microglia are a critical cell type influencing ALS progression, methods disclosed herein target this cell type for an optimal and potentially curative effect with any ALS treatment regimen. The methods focus on a combination therapeutic approach, gene therapy to directly target neurons and astrocytes, combined with treatments that target microglia indirectly. In particular, microglia are targeted herein by, for example, the addition of a Galectin-1 (GAL1) expression cassette to an AAV9.SOD1.shRNA gene therapy vector. GAL1 is a secreted protein that can act in trans on microglia. Neurons and astrocytes directly transduced with the AAV9 expressing GAL1 overexpress and continuously secrete GAL1, which acts on adjacent microglia at sites of inflammation thereby signaling to the microglia to remain in a non-inflamed state. Since microglia are not eliminated with GAL1 treatment, this approach preserves the beneficial effects of non-inflammatory microglia.

The disclosure contemplates microglial inflammation is a crucial aspect of most neurodegenerative disorders, and contemplates a parallel combination approach can improve gene therapy strategies for a broad variety of neurological disorders other than ALS such as Duchenne's Muscular Dystrophy (DMD), spinal muscular atrophy (SMA), Batten diseases (CLN1/3/6/8), IGHMBP2-related disorder, Pitt-Hopkins Syndrome and PGAP3 Congenital Disorder of Glycosylation.

The disclosure provides a recombinant adeno-associated virus (rAAV) genome that expresses (A) a therapeutic protein or RNA for gene therapy and (B) an anti-inflammatory protein or peptide. The disclosure also provides rAAV genomes that express only an anti-inflammatory protein or peptide.

The rAAV genome can be a rAAV genome wherein (A) is a short hairpin ribonucleic acid targeting superoxide dismutase 1 (SOD1 shRNA) and (B) is a galectin, a metallothionein protein, a metallothionein fusion protein, NBD 1X or NBD 3X. The sequence of the SOD1 shRNA can be SEQ ID NO: 4. The galectin can be human Galectin-1 or human Galectin-3. The rAAV genome can be a rAAV genome wherein (B) is NBD 1X or NBD 3X. The rAAV genome can be a rAAV genome wherein (B) is a metallothionein protein, a metallothionein fusion protein, NBD 1X or NBD 3X. The expression of (A) can be under the control of an H1 promoter. The expression of (B) can be under the control of a CBA promoter.

The disclosure provides rAAV comprising the genomes provided. The rAAV can be a scAAV. The rAAV can be a ssAAV. The rAAV can comprise AAV9 capsid.

The disclosure provides compositions comprising the rAAV provided herein. The compositions can be formulated for administration to a subject (e.g., a human patient) direct injection into the cerebrospinal fluid, intracerebroventricular delivery, intrathecal delivery or intravenous delivery.

The compositions can comprise an agent that increases the viscosity or density of the composition, such as a contrast agent.

The disclosure provides methods of treating a neurological or neurodegenerative disorder (such as ALS, DMD, SMA, Batten diseases (CLN1/3/6/8), an IGHMBP2-related disorder, Pitt-Hopkins Syndrome and PGAP3 Congenital Disorder of Glycosylation) in a subject comprising administering to the subject an effective amount of an rAAV composition provided herein. The rAAV compositions can be administered to the subject by direct injection into the cerebrospinal fluid, intracerebroventricular delivery, intrathecal delivery or intravenous delivery. The methods of treating diseases (including, but not limited to, ALS, Batten disease, a IGHMBP2-related disorder, Pitt-Hopkins Syndrome or PGAP3 Congenital Disorder of Glycosylation) in a subject comprise administering to the subject an effective amount of an rAAV composition expressing: for ALS a SOD1 shRNA, for Batten disease a CLN1, CLN3, CLN6 or CLN8 protein, for an IGHMBP2-related disorder a IGHMBP2 protein or for a PGAP3 Congenital Disorder of Glycosylation a PGAP3 protein.

The disclosure provides plasmids comprising the rAAV genomes provided herein, as well as methods of producing rAAV by transforming/transfecting packaging cells with the plasmids and culturing the packaging cells.

### BRIEF DESCRIPTION OF THE DRAWINGS

FIG. 1 shows Galectin-1 conditioning of SOD1^{G93A} microglia rescues the motor neuron toxicity in vitro.

FIG. 2 shows extracellular Galectin-1 reduces secretion of TNF-α from SOD1^{G93A} microglia.

FIG. 3 shows Galectin-1 conditioning reduces the expression of M1 markers and enhances the expression of anti-inflammatory M2 markers in SOD1^{G93A} microglia.

FIG. 4 shows Galectin-1 conditioning reduces the pro-inflammatory NF-κB activation in co-cultured SOD1^{G93A} microglia.

FIG. 5 shows the design of experiments in mice. B6SJL-Tg(SOD1*G93A)1Gur/J ×C57BL/6J mice received shRNA and/or GAL1.

FIG. 6 shows a plasmid map and plasmid sequence (SEQ ID NO: 43) for producing an scAAV that expresses murine Galectin-1.

FIG. 7 shows a plasmid map and plasmid sequence (SEQ ID NO: 44) for producing an scAAV (scAAV.SOD1.shRNA.msGal1) that expresses a SOD1 shRNA and murine Galectin-1.

FIG. 8 shows scAAV.SOD1.shRNA.msGal1 reduces SOD1 expression and increases Gal1 expression in transfected HEK293s.

FIG. 9 shows scAAV9.SOD1.shRNA.Gal1 increases Gal1 expression in WT/SOD1^{G93A} mice and reduces SOD1 expression in SOD1^{G93A} mice.

FIG. 10 shows intracerebroventricular delivery of AAV9.SOD1.shRNA.msGal1 improves survival in SOD1^{G93A} mice over AAV9.SOD1.shRNA treatment alone.

FIG. 11 shows intracerebroventricular delivery of AAV9.SOD1.shRNA.msGal1 improves survival in SOD1^{G93A} male mice over AAV9.SOD1.shRNA treatment alone.

FIG. 12 shows intracerebroventricular delivery of AAV9.SOD1.shRNA.msGal1

improves survival in SOD1^{G93A} female mice over AAV9.SOD1.shRNA treatment alone.

FIG. 13 shows intracerebroventricular delivery of AAV9.SOD1.shRNA.msGal1 and AAV9.SOD1.shRNA improves motor performance in SOD1^{G93A} mice.

FIG. 14 shows intrathecal delivery of PHP.B.SOD1.shRNA.msGal1 improves survival in SOD1^{G93A} mice.

FIG. 15 shows intrathecal delivery of PHP.B.SOD1.shRNA.msGal1 improves body weight in SOD1^{G93A} mice.

FIG. 16 shows a plasmid map and plasmid sequence (SEQ ID NO: 45) for producing an scAAV (scAAV.SOD1.shRNA.hGal1) that expresses a SOD1 shRNA and human Galectin-1.

FIG. 17 shows a plasmid map and plasmid sequence (SEQ ID NO: 46) for producing an scAAV (scAAV.SOD1.hGal1) that expresses human Galectin-1.

FIG. 18 sets out DNA (SEQ ID NO: 35) and amino acid (SEQ ID NO: 36) sequences of the NBD peptide (“NBD 1X peptide”) and DNA (SEQ ID NO: 37) and amino acid (SEQ ID NO: 38) sequences of an auto-cleaving fusion protein comprising three copies of the NBD peptide (“NBD 3X peptide”).

FIG. 19 shows AAV9.Gal1 treatment of TCF4 iAstrocytes improves neuronal survival.

FIG. 20 shows CLN3 iNeuron phenotype is improved by treatment with AAV9.Gal1.

FIG. 21 shows expression of Galectin-1 from fusion constructs.

FIG. 22 shows PGAP3 iAstrocyte toxicity improves with gene therapy treatment.

FIG. 23 shows a plasmid map and plasmid sequence (SEQ ID NO: 47) for producing an scAAV (scAAV.P546.CLN1.Gal1) that expresses a human CLN1 and human Galectin-1.

FIG. 24 shows a plasmid map and plasmid sequence (SEQ ID NO: 48) for producing an scAAV (scAAV.CB.CLN1.Gal1) that expresses a human CLN1 and human Galectin-1.

FIG. 25 shows a plasmid map and plasmid sequence (SEQ ID NO: 51) for producing an scAAV (scAAV.P546.CLN3.Gal1) that expresses a human CLN3 and human Galectin-1.

FIG. 26 shows a plasmid map and plasmid sequence (SEQ ID NO: 52) for producing an scAAV (scAAV.CB.CLN3.Gal1) that expresses a human CLN3 and human Galectin-1.

FIG. 27 shows a plasmid map and plasmid sequence (SEQ ID NO: 55) for producing an scAAV (scAAV.CB.CLN6.Gal1) that expresses a human CLN6 and human Galectin-1.

FIG. 28 shows a plasmid map and plasmid sequence (SEQ ID NO: 56) for producing an scAAV (scAAV.CB.CLN6.Gal1) that expresses a human CLN6 and human Galectin-1.

FIG. 29 shows a plasmid map and plasmid sequence (SEQ ID NO: 59) for producing an scAAV (scAAV.P546.CLN8.Gal1) that expresses a human CLN8 and human Galectin-1.

FIG. 30 shows a plasmid map and plasmid sequence (SEQ ID NO: 60) for producing an scAAV (scAAV.CB.CLN8.Gal1) that expresses a human CLN8 and human Galectin-1.

FIG. 31 shows a plasmid map and plasmid sequence (SEQ ID NO: 63) for producing an scAAV (scAAV.P546.IGHMBP2.Gal1) that expresses a IGHMBP2and human Galectin-1.

FIG. 32 shows a plasmid map and plasmid sequence (SEQ ID NO: 64) for producing an scAAV (scAAV.CB.IGHMBP2.Gal1) that expresses a IGHMBP2 and human Galectin-1.

FIG. 33 shows a plasmid map and plasmid sequence (SEQ ID NO: 67) for producing an scAAV (scAAV.546.PGAP3.Gal1) that expresses a PGAP3 and human Galectin-1.

FIG. 34 shows a plasmid map and plasmid sequence (SEQ ID NO: 68) for producing an scAAV (scAAV.CBA.PGAP3.Gal1) that expresses a PGAP3 and human Galectin-1.

FIG. 35 shows a plasmid map and plasmid sequence (SEQ ID NO: 71) for producing an scAAV (scAAV.shSOD1.msGal1.MT1X) that expresses a SOD1 shRNA and a murine Galectin-1 metallothionine fusion protein.

FIG. 36 shows plasmid map and plasmid sequence (SEQ ID NO: 74) for producing an scAAV (scAAV.shSOD1.hGal1.MT1X) that expresses a SOD1 shRNA and a human Galectin-1 metallothionine fusion protein.

FIG. 37 shows a plasmid map and plasmid sequence (SEQ ID NO: 78) for producing an scAAV (scAAV.P546.3xMT1X) that expresses a 3x metallothionine fusion protein.

FIG. 38 shows another plasmid map and plasmid sequence (SEQ ID NO: 79) for producing an scAAV (scAAV.CB.3xMT1X) that expresses a 3x metallothionine fusion protein.

### DETAILED DESCRIPTION

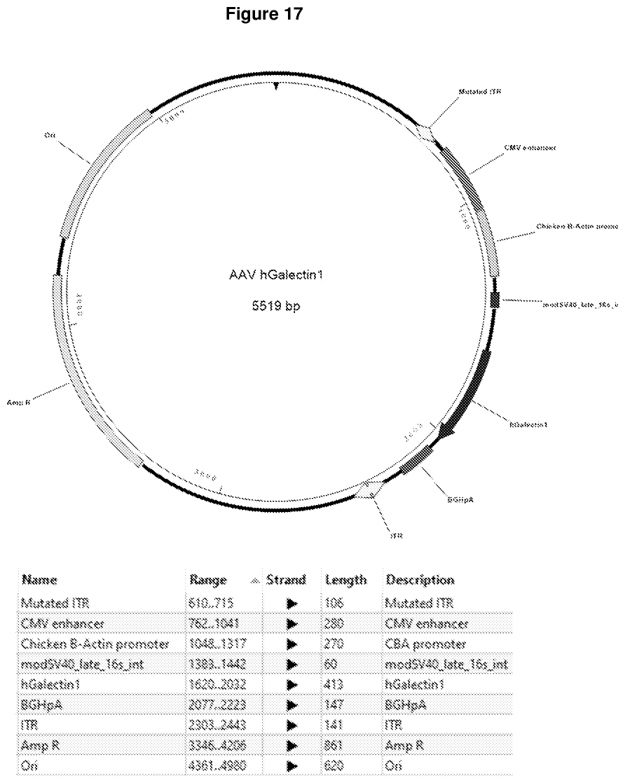

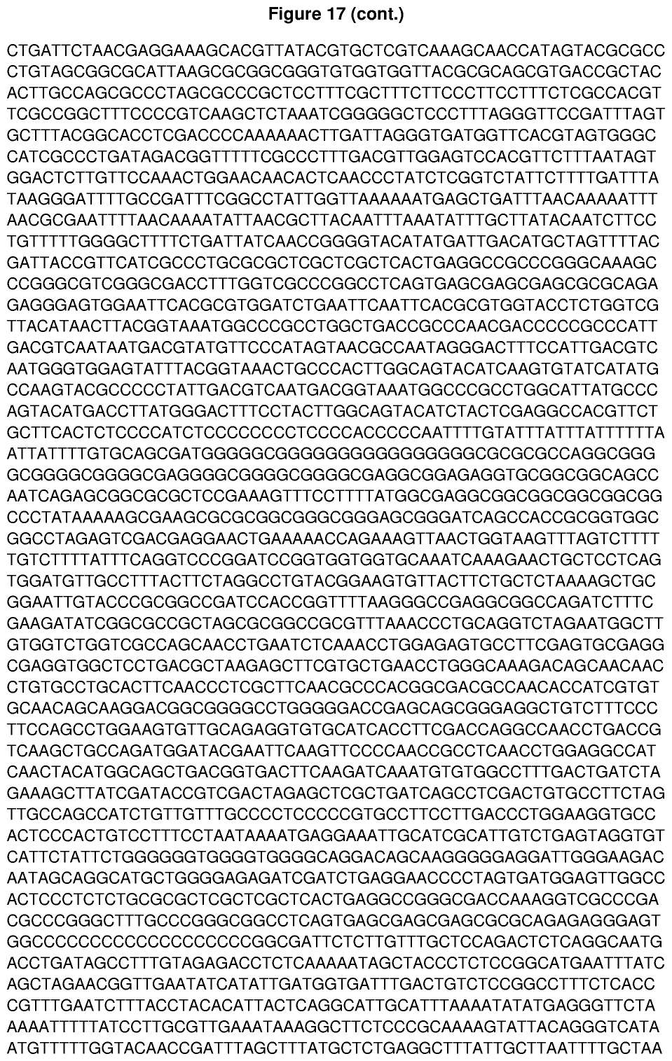

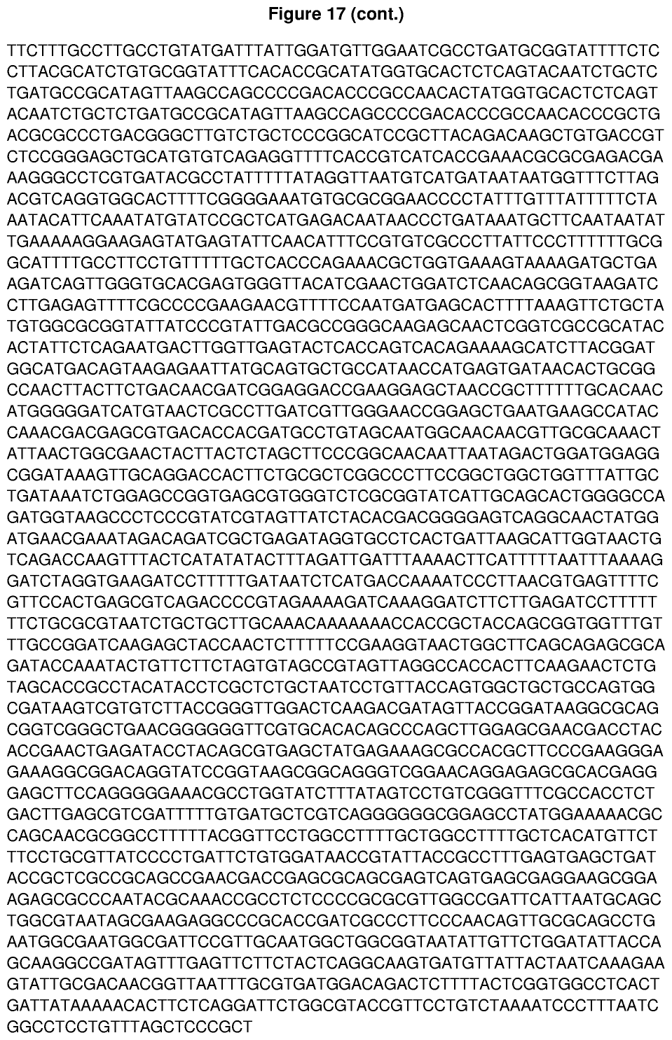

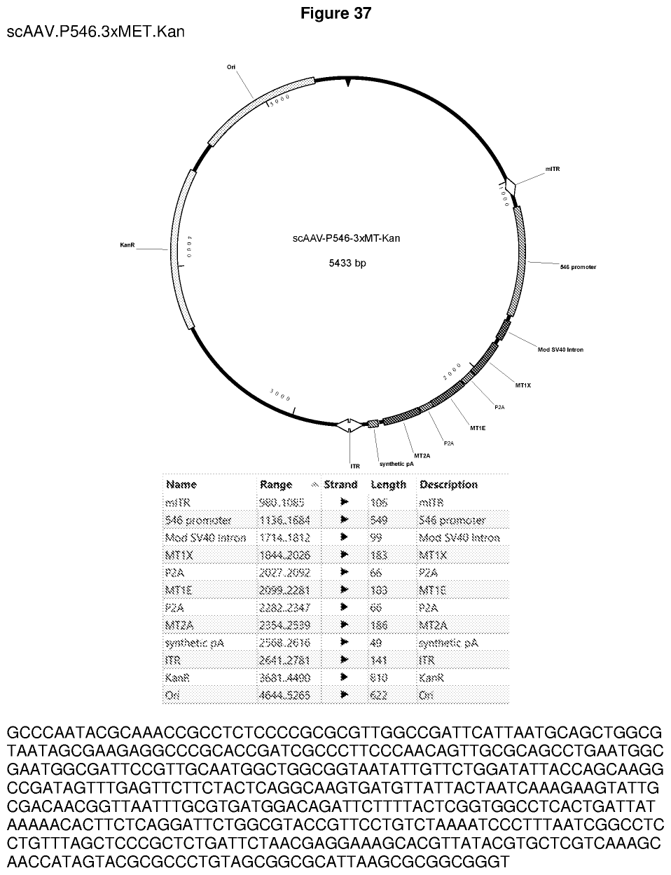

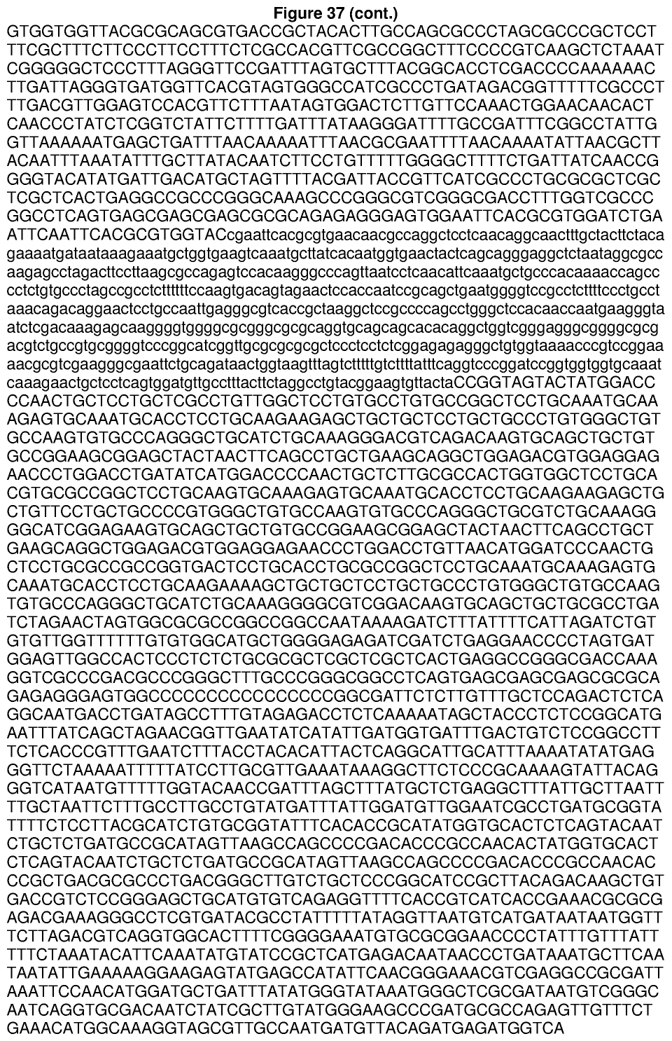

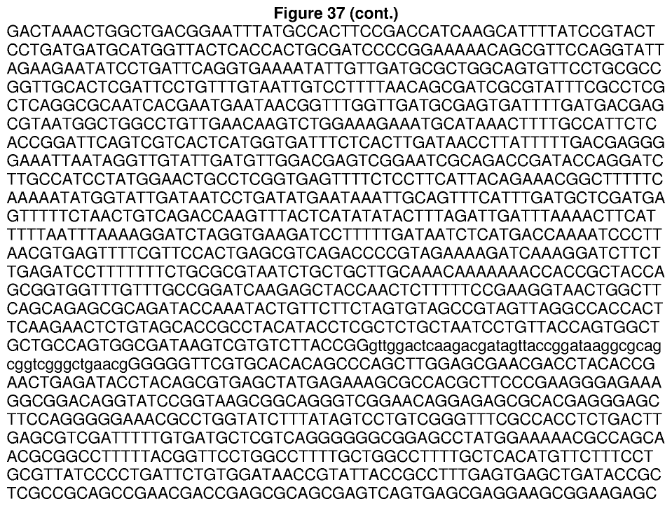

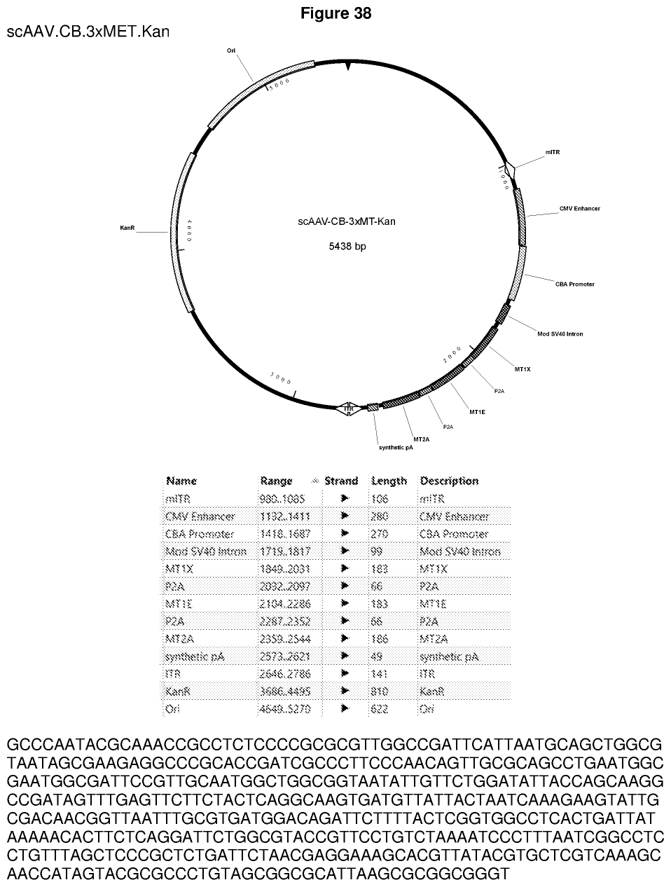

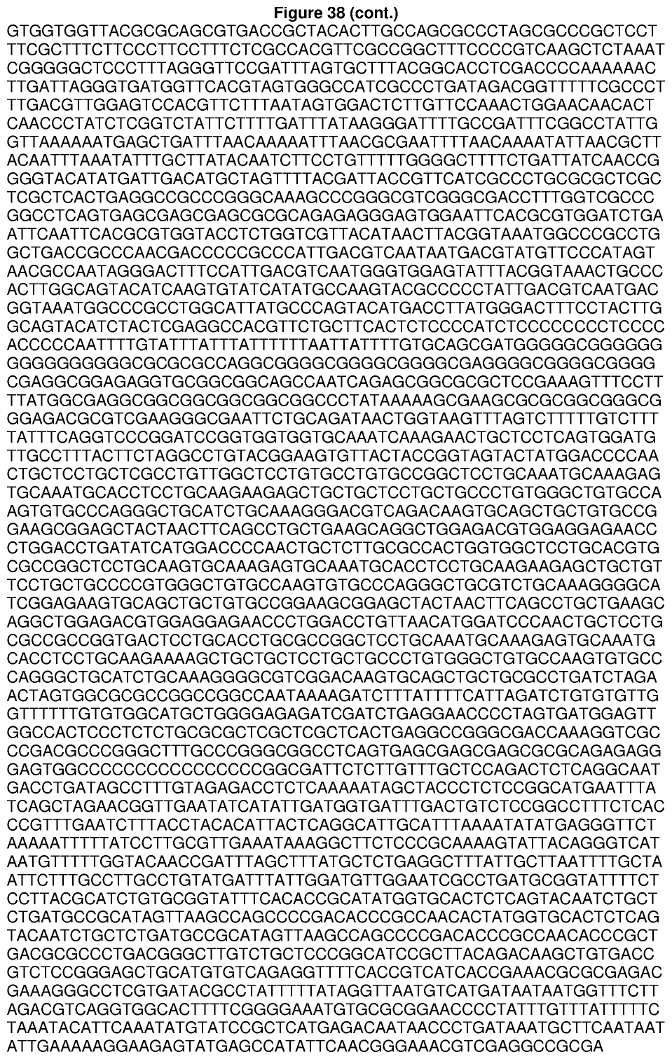

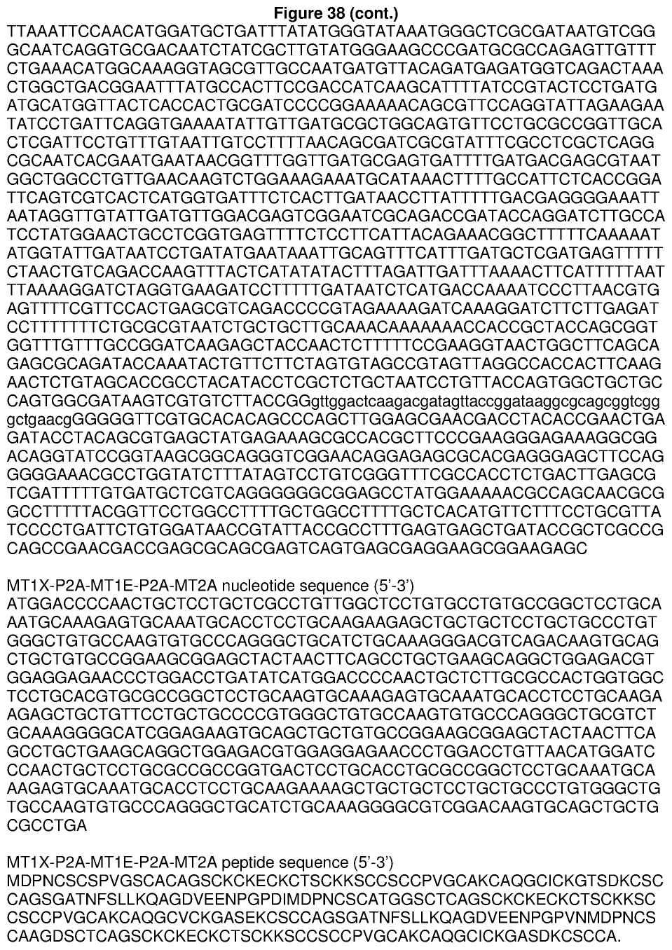

The disclosure provides AAV with genomes comprising one or more AAV ITRs flanking expression cassettes encoding (1) a therapeutic protein or peptide and (2) an anti-inflammatory protein or peptide. The disclosure also provides separate vectors expressing an anti-inflammatory protein or peptide (including, but not limited to, rAAV produced using the plasmid shown in FIGS. 17, 37 and 38).

For example, the disclosure provides AAV gene therapy vectors with genomes expressing (1) one or more RNAs (including, but not limited to, small hairpin RNAs, antisense RNAs and/or microRNAs) that target mutant SOD1 polynucleotides and (2) a galectin. The disclosure also provides AAV vectors expressing a galectin (including, but not limited to, rAAV produced using the plasmid shown in FIG. 17). The examples describe the use of exemplary rAAV encoding small hairpin RNAs (shRNAs) and a galectin. In the rAAV genomes, shRNA-encoding DNA and galectin-encoding DNA are each operatively linked to transcriptional control DNA, specifically promoter DNA that is functional in target cells, to form expression cassettes. The rAAV genome can comprise an expression cassette encoding a SOD1 shRNA such as:

(SEQ ID NO: 1)
GCATCATCAATTTCGAGCAGAAGGAA,
(SEQ ID NO: 2)
GAAGCATTAAAGGACTGACTGAA,
(SEQ ID NO: 3)
CTGACTGAAGGCCTGCATGGATT,
(SEQ ID NO: 4)
CATGGATTCCATGTTCATGA,
(SEQ ID NO: 5)
GCATGGATTCCATGTTCATGA,
(SEQ ID NO: 6)
GGTCTGGCCTATAAAGTAGTC,
(SEQ ID NO: 7)
GGGCATCATCAATTTCGAGCA,
(SEQ ID NO: 8)
GCATCATCAATTTCGAGCAGA,
(SEQ ID NO: 9)
GCCTGCATGGATTCCATGTTC,
(SEQ ID NO: 10)
GGAGGTCTGGCCTATAAAGTA,
(SEQ ID NO: 11)
GATTCCATGTTCATGAGTTTG,
(SEQ ID NO: 12)
GGAGATAATACAGCAGGCTGT,
(SEQ ID NO: 13)
GCTTTAAAGTACCTGTAGTGA,
(SEQ ID NO: 14)
GCATTAAAGGACTGACTGAAG,
(SEQ ID NO: 15)
TCATCAATTTCGAGCAGAA,
(SEQ ID NO: 16)
TCGAGCAGAAGGAAAGTAA,
(SEQ ID NO: 17)
GCCTGCATGGATTCCATGT,
(SEQ ID NO: 18)
TCACTCTCAGGAGACCATT,
or
(SEQ ID NO: 19)
GCTTTAAAGTACCTGTAGT.

Commercial providers such as Ambion Inc. (Austin, TX), Darmacon Inc. (Lafayette, CO), InvivoGen (San Diego, CA), and Molecular Research Laboratories, LLC (Herndon, VA) generate custom inhibitory RNA molecules. In addition, commercially kits are available to produce custom siRNA molecules, such as SILENCER™ siRNA Construction Kit (Ambion Inc., Austin, TX) or psiRNA System (InvivoGen, San Diego, CA).

Galectins, soluble β-galactoside-binding proteins, are widely expressed at sites of inflammation and play an active role in amplification or resolution of inflammatory responses [Sundblad et al., J. Immunol. 199:3721-3730 (2017)].

Galectin-1 (GAL1), a glycan-binding protein, counteracts the synthesis of pro-inflammatory cytokines, displaying broad anti-inflammatory properties. GAL1 has been shown to attenuate microglial activation by shifting neurotoxic microglia to a neuroprotective M2 phenotype [Starossom et al., Immunity, 37:249-263 (2012)].

A human GAL1 DNA sequence is set out below.
(SEQ ID NO: 20)
5′ATGGCTTGTGGTCTGGTCGCCAGCAACCTGAATCTCAAACCTGGAGAG
TGCCTTCGAGTGCGAGGCGAGGTGGCTCCTGACGCTAAGAGCTTCGTGCT
GAACCTGGGCAAAGACAGCAACAACCTGTGCCTGCACTTCAACCCTCGCT
TCAACGCCCACGGCGACGCCAACACCATCGTGTGCAACAGCAAGGACGGC
GGGGCCTGGGGGACCGAGCAGCGGGAGGCTGTCTTTCCCTTCCAGCCTGG
AAGTGTTGCAGAGGTGTGCATCACCTTCGACCAGGCCAACCTGACCGTCA
AGCTGCCAGATGGATACGAATTCAAGTTCCCCAACCGCCTCAACCTGGAG
GCCATCAACTACATGGCAGCTGACGGTGACTTCAAGATCAAATGTGTGGC
CTTTGACTGA3′
A mouse GAL1 DNA sequence is set out below.
(SEQ ID NO: 21)
5′ATGGCCTGTGGTCTGGTCGCCAGCAACCTGAATCTCAAACCTGGGGAA
TGTCTCAAAGTTCGGGGAGAGGTGGCCTCGGACGCCAAGAGCTTTGTGCT
GAACCTGGGAAAAGACAGCAACAACCTGTGCCTACACTTCAATCCTCGCT
TCAATGCCCATGGAGACGCCAACACCATTGTGTGTAACACCAAGGAAGAT
GGGACCTGGGGAACCGAACACCGGGAACCTGCCTTCCCCTTCCAGCCCGG
GAGCATCACAGAGGTGTGCATCACCTTTGACCAGGCTGACCTGACCATCA
AGCTGCCAGACGGACATGAATTCAAGTTCCCCAACCGCCTCAACATGGAG
GCCATCAACTACATGGGGGGGGATGGAGACTTCAAGATTAAGTGCGTGGC
CTTTGA3′
Galectin-3 also plays a role in neuroinflammation
in chronic neurogenerative diseases [Lerman et
al., Brain and Behavior, 2(5): 563-575 (2012)].
A human Galectin-3 DNA sequence is set out below.
(SEQ ID NO: 22)
5′ATGGCAGACAATTTTTCGCTCCATGATGCGTTATCTGGGTCTGGAAAC
CCAAACCCTCAAGGATGGCCTGGCGCATGGGGGAACCAGCCTGCTGGGGC
AGGGGGCTACCCAGGGGCTTCCTATCCTGGGGCCTACCCCGGGCAGGCAC
CCCCAGGGGCTTATCCTGGACAGGCACCTCCAGGCGCCTACCCTGGAGCA
CCTGGAGCTTATCCCGGAGCACCTGCACCTGGAGTCTACCCAGGGCCACC
CAGCGGCCCTGGGGCCTACCCATCTTCTGGACAGCCAAGTGCCACCGGAG
CCTACCCTGCCACTGGCCCCTATGGCGCCCCTGCTGGGCCACTGATTGTG
CCTTATAACCTGCCTTTGCCTGGGGGAGTGGTGCCTCGCATGCTGATAAC
AATTCTGGGCACGGTGAAGCCCAATGCAAACAGAATTGCTTTAGATTTCC
AAAGAGGGAATGATGTTGCCTTCCACTTTAACCCACGCTTCAATGAGAAC
AACAGGAGAGTCATTGTTTGCAATACAAAGCTGGATAATAACTGGGGAAG
GGAAGAAAGACAGTCGGTTTTCCCATTTGAAAGTGGGAAACCATTCAAAA
TACAAGTACTGGTTGAACCTGACCACTTCAAGGTTGCAGTGAATGATGCT
CACTTGTTGCAGTACAATCATCGGGTTAAAAAACTCAATGAAATCAGCAA
ACTGGGAATTTCTGGTGACATAGACCTCACCAGTGCTTCATATACCATGA
TATAA3′
A mouse Galectin-3 DNA sequence is set out below.
(SEQ ID NO: 23)
5′ATGGCAGACAGCTTTTCGCTTAACGATGCCTTAGCTGGCTCTGGAAAC
CCAAACCCTCAAGGATATCCGGGTGCATGGGGGAACCAGCCTGGGGCAGG
GGGCTACCCAGGGGCTGCTTATCCTGGGGCCTACCCAGGACAAGCTCCTC
CAGGGGCCTACCCAGGACAGGCTCCTCCAGGGGCCTACCCAGGACAGGCT
CCTCCTAGTGCCTACCCCGGCCCAACTGCCCCTGGAGCTTATCCTGGCCC
AACTGCCCCTGGAGCTTATCCTGGCTCAACTGCCCCTGGAGCCTTCCCAG
GGCAACCTGGGGCACCTGGGGCCTACCCCAGTGCTCCTGGAGGCTATCCT
GCTGCTGGCCCTTATGGTGTCCCCGCTGGACCACTGACGGTGCCCTATGA
CCTGCCCTTGCCTGGAGGAGTCATGCCCCGCATGCTGATCACAATCATGG
GCACAGTGAAACCCAACGCAAACAGGATTGTTCTAGATTTCAGGAGAGGG
AATGATGTTGCCTTCCACTTTAACCCCCGCTTCAATGAGAACAACAGGAG
AGTCATTGTGTGTAACACGAAGCAGGACAATAACTGGGGAAAGGAAGAAA
GACAGTCAGCCTTCCCCTTTGAGAGTGGCAAACCATTCAAAATACAAGTC
CTGGTTGAAGCTGACCACTTCAAGGTTGCGGTCAACGATGCTCACCTACT
GCAGTACAACCATCGGATGAAGAACCTCCGGGAAATCAGCCAACTGGGGA
TCAGTGGTGACATAACCCTCACCAGCGCTAACCACGCCATGATCTAA3′

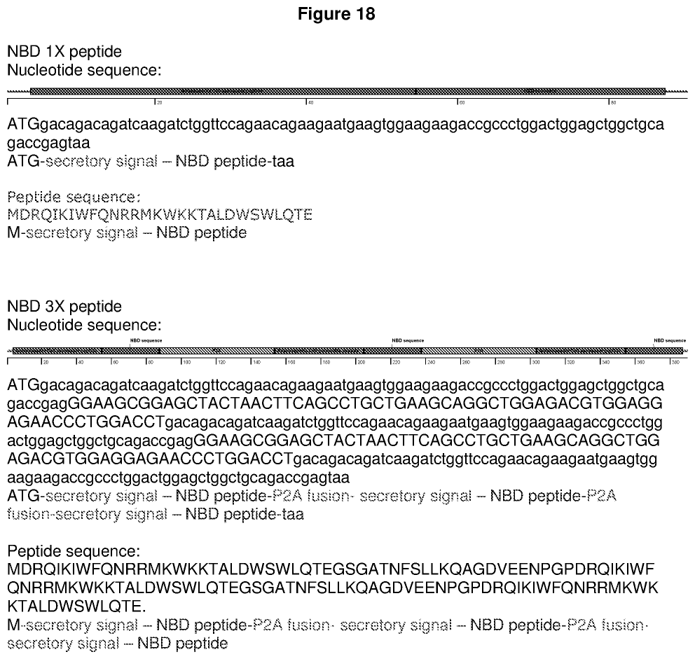

Another example of an anti-inflammatory protein or peptide provided by the disclosure is the Nemo binding domain (NBD) peptide. FIG. 18 sets out DNA and amino acid sequences of the NBD peptide (“NBD 1X peptide”) and an auto-cleaving fusion protein comprising three copies of the NBD peptide (“NBD 3X peptide”).

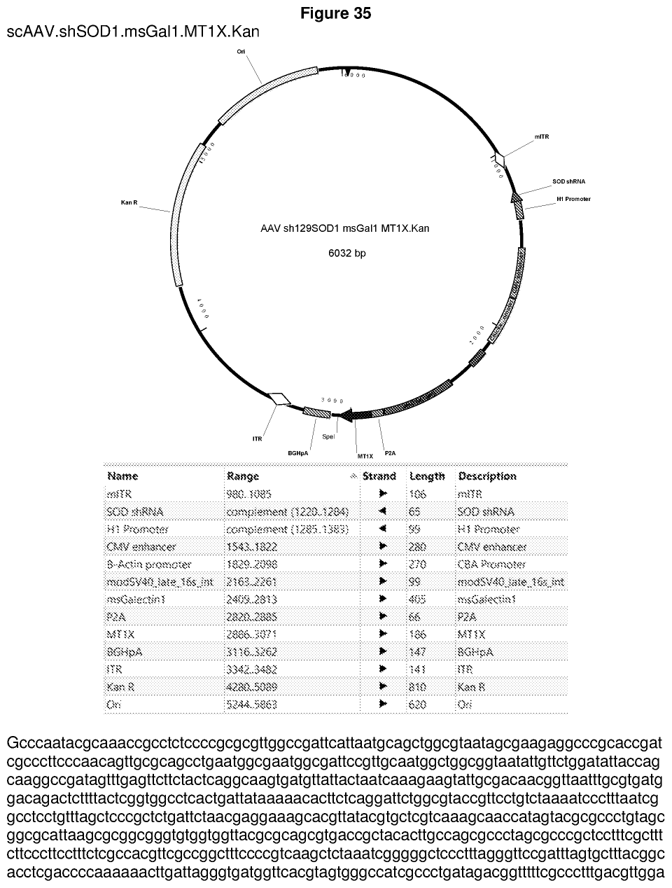

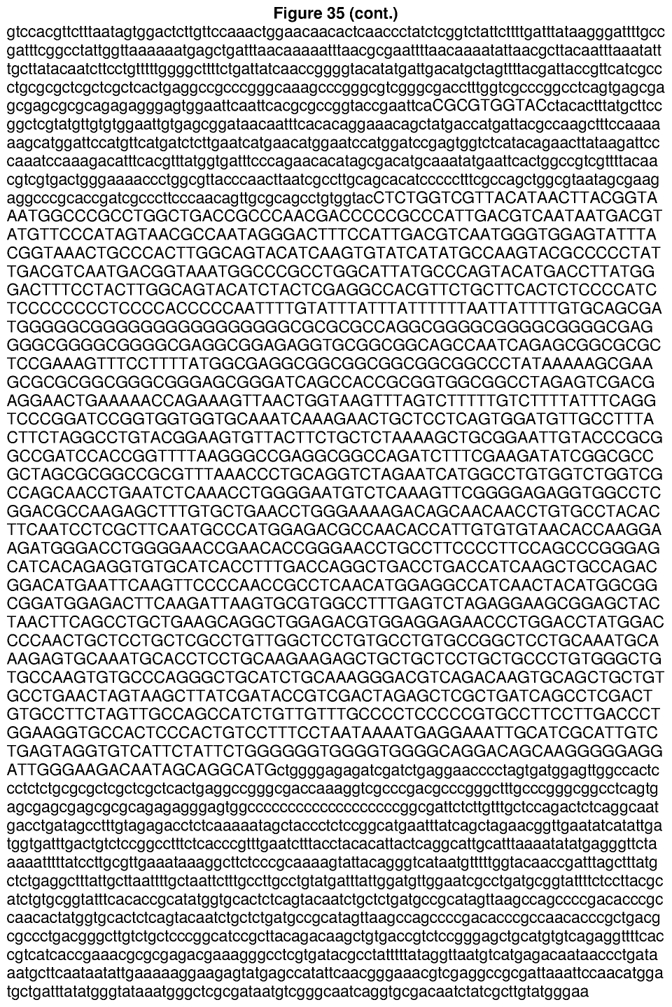

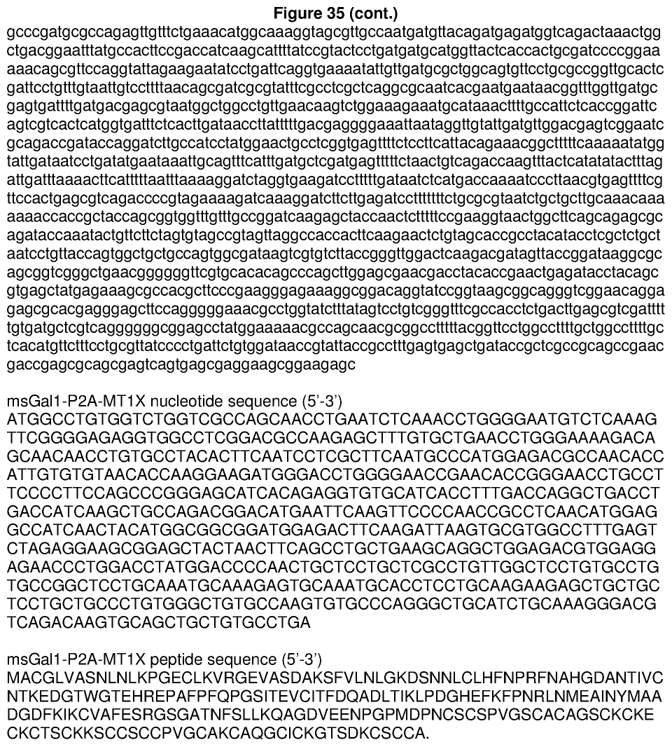

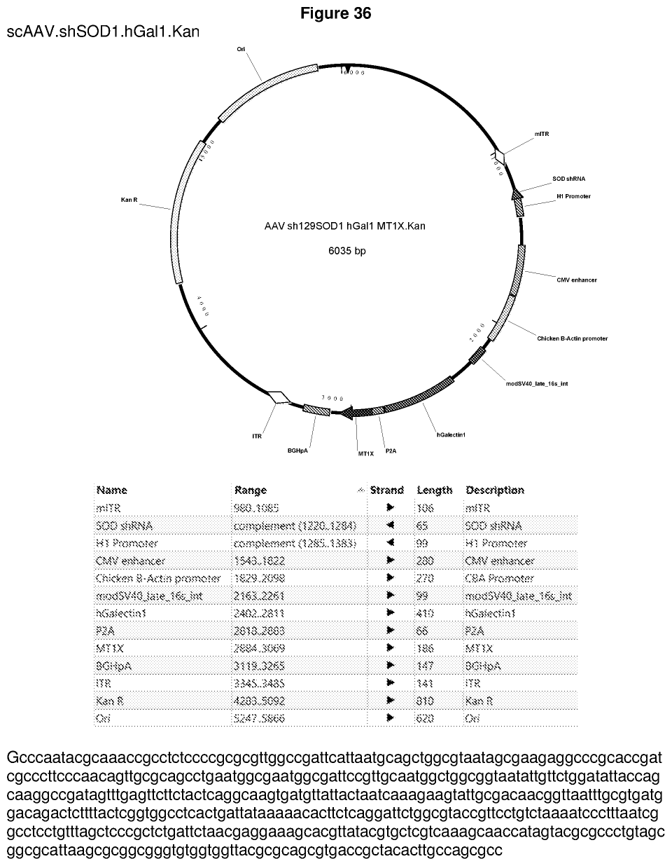

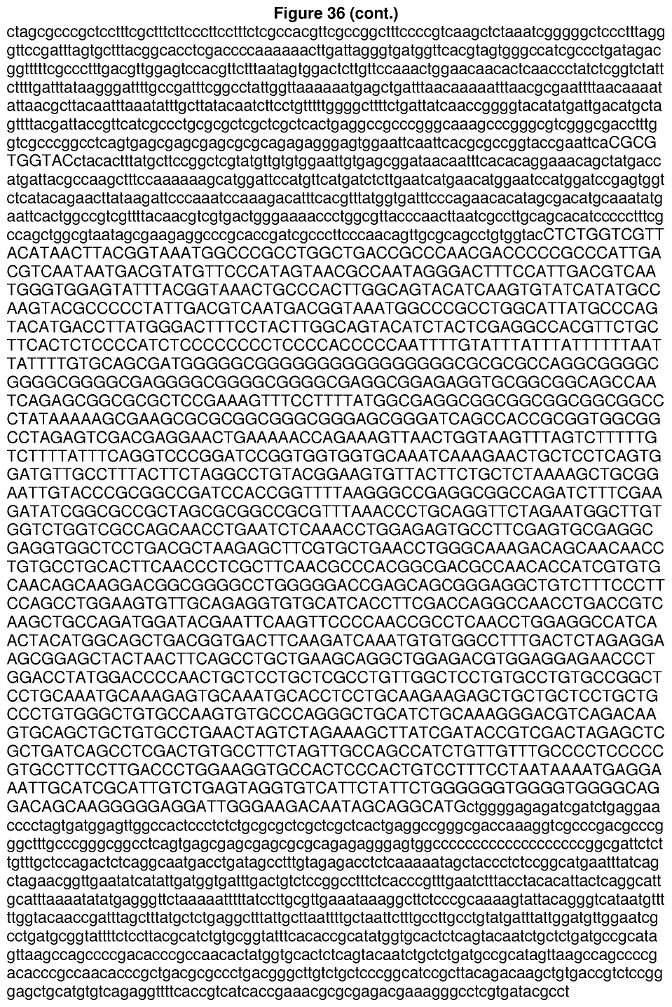

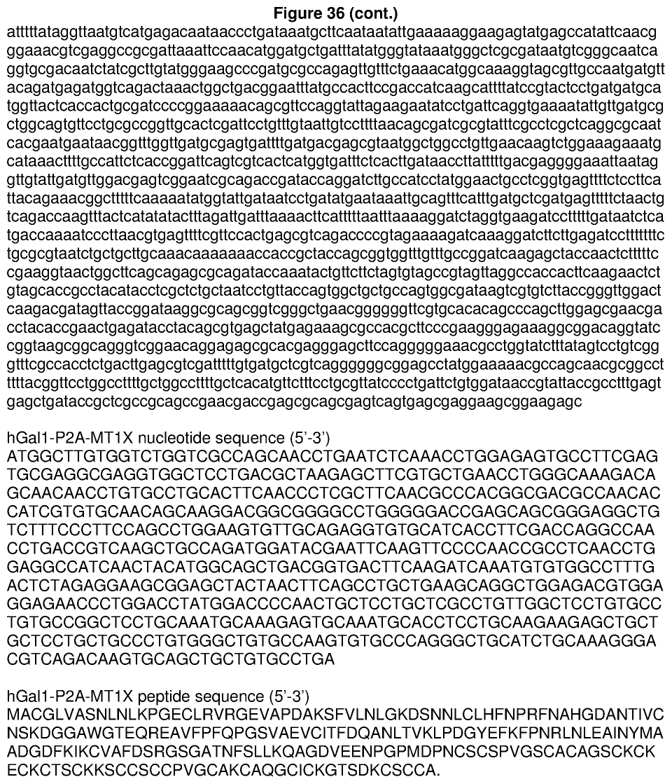

Yet another example of an anti-inflammatory protein or peptide provided by the disclosure is a metallothionein (MT) protein. FIGS. 35-37 show illustrative plasmids and sequences for making rAAV expressing a metallothionein (MT) protein.

AAV is a replication-deficient parvovirus, the single-stranded DNA genome of which is about 4.7 kb in length including 145 nucleotide inverted terminal repeat (ITRs).

There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45:555-564 (1983) as corrected by Ruffing et al., J Gen Virol, 75:3385-3392 (1994); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78:6381-6388 (2004); the AAV-10 genome is provided in Mol. Ther., 13 (1): 67-76 (2006); the AAV-11 genome is provided in Virology, 330 (2): 375-383 (2004); portions of the AAV-12 genome are provided in Genbank Accession No. DQ813647; portions of the AAV-13 genome are provided in Genbank Accession No. EU285562. The sequence of the AAV rh.74 genome is provided in U.S. Pat. No. 9,434,928. The sequence of the AAV-B1 genome is provided in Choudhury et al., Mol. Ther., 24 (7): 1247-1257 (2016). The sequence of Anc80 is provided in Zinn et al., Cell Reports 12:1056-1068, 2015 and Vandenberghe et al, PCT/US2014/060163, and GenBank Accession Nos. KT235804-KT235812. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158:97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as an expression cassette containing a promoter, a DNA of interest and a polyadenylation signal. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

The different AAV serotypes offer varied tissue tropism. Advances in the delivery of AAV6 and AAV8 have made possible the transduction by these serotypes of skeletal and cardiac muscle following simple systemic intravenous or intraperitoneal injections. See Pacak et al., Circ. Res., 99(4): 3-9 (1006) and Wang et al., Nature Biotech., 23(3): 321-8 (2005). The use of AAV to target cell types within the central nervous system has involved surgical intraparenchymal injection. See, Kaplitt et al., supra; Marks et al., supra and Worgall et al., supra. Regarding the use of AAV to target cell types within the nervous system, see International Publication No. WO 2010/071832. International Publication Nos. WO 2009/043936 and WO 2009/013290 state they relate to delivering genes to the central nervous system. International Publication No. WO 2011/133890 states it relates to recombinant adeno-associated viruses useful for targeting transgenes to central nervous system tissue.

The rAAV genomes of the disclosure lack AAV rep and cap DNA. AAV DNA in the rAAV genomes (e.g., ITRs) may be from any AAV serotype for which a recombinant virus can be derived including, but not limited to, AAV serotypes AAV1, AAV2, AAV3, AAV4, AAV5, AAV6, AAV7, AAV8, AAV9, AAV.rh74, AAV.rh8, AAV.rh10, AAV11, AAV12, AAV13, AAV-anc80, AAV-B1, AAV.PHP.EB, AAV7m8, or AAVv66. The rAAV genomes comprise at least one, or both, endogenous 5′ and 3′ inverted terminal repeats (ITRs). The rAAV genome can comprise ITRs from an AAV serotype that is different from the AAV serotype from which the AAV genome was derived. The rAAV genome can comprise three ITRs (e.g., as in scAAV).

The disclosure provides DNA plasmids comprising rAAV genomes of the disclosure. The DNA plasmids are transferred to cells permissible for infection with a helper virus of AAV (e.g., adenovirus, E1-deleted adenovirus or herpesvirus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep genes may be from any AAV serotype for which recombinant virus can be derived and may be from a different AAV serotype than the rAAV genome ITRs, including, but not limited to, AAV serotypes AAV1, AAV2, AAV3, AAV4, AAV5, AAV6, AAV7, AAV8, AAV9, AAV.rh74, AAV.rh8, AAV.rh10, AAV11, AAV12, AAV13, AAV-anc80, AAV-B1, AAV.PHP.EB, AAV7m8, or AAVv66. The ITRs in the AAV can be from a different AAV serotype. The AAV can comprise an ITR or capsid protein which is from a different serotype, i.e., a different serotype than the rest of the vector. For example, AAV2 or AAV2-based ITRs can be used in various AAV vector serotypes, not only serotypes which are AAV2 or AAV2-based. Thus, AAV2 ITRs can be used in a different serotype of AAV vector including, but not limited to, for example, AAV9. AAV2 Rep helper genes can be used. Production of pseudotyped rAAV is disclosed in, for example, WO 01/83692 which is incorporated by reference herein in its entirety.

AAV capsid proteins may be modified to enhance delivery of the recombinant vector. Modifications to capsid proteins are generally known in the art. See, for example, US 20050053922 and US 20090202490. See, for example, Marsic et al., Molecular Therapy 22(11): 1900-1909 (2014). Modified capsids provided herein can include capsids having various post-translational modifications such as glycosylation and deamidation. Deamidation of asparagine or glutamine side chains resulting in conversion of asparagine residues to aspartic acid or isoaspartic acid residues, and conversion of glutamine to glutamic acid or isoglutamic acid is contemplated in rAAV capsids provided herein. See, for example, Giles et al., Molecular Therapy, 26(12): 2848-2862 (2018). Modified capsids herein provided herein can also comprise targeting sequences directing the rAAV to the affected tissues and organs requiring treatment.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a kanamycin or neomycin resistance gene, are integrated into the genome of a cell. For example, the kanamycin resistance gene can replace the ampicillin resistance gene in the exemplary plasmids provided herein. AAV genomes have been introduced into plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy and Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

“Packaging” refers to a series of intracellular events that result in the assembly and encapsidation of an AAV particle. The term “production” refers to the process of producing the rAAV (the infectious, encapsulated rAAV particles) by the packing cells.

AAV “rep” and “cap” genes refer to polynucleotide sequences encoding replication and encapsidation proteins, respectively, of adeno-associated virus. AAV rep and cap are referred to herein as AAV “packaging genes.”

A “helper virus” for AAV refers to a virus that allows AAV (e.g. wild-type AAV) to be replicated and packaged by a mammalian cell. A variety of such helper viruses for AAV are known in the art, including adenoviruses, herpesviruses and poxviruses such as vaccinia. The adenoviruses may encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

“Helper virus function(s)” refers to function(s) encoded in a helper virus genome which allow AAV replication and packaging (in conjunction with other requirements for replication and packaging described herein). As described herein, “helper virus function” may be provided in a number of ways, including by providing helper virus or providing, for example, polynucleotide sequences encoding the requisite function(s) to a producer cell in trans.

General principles of rAAV production are reviewed in, for example, Carter, Current Opinions in Biotechnology, 1533-1539 (1992); and Muzyczka, Curr. Topics in Microbiol. and Immunol., 158:97-129 (1992). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); Mclaughlin et al., J. Virol., 62:1963 (1988); and Lebkowski et al., Mol. Cell. Biol., 7:349 (1988). Samulski et al., J. Virol., 63:3822-3828 (1989); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al., Vaccine, 13:1244-1250 (1995); Paul et al., Hum. Gene Ther., 4:609-615 (1993); Clark et al., Gene Ther., 3:1124-1132 (1996); U.S. Pat. Nos. 5,786,211; 5,871,982; 6,258,595; and McCarty, Mol. Ther., 16 (10): 1648-1656 (2008). Recombinant linear AAV (rAAV), single-stranded AAV (ssAAV), and self-complementary AAV (scAAV) are all specifically provided.

The disclosure thus provides packaging cells that produce infectious encapsidated rAAV particles. Packaging cells can be stably transformed cancer cells such as HeLa cells, 293 cells and PerC.6 cells (a cognate 293 line). Packaging cells can be cells that are not transformed cancer cells such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

The disclosure provides rAAV comprising a rAAV genome of the disclosure. The genomes of the rAAV lack AAV rep and cap DNA, that is, there is no AAV rep or cap DNA between the ITRs of the genomes. Between the ITRs, rAAV genomes of the disclosure comprise (1) an “expression cassette” comprising a DNA encoding a therapeutic protein or RNA that is operatively linked to transcriptional control elements that are functional in the target cells of interest (including, but not limited to, promoters, enhancers, and/or introns), and (2) an “expression cassette” comprising a galectin DNA (e.g., Galectin-1 DNA) that is operatively linked to transcriptional control elements that are functional in the target cells of interest (including, but not limited to, promoters, enhancers, and/or introns).

A “therapeutic protein or RNA” herein is a protein or RNA that corrects or ameliorates a genetic defect in a subject.

An “anti-inflammatory protein or peptide” herein is a protein or peptide herein that has anti-inflammatory activity such as maintaining microglia in a non-inflamed state or restoring microglia to a non-inflamed state.

In nature, the U6 promoter controls expression of the U6 RNA, a small nuclear RNA (snRNA) involved in splicing, and which has been well-characterized [Kunkel et al., Nature, 322(6074): 73-77 (1986); Kunkel et al., Genes Dev. 2 (2): 196-204 (1988); Paule et al., Nuc. Acids Res., 28(6): 1283-1298 (2000)]. The U6 promoter is used to control vector-based expression in mammalian cells [Paddison et al., Proc. Natl. Acad. Sci. USA, 99(3): 1443-1448 (2002); Paul et al., Nat. Biotechnol., 20(5): 505-518 (2002)] because (1) the promoter is recognized by RNA polymerase III (poly III) and controls high-level, constitutive expression of RNA; and (2) the promoter is active in most mammalian cell types. Use of murine or human U6 promoters to express therapeutic RNAs is contemplated by the disclosure.

U6 Promoter
(SEQ ID NO: 24)
5′CCCCAGTGGAAAGACGCGCAGGCAAAACGCACCACGTGACGGAGCGTG
ACCGCGCGCCGAGCGCGCGCCAAGGTCGGGCAGGAAGAGGGCCTATTTCC
CATGATTCCTTCATATTTGCATATACGATACAAGGCTGTTAGAGAGATAA
TTAGAATTAATTTGACTGTAAACACAAAGATATTAGTACAAAATACGTGA
CGTAGAAAGTAATAATTTCTTGGGTAGTTTGCAGTTTTAAAATTATGTTT
TAAAATGGACTATCATATGCTTACCGTAACTTGAAAGTATTTCGATTTCT
TGGGTTTATATATCTTGTGGAAAGGACGCGGGATC3′

As another example, an H1 promoter can be used to express an RNA.

H1 Promoter
(SEQ ID NO: 25)
5′AATTCATATTTGCATGTCGCTATGTGTTCTGGGAAATCACCATAAACG
TGAAATGTCTTTGGATTTGGGAATCTTATAAGTTCTGTATGAGACCACTC
G3′

Other promoters known in the art are provided herein for expressing therapeutic proteins or anti-inflammatory proteins/peptides. Examples of such promoters provided are: the chicken β actin promoter (CBA),

CAG Promoter (CMV enhancer + CBA promoter)
(SEQ ID NO: 26)
5′CGTTACATAACTTACGGTAAATGGCCCGCCTGGCTGACCGCCCAACGA
CCCCCGCCCATTGACGTCAATAATGACGTATGTTCCCATAGTAACGCCAA
TAGGGACTTTCCATTGACGTCAATGGGTGGAGTATTTACGGTAAACTGCC
CACTTGGCAGTACATCAAGTGTATCATATGCCAAGTACGCCCCCTATTGA
CGTCAATGACGGTAAATGGCCCGCCTGGCATTATGCCCAGTACATGACCT
TATGGGACTTTCCTACTTGGCAGTACATCTACTCGAGGCCACGTTCTGCT
TCACTCTCCCCATCTCCCCCCCCTCCCCACCCCCAATTTTGTATTTATTT
ATTTTTTAATTATTTTGTGCAGCGATGGGGGGGGGGGGGGGGGGGGGGGG
CGCGCCAGGCGGGGGGGGGGGGGGCGAGGGGGGGGGGGGGCGAGGCGGAG
AGGTGCGGCGGCAGCCAATCAGAGCGGCGCGCTCCGAAAGTTTCCTTTTA
TGGCGAGGCGGCGGCGGCGGCGGCCCTATAAAAAGCGAAGCGCGCGGCGG
GCGGGAG3′,

a truncated methyl CpG binding protein 2 (MeCP2) promoter called the P546 MeCP2 promoter (for driving expression in, for example, neurons and astrocytes),

P546 Promoter
(SEQ ID NO: 27)
5′TTTTCCGGACGGGTTTTACCACAGCCCTCTCTCCGAGAGGAGGGAGCG
CGCGCGCAACCGATGCCGGGACCCCGCACGGCAGACGTCGCGCCCCGCCC
TCCCGACCAGCCTGTGTGCTGCTGCACCTGCGCGCCCGCGCCCCACCCCT
TGCTCTTTGTCGAGATTACCCTTCATTGGTTGTGGAGCCCAGGCTGGGGC
GGAGCCTTAGCGGTGACGCCCTCAATTGGCAGGAGTTCCTGTCTGTTTAG
GCAGGGAAAAGAGGCGGACCCCATTCAGCTGCGGATTGGTGGAGTTCTAC
TGTCACTTGGAAAAAAGAGGCGGCTAGGGCACAGAGGGGCTGGTTTTGTG
GGCAGCATTTGAATGTTGAGGATTAACTGGGCCCTTGTGGACTCTGGCGC
TTAAGGAAGTCTAGGCTCTTGGCGCCTATTAGAGCCTCCCTGCTGAGTAG
TTCACCATTGTGATAAGCATTTGACTTCACCAGCATTTCTTTATTATCAT
TTTCTGTAGAAGTAGCAAAGTTGCCTGTTGAGGAGCCTGGCGTTGTTC
3′,

a human synapsin (hSyn) promoter (for driving expression, for example, in neurons)

hSyn promoter
(SEQ ID NO: 28)
5′GTGTCTAGAC TGCAGAGGGC CCTGCGTATG AGTGCAAGTG
GGTTTTAGGA CCAGGATGAG GCGGGGTGGG GGTGCCTACC
TGACGACCGA CCCCGACCCA CTGGACAAGC ACCCAACCCC
CATTCCCCAA ATTGCGCATC CCCTATCAGA GAGGGGGAGG
GGAAACAGGA TGCGGCGAGG CGCGTGCGCA CTGCCAGCTT
CAGCACCGCG GACAGTGCCT TCGCCCCCGC CTGGCGGCGC
GCGCCACCGC CGCCTCAGCA CTGAAGGCGC GCTGACGTCA
CTCGCCGGTC CCCCGCAAAC TCCCCTTCCC GGCCACCTTG
GTCGCGTCCG CGCCGCCGCC GGCCCAGCCG GACCGCACCA
CGCGAGGCGC GAGATAGGGG GGCACGGGCG CGACCATCTG
CGCTGCGGCG CCGGCGACTC AGCGCTGCCT CAGTCTGCGG
TGGGCAGCGG AGGAGTCGTG TCGTGCCTGA GAGCGCAGTC
GAGAA3′,

the human somatostatin (hSST) promoter (for driving expression, for example, in inhibitory neurons),

hSST promoter
(SEQ ID NO: 29)
5′GCATGTGTGG GAGTGAAATT ATGGAATGTG TATGCTCATA
GCACTGAGTG AAAATAAAAG ATTGTATAAA TCGTGGGGCT
TGTGGAATTG TGAGTCCCTG TGCGTGTGCA GTATTTTTTT
TTTTTTTTAA GTAAGACTCT TTAGATCTTG TCGCCTCCCC
TGTCTTCTGT GATTGATTTT GCGAGACTAA TGGTGCGTAA
AAGGGCTGGT GAGATCTGGG GGCGCCTCCT AGCCTGACGT
CAGAGAGAGA GTTTAAAACC GAGGGAGACG GTTGAGAGCA
CACAAGCCGC TTTAGGAGTC GCGAGGTTCG GAGCCATCGC
TGCTGCCTGC TGATCCGCGC CTAGAGTTTG3′,

the compact glial fibrillary acidic protein [gfaABC(1)D] promoter (for driving expression, for example, in astrocytes),

gfaABC(1)D promoter
(SEQ ID NO: 30)
5′AACATATCCTGGTGTGGAGTAGGGGACGCTGCTCTGACAGAGGCTCGG
GGGCCTGAGCTGGCTCTGTGAGCTGGGGAGGAGGCAGACAGCCAGGCCTT
GTCTGCAAGCAGACCTGGCAGCATTGGGCTGGCCGCCCCCCAGGGCCTCC
TCTTCATGCCCAGTGAATGACTCACCTTGGCACAGACACAATGTTCGGGG
TGGGCACAGTGCCTGCTTCCCGCCGCACCCCAGCCCCCCTCAAATGCCTT
CCGAGAAGCCCATTGAGCAGGGGGCTTGCATTGCACCCCAGCCTGACAGC
CTGGCATCTTGGGATAAAAGCAGCACAGCCCCCTAGGGGCTGCCCTTGCT
GTGTGGCGCCACCGGCGGTGGAGAACAAGGCTCTATTCAGCCTGTGCCCA
GGAAAGGGGATCAGGGGATGCCCAGGCATGGACAGTGGGTGGCAGGGGGG
GAGAGGAGGGCTGTCTGCTTCCCAGAAGTCCAAGGACACAAATGGGTGAG
GGGAGAGCTCTCCCCATAGCTGGGCTGCGGCCCAACCCCACCCCCTCAGG
CTATGCCAGGGGGTGTTGCCAGGGGCACCCGGGCATCGCCAGTCTAGCCC
ACTCCTTCATAAAGCCCTCGCATCCCAGGAGCGAGCAGAGCCA3′,

the glial fibrillary acidic protein (GFAP) promoter,

GFAP promoter
(SEQ ID NO: 31)
5′ACGCGTCCCACCTCCCTCTCTGTGCTGGGACTCACAGAGGGAGACCTC
AGGAGGCAGTCTGTCCATCACATGTCCAAATGCAGAGCATACCCTGGGCT
GGGCGCAGTGGCGCACAACTGTAATTCCAGCACTTTGGGAGGCTGATGTG
GAAGGATCACTTGAGCCCAGAAGTTCTAGACCAGCCTGGGCAACATGGCA
AGACCCTATCTCTACAAAAAAAGTTAAAAAATCAGCCACGTGTGGTGACA
CACACCTGTAGTCCCAGCTATTCAGGAGGCTGAGGTGAGGGGATCACTTA
AGGCTGGGAGGTTGAGGCTGCAGTGAGTCGTGGTTGCGCCACTGCACTCC
AGCCTGGGCAACAGTGAGACCCTGTCTCAAAAGACAAAAAAAAAAAAAAA
AAAAAAAAGAACATATCCTGGTGTGGAGTAGGGGACGCTGCTCTGACAGA
GGCTCGGGGGCCTGAGCTGGCTCTGTGAGCTGGGGAGGAGGCAGACAGCC
AGGCCTTGTCTGCAAGCAGACCTGGCAGCATTGGGCTGGCCGCCCCCCAG
GGCCTCCTCTTCATGCCCAGTGAATGACTCACCTTGGCACAGACACAATG
TTCGGGGTGGGCACAGTGCCTGCTTCCCGCCGCACCCCAGCCCCCCTCAA
ATGCCTTCCGAGAAGCCCATTGAGCAGGGGGCTTGCATTGCACCCCAGCC
TGACAGCCTGGCATCTTGGGATAAAAGCAGCACAGCCCCCTAGGGGCTGC
CCTTGCTGTGTGGCGCCACCGGCGGTGGAGAACAAGGCTCTATTCAGCCT
GTGCCCAGGAAAGGGGATCAGGGGATGCCCAGGCATGGACAGTGGGTGGC
AGGGGGGGAGAGGAGGGCTGTCTGCTTCCCAGAAGTCCAAGGACACAAAT
GGGTGAGGGGACTGGGCAGGGTTCTGACCCTGTGGGACCAGAGTGGAGGG
CGTAGATGGACCTGAAGTCTCCAGGGACAACAGGGCCCAGGTCTCAGGCT
CCTAGTTGGGCCCAGTGGCTCCAGCGTTTCCAAACCCATCCATCCCCAGA
GGTTCTTCCCATCTCTCCAGGCTGATGTGTGGGAACTCGAGGAAATAAAT
CTCCAGTGGGAGACGGAGGGGTGGCCAGGGAAACGGGGCGCTGCAGGAAT
AAAGACGAGCCAGCACAGCCAGCTCATGTGTAACGGCTTTGTGGAGCTGT
CAAGGCCTGGTCTCTGGGAGAGAGGCACAGGGAGGCCAGACAAGGAAGGG
GTGACCTGGAGGGACAGATCCAGGGGCTAAAGTCCTGATAAGGCAAGAGA
GTGCCGGCCCCCTCTTGCCCTATCAGGACCTCCACTGCCACATAGAGGCC
ATGATTGACCCTTAGACAAAGGGCTGGTGTCCAATCCCAGCCCCCAGCCC
CAGAACTCCAGGGAATGAATGGGCAGAGAGCAGGAATGTGGGACATCTGT
GTTCAAGGGAAGGACTCCAGGAGTCTGCTGGGAATGAGGCCTAGTAGGAA
ATGAGGTGGCCCTTGAGGGTACAGAACAGGTTCATTCTTCGCCAAATTCC
CAGCACCTTGCAGGCACTTACAGCTGAGTGAGATAATGCCTGGGTTATGA
AATCAAAAAGTTGGAAAGCAGGTCAGAGGTCATCTGGTACAGCCCTTCCT
TCCCTTTTTTTTTTTTTTTTTGTGAGACAAGGTCTCTCTCTGTTGCCCAG
GCTGGAGTGGCGCAAACACAGCTCACTGCAGCCTCAACCTACTGGGCTCA
AGCAATCCTCCAGCCTCAGCCTCCCAAAGTGCTGGGATTACAAGCATGAG
CCACCCCACTCAGCCCTTTCCTTCCTTTTTAATTGATGCATAATAATTGT
AAGTATTCATCATGGTCCAACCAACCCTTTCTTGACCCACCTTCCTAGAG
AGAGGGTCCTCTTGCTTCAGCGGTCAGGGCCCCAGACCCATGGTCTGGCT
CCAGGTACCACCTGCCTCATGCAGGAGTTGGCGTGCCCAGGAAGCTCTGC
CTCTGGGCACAGTGACCTCAGTGGGGTGAGGGGAGCTCTCCCCATAGCTG
GGCTGCGGCCCAACCCCACCCCCTCAGGCTATGCCAGGGGGTGTTGCCAG
GGGCACCCGGGCATCGCCAGTCTAGCCCACTCCTTCATAAAGCCCTCGCA
TCCCAGGAGCGAGCAGAGCCAGAGCAGGTTGGAGAGGAGACGCATCACCT
CCGCTGCTCGCCGG3′,
the tMCK promoter,
(SEQ ID NO: 32)
5′CCACTACGGGTCTAGGCTGCCCATGTAAGGAGGCAAGGCCTGGGGACA
CCCGAGATGCCTGGTTATAATTAACCCCAACACCTGCTGCCCCCCCCCCC
CCAACACCTGCTGCCTGAGCCTGAGCGGTTACCCCACCCCGGTGCCTGGG
TCTTAGGCTCTGTACACCATGGAGGAGAAGCTCGCTCTAAAAATAACCCT
GTCCCTGGTGGANCCACTACGGGTCTANGCTGCCCATGTAAGGAGGCAAG
GCCTGGGGACACCCGAGATGCCTGGTTATAATTAACCCCAACACCTGCTG
CCCCCCCCCCCCCAACACCTGCTGCCTGAGCCTGAGCGGTTACCCCACCC
CGGTGCCTGGGTCTTAGGCTCTGTACACCATGGAGGAGAAGCTCGCTCTA
AAAATAACCCTGTCCCTGGTGGACCACTACGGGTCTAGGCTGCCCATGTA
AGGAGGCAAGCCTGGGGACACCCGAGATGCCTGGTTATAATTAACCCCAA
CACCTGCTGCCCCCCCCCCCCAACACCTGCTGCCTGAGCCTGAGCGGTTA
CCCCACCCCGGTGCCTGGGTCTTAGGCTCTGTACACCATGGAGGAGAAGC
TCGCTCTAAAAATAACCCTGTCCCTGGTCCTCCCTGGGGACAGCCCCTCC
TGGCTAGTCACACCCTGTAGGCTCCTCTATATAACCCAGGGGCACAGGGG
CTGCCCCCGGGTCACT3′
the MHCk promoter,
(SEQ ID NO: 33)
5′CATGTCTAAGCTAGACCCTTCAGATTAAAAATAACTGAGGTAAGGGCC
TGGGTAGGGGAGGTGGTGTGAGACGCTCCTGTCTCTCCTCTATCTGCCCA
TCGGCCCTTTGGGGAGGAGGAATGTGCCCAAGGACTAAAAAAAGGCCATG
GAGCCAGAGGGGCGAGGGCAACAGACCTTTCATGGGCAAACCTTGGGGCC
CTGCTGTCTAGCATGCCCCACTACGGGTCTAGGCTGCCCATGTAAGGAGG
CAAGGCCTGGGGACACCCGAGATGCCTGGTTATAATTAACCCAGACATGT
GGCTGCCCCCCCCCCCCCAACACCTGCTGCCTCTAAAAATAACCCTGTCC
CTGGTGGATCCCCTGCATGCGAAGATCTTCGAACAAGGCTGTGGGGGACT
GAGGGCAGGCTGTAACAGGCTTGGGGGCCAGGGCTTATACGTGCCTGGGA
CTCCCAAAGTATTACTGTTCCATGTTCCCGGCGAAGGGCCAGCTGTCCCC
CGCCAGCTAGACTCAGCACTTAGTTTAGGAACCAGTGAGCAAGTCAGCCC
TTGGGGCAGCCCATACAAGGCCATGGGGCTGGGCAAGCTGCACGCCTGGG
TCCGGGGTGGGCACGGTGCCCGGGCAACGAGCTGAAAGCTCATCTGCTCT
CAGGGGCCCCTCCCTGGGGACAGCCCCTCCTGGCTAGTCACACCCTGTAG
GCTCCTCTATATAACCCAGGGGCACAGGGGCTGCCCTCATTCTACCACCA
CCTCCACAGCACAGACA GACACTCAGGAGCAGC3′

the IRF promoter, the neuron-specific enolase promoter, the CMV promoter, and the Myo7A promoter. Additional promoters are contemplated herein including, but not limited to, the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as the actin promoter, the myosin promoter, the elongation factor-1a promoter, the hemoglobin promoter, and the creatine kinase promoter.

Inducible promoters are also provided. Non-limiting examples of inducible promoters include, but are not limited to a metallothionine promoter, a glucocorticoid promoter, a progesterone promoter, and a tetracycline-regulated promoter.

Expression cassettes may also include intron sequences to facilitate processing of a transgene RNA transcript when expressed in mammalian cells. One example of such an intron is the SV40 intron.

Recombinant AAV (rAAV) (i.e., infectious encapsidated rAAV particles) (sometimes referred to herein as “vectors”) are thus provided herein. The genomes of the rAAV lack AAV rep and cap DNA, that is, there is no AAV rep or cap DNA between the ITRs of the genomes of the rAAV.

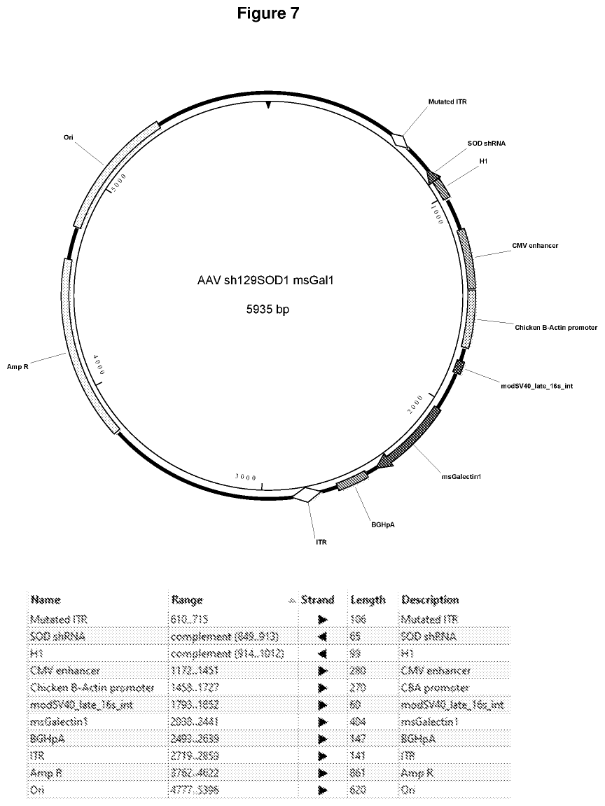

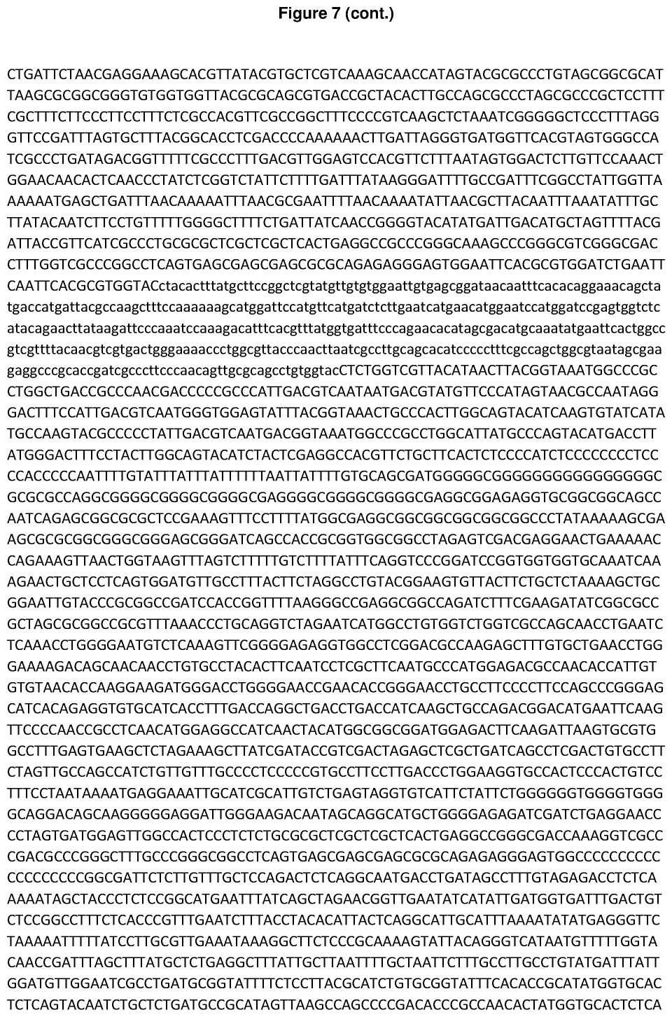

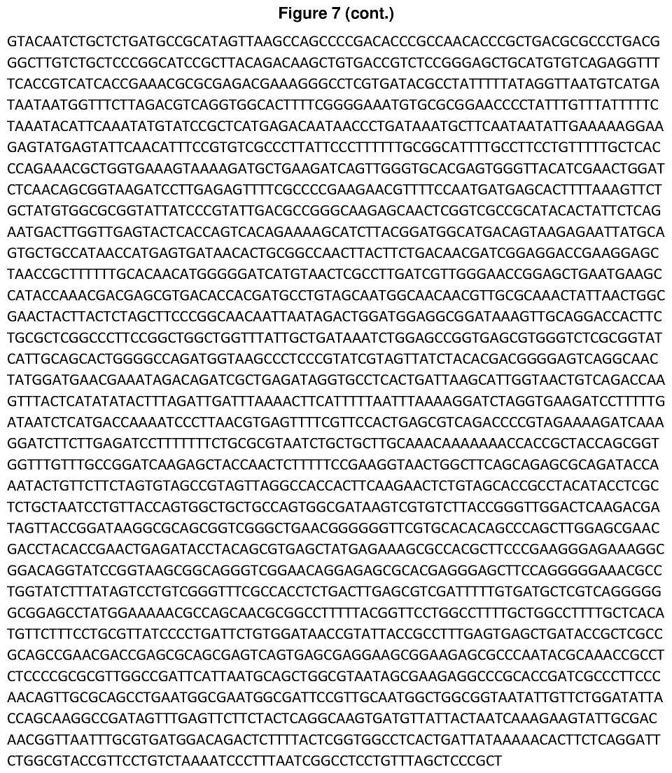

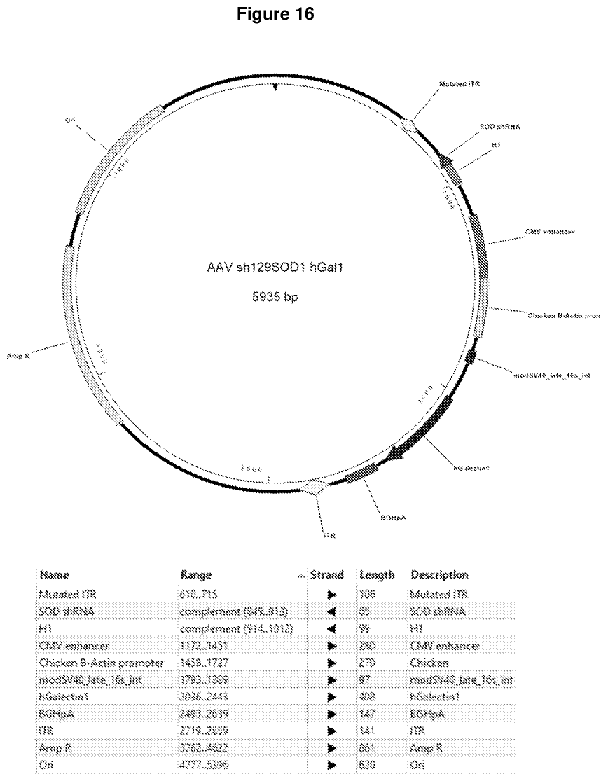

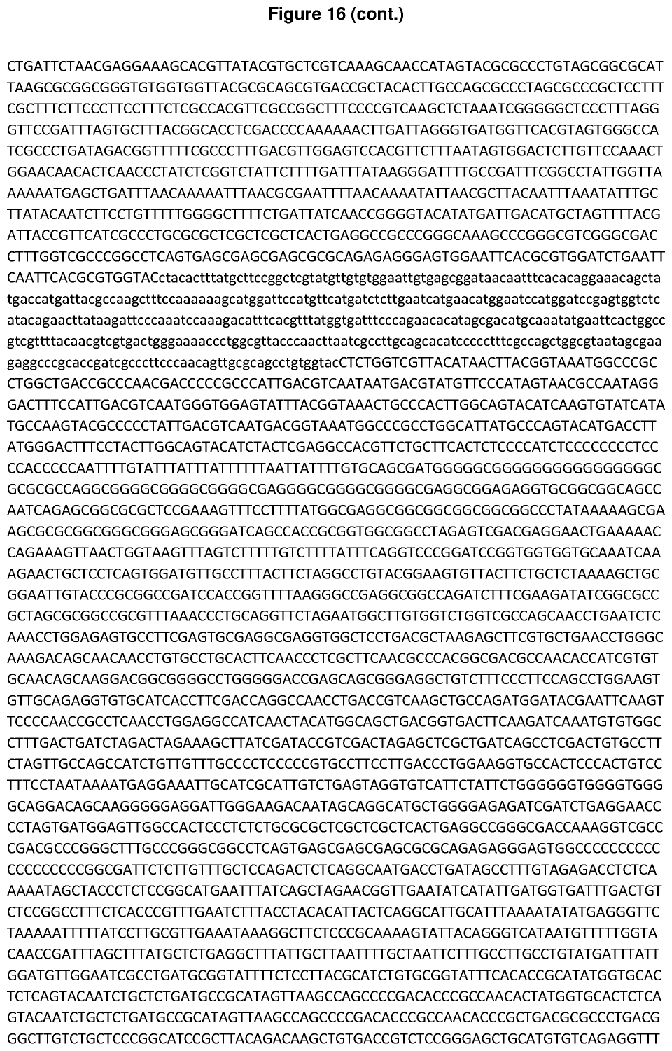

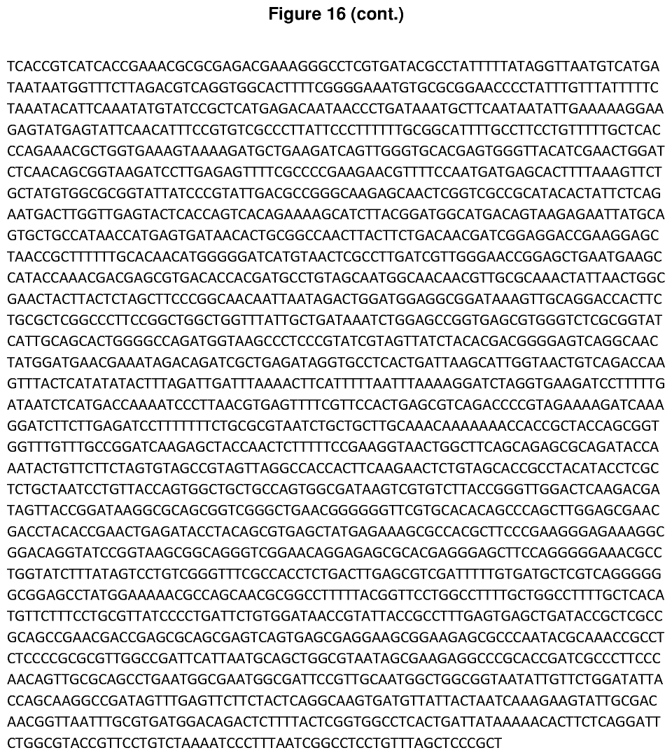

rAAV provided for treatment of ALS include, but are not limited to, an exemplary rAAV including a genome comprising an expression cassette encoding a SOD1 shRNA 129 and an expression cassette encoding human Galectin-1 named “AAV.sh129SOD1.hGal1”, and an exemplary rAAV including a genome comprising an expression cassette encoding the SOD1 shRNA 129 and an expression cassette encoding mouse Galectin-1 named “AAV.sh129SOD1.msGal1”. Plasmids used to produce the two exemplary rAAV are respectively shown in FIGS. 16 and 7. The SOD shRNA 129 nucleotides in the two AAV genomes comprise the entire hairpin sequence including the sense and antisense arms, stem loop and termination sequence. The sequence in a forward orientation (with target sequences against SOD1 underlined) is:

(SEQ ID NO: 34)
5′AATTCATATTTGCATGTCGCTATGTGTTCTGGGAAATCACCATAAACG
TGAAATGTCTTTGGATTTGGGAATCTTATAAGTTCTGTATGAGACCACTC
GGATCCATGGATTCCATGTTCATGATTCAAGAGATCATGAACATGGAATC
CATGCTTTTTTGGAAA 3′

The AAV.sh129SOD1.msGal1 transduces neurons, astrocytes and oligodendrocytes.

The disclosure contemplates that rAAV known in the art for treatment of DMD [rAAV encoding a micro-dystrophin, Mendell et al., JAMA Neurol., 77(9) 1122-1131 (2020)] [rAAV encoding a DMD exon 2 targeting U7snRNA described in WO2014/172669; Simmons et al., Mol. Ther.: Methods & Clinical Development, 21: 325-340 (2021); Gushchina et al., Human Gene Therapy, 32(17-18): 882-894 (2021)] [Wein et al., Nat. Med., 20(9): 992-1000 (2014)] and for the treatment of spinal muscular atrophy (SMA) [e.g., Zolgensma (onasemnogene abeparvovec-xioi), a rAAV encoding the human survival motor neuron (SMN) protein described in, e.g., Mendell et al., N. Engl. J. Med., 377:1713-1722 (2017)] can be modified to additionally include an anti-inflammatory protein/peptide (e.g., galectin) gene cassette as described herein. These rAAV for DMD transduce skeletal muscles and heart. The rAAV for SMA transduce neurons, astrocytes and oligodendrocytes.

The disclosure provides additional illustrative rAAV including, but not limited to, AAV.sh129SOD1.hGal1, AAV.hGalactin1, scAAV.P546.CLN1.Gal1, scAAV.CB. CLN1.Gal1, scAAV.P546.CLN3.Gal1, scAAV.CB.CLN3.Gal1, scAAV.CB.CLN6.Gal1, SCAAV.P546.CLN8.Gal1, scAAV.CB.CLN8.Gal1, scAAV.P546.IGHMBP2.Gal1, SCAAV.CB.IGHMBP2.Gal1, scAAV.546.PGAP3.Gal1 and scAAV.CBA.PGAP3.Gal1.

The rAAV of the disclosure may be purified by methods standard in the art such as by column chromatography or cesium chloride gradients. Methods for purifying rAAV vectors from helper virus are known in the art and include methods disclosed in, for example, Clark et al., Hum. Gene Ther., 10 (6): 1031-1039 (1999); Schenpp and Clark, Methods Mol. Med., 69:427-443 (2002); U.S. Pat. No. 6,566, 118 and WO 98/09657.

The disclosure provides compositions comprising rAAV of the present disclosure. Compositions of the disclosure comprise rAAV in a pharmaceutically acceptable carrier. The compositions may also comprise other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

Titers of rAAV to be administered in methods of the disclosure will vary depending, for example, on the particular rAAV, the mode of administration, the treatment goal, the individual, and the cell type(s) being targeted, and may be determined by methods standard in the art. Titers of rAAV may range from about 1×10², about 1×10³, about 1×10⁴, about 1×10⁵, about 1×10⁶, about 1×10⁷, about 1×10⁸, about 1×10⁹, about 1×10¹⁰, about 1×10¹¹, about 1×10¹², about 1×10¹³, to about 1×10¹⁴ or more DNase resistant particles (DRP) per ml. Dosages may also be expressed in units of viral genomes (vg). Dosages may also vary based on the timing of the administration to a human. These dosages of rAAV may range from about 1×10⁴, about 1×10⁵, about 1×10⁶, about 1×10⁷, about 1×10⁸, about 1×10⁹, about 1×10¹⁰, about 1×10¹¹, about 1×10¹², about 1×10¹³, about 1×10¹⁴, about 1×10¹⁵, to about 1×10¹⁶ or more viral genomes per kilogram body weight in an adult. For a neonate, the dosages of rAAV may range from about about 1×10⁴, about 3×10⁴, about 1×10⁵, about 3×10⁵, about 1×10⁶, about 3×10⁶, about 1×10⁷, about 3×10⁷, about 1×10⁸, about 3×10⁸, about 1×10⁹, about 3×10⁹, about 1×10¹⁰, about 3×10¹⁰, about 1×10¹¹, about 3×10¹¹, about 1×10¹², about 3×10¹², about 1×10¹³, about 3×10¹³, about 1×10¹⁴, about 3×10¹⁴, about 1×10¹⁵, about 3×10¹⁵ about 1×10¹⁶, to about 3×10¹⁶ or more viral genomes per kilogram body weight.

The disclosure provides methods of transducing a target cell with a rAAV of the disclosure, in vivo or in vitro. The in vivo methods comprise the step of administering an effective dose, or effective multiple doses, of a composition comprising a rAAV of the disclosure to a subject (including a human being), in need thereof. If the dose is administered prior to onset/development of a disorder/disease, the administration is prophylactic. If the dose is administered after the onset/development of a disorder/disease, the administration is therapeutic. An effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. An example of a disease contemplated for treatment with methods of the disclosure is ALS. Other examples are DMD and SMA. “Treatment” according to the disclosure thus alleviates (eliminates or reduces) at least one symptom associated with the disorder/disease state being treated (for example, weight loss is eliminated or reduced by at least 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or greater), that slows or prevents progression to (onset/development) of a disorder/disease state, that slows or prevents progression of a disorder/disease state, that diminishes the extent of disease, that results in remission (partial or total) of disease, and/or that prolongs survival. Survival can be prolonged by at least 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or greater.

Further combination therapies are also provided by the disclosure. Combination as used herein includes both simultaneous treatment or sequential treatments. For example, combinations of methods of the disclosure with standard medical treatments (e.g., riluzole and CuATSM (diacetylbis(N(4)-methylthiosemicarbazonato)copper (II)) for ALS] are specifically provided, as are combinations with novel therapies.

Administration of an effective dose of the compositions may be by routes standard in the art including, but not limited to, systemic intramuscular, parenteral, intravenous, oral, buccal, nasal, pulmonary, intracranial, intrathecal, intraosseous, intraocular, rectal, or vaginal. Route(s) of administration and serotype(s) of AAV components of the rAAV (in particular, the AAV ITRs and capsid protein) of the disclosure may be chosen and/or matched by those skilled in the art taking into account the infection and/or disease state being treated and the target cells/tissue(s) to be transduced. The route of administration can be systemic. The route of administration can be intrathecal. The route of administration can be intracerebroventricular. The route of administration can be cisterna magna. The route of administration can be by lumbar puncture.

For cerebrospinal fluid (CSF) delivery, including but not limited to intrathecal delivery, compositions provided herein can comprise a pharmaceutically acceptable aqueous excipient containing a non-ionic, low-osmolar compound or contrast agent such as iobitridol, iohexol, iomeprol, iopamidol, iopentol, iopromide, ioversol, or ioxilan, where the aqueous excipient containing the non-ionic, low-osmolar compound can have one or more of the following characteristics: about 180 mgl/mL, an osmolality by vapor-pressure osmometry of about 322 mOsm/kg water, an osmolarity of about 273 mOsm/L, an absolute viscosity of about 2.3 cp at 20° C. and about 1.5 cp at 37° C., and a specific gravity of about 1.164 at 37° C. Exemplary compositions comprise about 20 to 40% non-ionic, low-osmolar compound or about 25% to about 35% non-ionic, low-osmolar compound. An exemplary composition comprises scAAV or rAAV viral particles formulated in 20 mM Tris (pH8.0), 1 mM MgCl₂, 200 mM NaCl, 0.001% poloxamer 188 and about 25% to about 35% non-ionic, low-osmolar compound. Another exemplary composition comprises scAAV formulated in 1X PBS and 0.001% or 0.005% Pluronic F68.

For intrathecal administration, the subject can be held in the Trendelenburg position (head down position) after injection of the rAAV (e.g., for about 5, about 10, about 15 or about 20 minutes). For example, the patient may be tilted in the head down position at about 1 degree to about 30 degrees, about 15 to about 30 degrees, about 30 to about 60 degrees, about 60 to about 90 degrees, or about 90 to about 180 degrees.

Transduction of cells with rAAV of the disclosure results in sustained expression of the therapeutic protein/RNA and anti-inflammatory protein/peptide (e.g., SOD1 shRNA and galectin, respectively). The present disclosure thus provides methods of administering/delivering rAAV provided herein (e.g., which express SOD1 shRNA and galectin) to a subject, preferably a human being. The term “transduction” is used to refer to the administration/delivery of DNA encoding the therapeutic protein/RNA and anti-inflammatory protein/peptide to a recipient cell either in vivo or in vitro, via a replication-deficient rAAV of the disclosure resulting in expression of the therapeutic protein/RNA and anti-inflammatory protein/peptide by the recipient cell.

Thus, the disclosure provides methods of administering an effective dose (or doses, administered essentially simultaneously or doses given at intervals) of rAAV of the disclosure (e.g., that encode SOD1 shRNA and galectin) to a subject in need thereof.

Methods of the disclosure can be used to deliver polynucleotides to nerve, glial cells and endothelial cells. The nerve cell can be a lower motor neuron and/or an upper motor neuron. The glial cell can be a microglial cell, an oligodendrocyte and/or an astrocyte. The rAAV can be used to deliver a polynucleotide to a Schwann cell. The rAAV can be used to deliver a polynucleotide to a muscle or liver cell.

Methods of the disclosure can be used to treat a neurological or neurodegenerative disorder such as ALS, DMD, SMA, Batten disease (CLN1/3/6/8), IGHMBP2-related disorder (SMARD1/CMT2S), Pitt-Hopkins Syndrome and PGAP3 Congenital Disorder of Glycosylation in a subject. The methods comprise administering to the subject an effective amount of an rAAV composition provided herein.

### Other Terminology and Disclosure

As used herein and in the appended claims, the singular forms “a,” “and,” and “the” include plural referents unless the context clearly dictates otherwise. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

Throughout this specification and the claims which follow, unless the context requires otherwise, the word “comprise”, and variations such as “comprises” and “comprising”, will be understood to mean the inclusion of a stated component or step, or group of components or steps, but not the exclusion of any other component or step or group of components or steps.

When used herein, “consisting of” excludes any element, step, or ingredient not specified in the claim element. When used herein, “consisting essentially of” does not exclude materials or steps that do not materially affect the basic and novel characteristics of the claim element.

When a range of values is provided herein, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

The disclosure herein specifically contemplates fragments and variants of the genome components (e.g., the galectin DNA component or a promoter component) of the rAAV provided. For example, the disclosure contemplates a rAAV encoding a fragment or variant of Galectin-1 protein with at least 90%, 91%, 92%, 93%, 94%, 95%, 96%, 97%, 98%, 99% identity to the human Galectin-1 protein that retains Galectin-1 activity on microglia, and contemplates a rAAV comprising a DNA with at least 90%, 91%, 92%, 93%, 94%, 95%, 96%, 97%, 98%, 99% identity to the human Galectin-1 DNA that encodes a polypeptide that retains Galectin-1 activity on microglia. Alternatively, a DNA sequence variant can be described as hybridizing under stringent conditions to the human Galectin-1 DNA, or the complement thereof. The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing are 0.015 M sodium chloride, 0.0015 M sodium citrate at 65-68° C. or 0.015 M sodium chloride, 0.0015M sodium citrate, and 50% formamide at 42° C. See Sambrook et al., Molecular Cloning: A Laboratory Manual, 2nd Ed., Cold Spring Harbor Laboratory, (Cold Spring Harbor, N.Y. 1989).

Unless defined otherwise, all technical and scientific terms used herein have the same meaning as commonly understood by one of ordinary skill in the art to which this disclosure belongs. Any methods and materials similar or equivalent to those described herein can also be used in the practice or testing of the present disclosure.

All publications mentioned herein are incorporated herein by reference to disclose and describe the methods and/or materials for the purpose for which the publications are cited. To the extent the material incorporated by reference contradicts or is inconsistent with this specification, the specification will supersede any such material.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible. This disclosure is intended to provide support for all such combinations.

As used herein, “contemplated,” “may,” “may comprise,” “may be,” “can,” “can comprise” and “can be” all indicate something envisaged by the inventors that is functional and available as part of the subject matter provided.

### EXAMPLES

While the following examples describe products and methods of the disclosure, variations and modifications will occur to those skilled in the art. Accordingly, only such limitations as appear in the claims should be placed on the invention.

### Example 1

A series of experiments was performed to demonstrate that cell-secreted GAL1 reduces the inflammatory phenotype of M1 microglia in vitro.

### Extracellular Galectin-1 Reduces ALS Microglia-Mediated Motor Neuron Toxicity

GAL1 DNA or RFP DNA (control) was cloned into a mammalian expression vector and used to transfect HEK-293 cells. HEK-293 cells were maintained in Iscove's modified Dulbecco's media containing 10% FBS, 1% L-glutamine, and 1% penicillin/streptomycin. Upon reaching ˜60% confluence, HEK-293 cells were transfected with pBOB-Galectin1 or pBOB-RFP plasmids in Iscove's modified Dulbecco's media containing 10% FBS, 1% L-glutamine, and 1% penicillin/streptomycin. 24 hours post transfection, the medium was changed to Iscove's modified Dulbecco's media containing 2% FBS, 1% L-glutamine, and 1% penicillin/streptomycin. The supernatant was collected at every 24 hour interval for 3 days post transfection. Supernatant was filtered through 0.2μ filter and stored frozen at −80C. GAL1 ELISA confirmed the overexpression of GAL1 in pBOB-Galectin1 transfected cells as compared to pBOB-RFP transfected cells. Corresponding increase in the secreted GAL 1 was also found in supernatant from GAL1 transfected cells.

Adult microglia were isolated from brains of SOD1^{G93A} and WT mouse littermates as previously described [Moussaud and Draheim, J Neurosci Methods, 187 (2): 243-253 (2010)] with minor modifications. 4-month old SOD1^{G93A} and WT littermate mice were deeply anesthetized and perfused transcardially with ice-cold Ringers solution (Fisher Scientific). Brains that appeared to not be fully exsanguinated were discarded. Brains were fragmented with a scalpel and incubated with an enzymatic solution containing papain for 60 minutes at 37° C., 5% CO2. The papain solution was quenched with 20% FBS in HBSS and centrifuged for 4 minutes at 200 g. The pellet was resuspended in 2ml of 0.5 mg/ml DNase I (Worthington Biochemical) in HBSS and incubated for 5 min at room temperature. The brain tissue was gently disrupted with fire-polished Pasteur pipettes and then filtered through a 70 micron cell strainer (Fisher Scientific) and centrifuged at 200 g for 4 minutes. The resulting pellet was then resuspended in 20 ml of 20% isotonic Percoll (GE healthcare) in HBSS. 20 mL of pure HBSS was carefully laid on top the percoll layer and centrifugation was performed at 200 g for 20 min with slow acceleration and no brake. The interphase layer containing myelin and cell debris was discarded, and the pellet containing the mixed glial cell population was washed once with HBSS and suspended in Dulbecco's modified Eagle's/F12 medium with GlutaMAXTM (DMEM/F12) supplemented with 10% heat inactivated FBS, antibiotic-antimycotic (all from Life Technologies) and 5 ng/ml of carrier-free murine recombinant granulocyte and macrophage colony stimulating factor (GM-CSF) (R&D Systems). The cell suspension from four mouse brains was plated on a 15 cm2 plate (Corning) coated with poly-1-lysine (Sigma) and maintained in culture at 37° C. in a 95% air/5% CO2. The medium was replaced every 3 days until the cells reached confluency (after approximately 2 weeks). After the glial layer becomes confluent, microglia form a non-adherent, floating cell layer that can be collected, re-plated, and cultured for an extended period of time. After collecting the floating layer, microglia were incubated for 3 days without GM-CSF before re-plating for co-culture with motor neurons. Collected microglia were characterized by immunocytochemistry.

The microglia from end stage SOD1^{G93A} mice and age-matched wild type mice were treated with the HEK293 supernatant containing Galectin-1 or RFP for 3 days. The pre-conditioned microglia were then co-cultured with HB9:GFP+ motor neurons (MNs) as follows.

Hb9-GFP⁺ MNs were plated in 96-well plates coated with poly-ornithin (10 μg/ml, Sigma) and laminin (5 μg/ml, Invitrogen) at a density of 6,000 cells per well in 100 μl MN medium containing DMEM: F12 (Invitrogen), 5% horse serum, 2% N2 (Invitrogen), 2% B27 (Invitrogen)+GDNF (10 ng/ml, Invitrogen), BDNF (10 ng/ml, Invitrogen), and CNTF (10 ng/ml, Invitrogen). The day after, pre-conditioned microglia were plated on top of MNs at a density of 35,000 cells per well in 100 μl MN media. The co-culture plate was imaged each day by the IN Cell Analyzer 6000 (GE Healthcare). Images were processed and analyzed using IN Cell Developer Toolbox 1.9 and IN Cell Analyzer Workstation 3.7 software (GE Healthcare) to quantify number of surviving GFP+MNs per well. Depending on the assay, culture medium or cell lysates were prepared after 3 day co-culture.

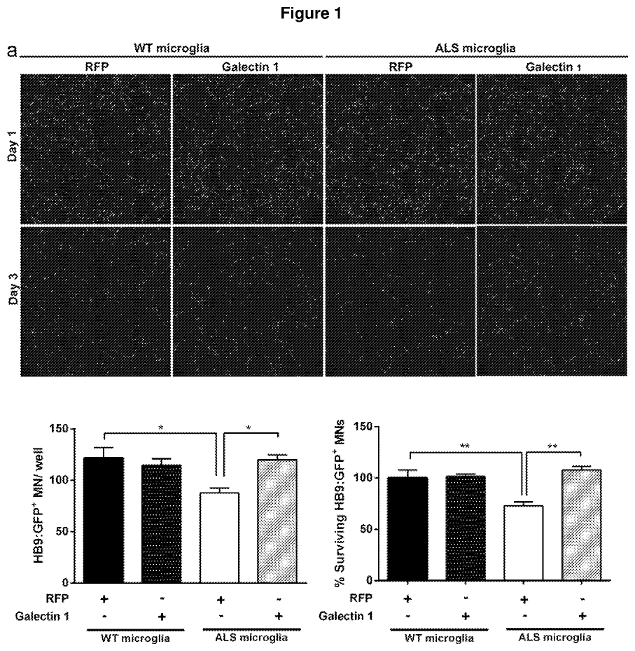

SOD1^{G93A} microglia treated with Galectin-1 containing medium were able to rescue motor neuron toxicity (FIG. 1).

Conditioning of ALS Microglia with Galectin-1 Results in Reduced Secretion of TNF-α

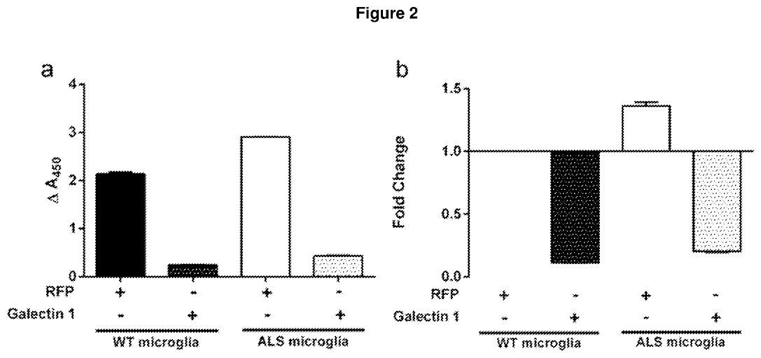

To demonstrate the enhanced motor neuron survival after GAL1 preconditioning of ALS microglia was due to the modulation of the inflammatory phenotype of these microglia, coculture supernatants were screened for TNF-α levels. ELISA-based quantification of TNF-α levels in the media collected from the RFP- or Galectin-1-conditioned wild type or SOD1^{G93A} microglia, co-cultured with HB9:GFP⁺ motor neurons showed Galectin-1 conditioning reduced the levels of TNF-α secreted from SOD1^{G93A} and wild-type microglia (FIG. 2).

Galectin-1 conditioning of ALS microglia affects M1 and M2 marker expression levels

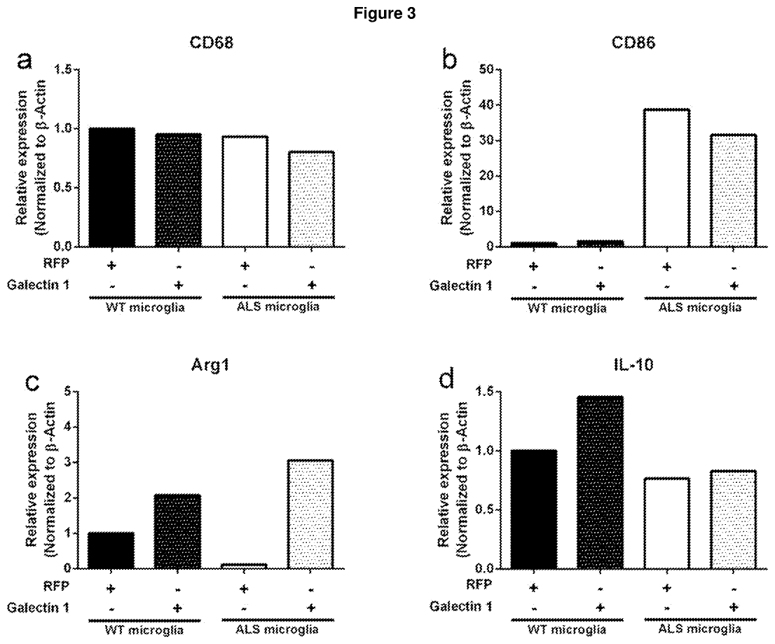

qRT-PCR based quantification of expression levels of CD68, CD86 (M1 markers) and Arginase 1, IL-10 (M2 markers) was performed on the RFP or Galectin-1 conditioned wild type or SOD1^{G93A} microglia, co-cultured with HB9:GFP⁺ motor neurons. Galectin-1 conditioning reduces the expression of neurotoxic M1 markers and enhances the expression of anti-inflammatory M2 markers in SOD1^{G93A} microglia. Although only modest changes in M1 marker expression were observed, Galectin-1 conditioning significantly increased Arginase 1 expression (M2 marker) in SOD1^{G93A} microglia. A similar trend was also observed with IL-10 expression (FIG. 3).

### Galectin-1 Conditioning Modulates NF-κB Activation in ALS Microglia

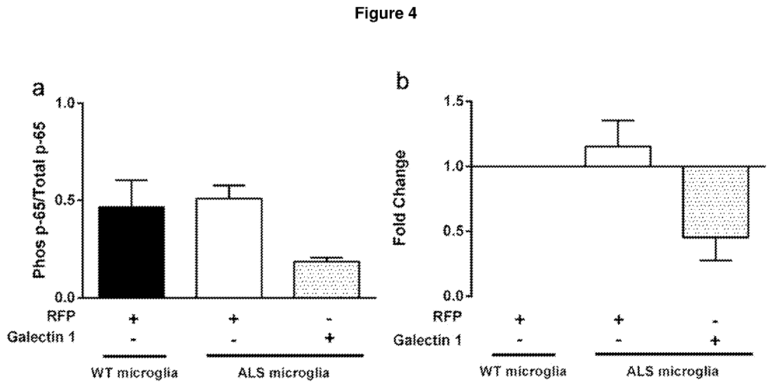

NF-κB activation in RFP-or Galectin-1-conditioned wild-type and SOD1^{G93A} microglia in co-culture with HB9:GFP⁺ motor neurons was determined by performing ELISA based quantification of phospho-p65 and Total p-65. Galectin-1 conditioning of SOD1^{G93A} microglia resulted in lower levels of phospho-p65/Total p-65 as compared to RFP treated SOD1^{G93A} and wild-type microglia, suggesting reduced NF-κB activation (FIG. 4).

### Summary

This in vitro data indicates that GAL1-mediated deactivation of ALS microglia results in enhanced motor neuron survival.

### Example 2

A series of experiments was performed to demonstrate that cell-secreted GAL1 reduces the inflammatory phenotype of M1 microglia in vivo.

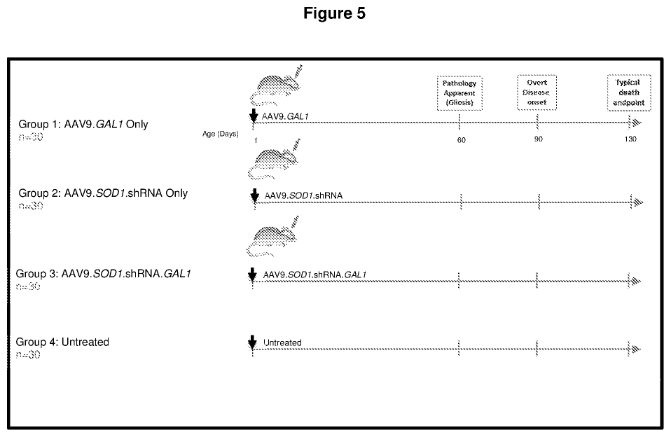

Mice expressing the SOD1^{G93A} mutation were assigned to one of four treatment groups: AAV9.GAL1 only, AAV9.SOD1.shRNA only, AAV9.SOD1.shRNA.GAL1, or untreated controls (FIG. 5).

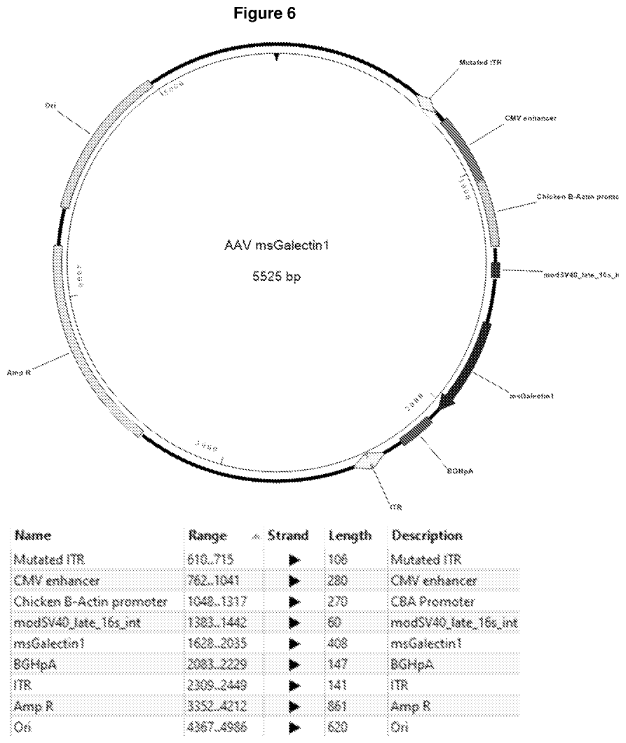

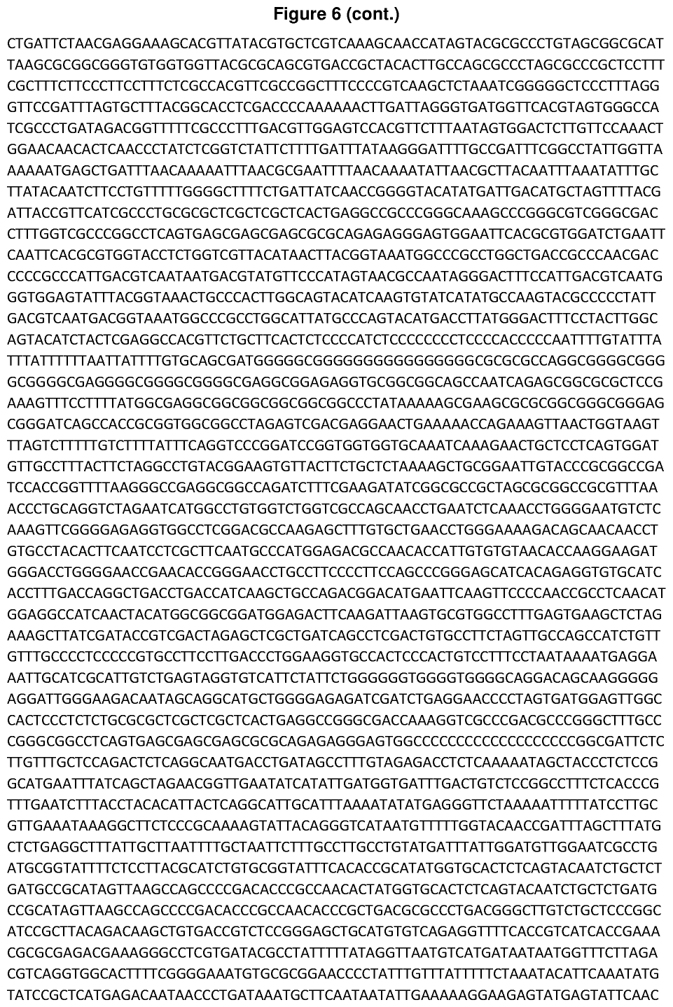

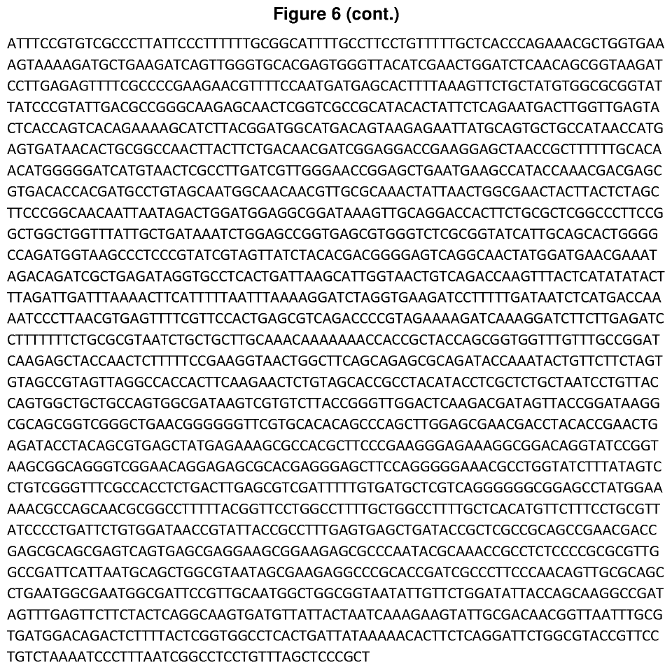

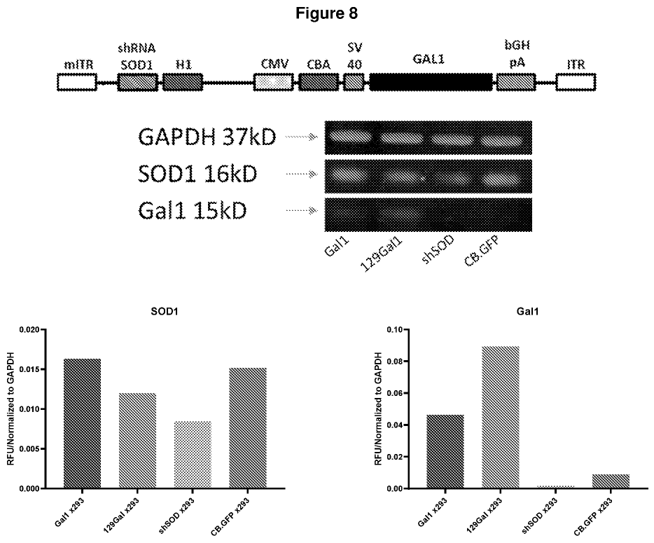

The AAV9.GAL1 vector (also referred to as AAV MS Galectin-1 vector) contained mouse GAL1 cDNA expressed under the control of the chicken β-Actin promoter, leading to high expression levels in astrocytes and motor neurons. The plasmid used to produce the AAV is shown in FIG. 6. The AAV9. SOD1.shRNA vector has been described previously in Foust et al., Mol. Ther., 21:2148-2159 (2013) and Iannitti et al., Mol. Ther. Nucleic Acid, 12:75-88 (2019) In the vector, an shRNA construct targeting human SOD1 is expressed under the control of the H1 promoter. The vector also contained a stuffer sequence optimized for efficient packaging of the AAV vector. The AAV9.SOD1.shRNA.GAL1 vector contained SOD1.shRNA expressed under the control of the H1 promoter and GAL1 cDNA expressed under the control of the chicken β-Actin promoter. The plasmid used to produce the AAV is shown in FIG. 7. Self-complementary AAV were produced by transient transfection procedures using each plasmid, along with a plasmid encoding Rep2Cap9 sequence as previously described along with an adenoviral helper plasmid pHelper (Stratagene, Santa Clara, CA) in 293 cells. The same SOD1.shRNA and SOD1.shRNA.GAL1 constructs were also packaged in a PHP.B capsid. The PHP.B serotype has been shown to increase transduction in neurons of adult mice as compared to AAV9. Transfection of HEK293 cells showed the plasmids and scAAV worked as designed. For example, AAV.SOD1.shRNA.Gal1 reduced SOD1 expression and increased Gall expression in transfected HEK293s. HEK-293 cells were maintained in Dulbecco's Modified Eagle Medium containing 10% FBS, 1% L-glutamine, and 1% penicillin/streptomycin. Upon reaching ˜80% confluence, cells were transfected with plasmids AAV.SOD1.shRNA.Gal1, AAV.SOD1.shRNA, AAV.Gal1 and AAV.GFP. Protein lysates were prepared 72 hours post-transfection and analyzed for SOD1 and Gal1 levels by western blot (FIG. 8).

Thirty mice were utilized for each treatment group. All procedures were performed in accordance with the NIH Guidelines and approved by the Abigail Wexner Research Institute at Nationwide Children's Hospital (Columbus, OH). High-copy SOD1^{G93A} mice were obtained from Jackson Laboratories (Bar Harbor, ME) and bred. Animals were genotyped before the treatment to obtain SOD1^{G93A}-expressing mice and their wild-type littermates. Both male and female mice were included in the SOD1^{G93A} and wild-type mouse experiments. To obtain statistically meaningful results for behavior and survival analysis, a power analysis was performed. The analysis showed a minimum of 8 animals per sex per group were required. To meet this requirement, group sizes were set to at least 10 males and 10 females per treatment group to allow for eventual random non-related health issues of mice without losing statistical power. An additional 5 males and 5 females per treatment group were added for immunohistochemical and expression analysis at various time points.

Mice received either AAV9.GAL1, AAV9.SOD1.shRNA, or AAV9.SOD1.shRNA.GAL1. For intracerebroventricular (ICV) injections of mice at P1, the pups were anesthetized on ice for 10 minutes prior to injection. Injection was performed with 30G Hamilton syringes as previously described60. The rAAV9 were diluted in phosphate-buffered saline to obtain correct doses. The total volume injected for each animal was <5 μl. The dose, 5×10¹⁰ vector genomes/animal, allowed widespread targeting of cells throughout the entire brain and spinal cord. For intrathecal (IT) injections, adult mice were anesthetized with continuous isofluorane and lumbar injection was performed with 30G Hamilton syringes and a syringe pump. In brief, the needle was inserted between the L5/L6 vertebral discs and the vector was injected at the rate of 3 μL/min. A dose of 5×10¹⁰ vg/mouse was used for PHP.B vectors and a dose of 1.65×10¹¹ vg/mouse was used for AAV9 vectors in a total volume of 15 μL.

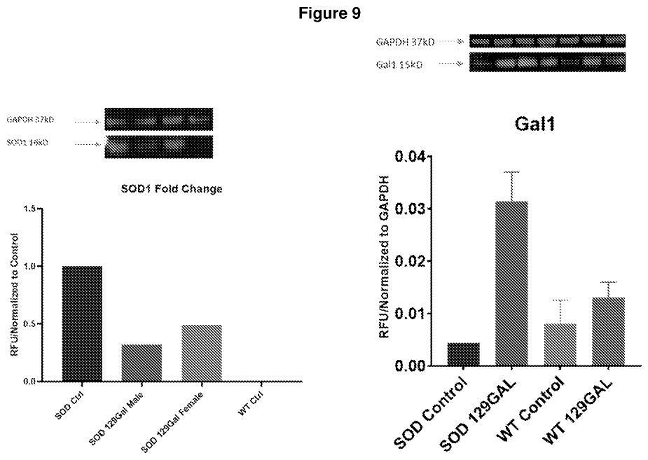

Consistent with the HEK293 cell expression assays, AAV.SOD1.shRNA.Gal1increased Gal1 expression in WT/SOD1^{G93A} mice and reduced SOD1 expression in SOD1^{G93A} mice. WT and SOD1^{G93A} mice were injected with AAV9.SOD1.shRNA.Gal1 vector intracerebroventricularly at P1. At 1 month post injection, the animals were sacrificed and the lumbar spinal cord tissues were harvested for analysis of SOD1 and Gal1 levels by western blot analysis (FIG. 9).

Intracerebroventricular delivery of AAV9.SOD1.shRNA.msGal1 improves survival in SOD1^{G93A} mice over AAV9.SOD1.shRNA treatment alone

The neonatal SOD1^{G93A} mice that received a single ICV injection of AAV9.msGal1, AAV9.SOD1.shRNA, or AAV9.SOD1.shRNA.msGal1 as described above were monitored until endpoint and compared with controls. Survival analysis was performed using Kaplan-Meier survival analysis. End stage was defined as an artificial death point when animals can no longer “right” themselves within 20 seconds after being placed on their back and/or has severe urinary incontinence and scalding. Onset and disease progression was determined from retrospective analysis of the data. Disease onset was defined as the age at which the animal reached its peak weight. Disease duration was defined as the period between disease onset and end stage.

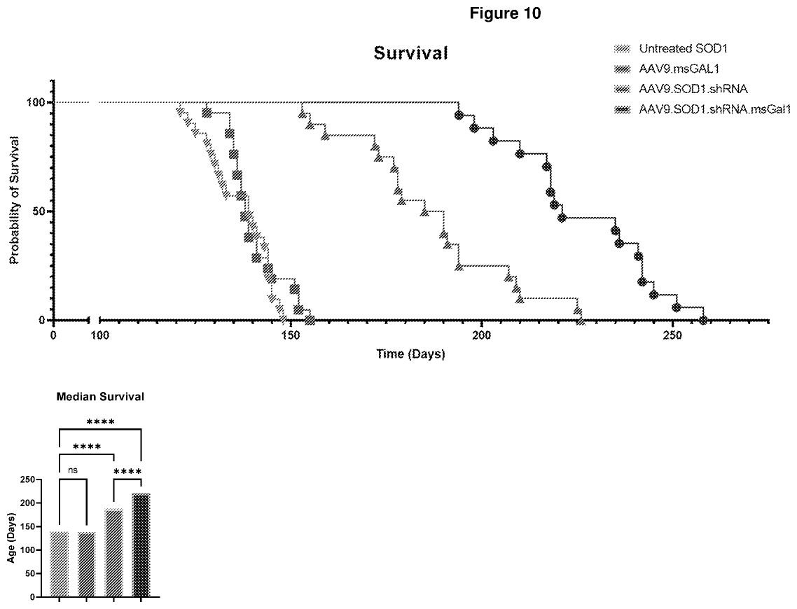

AAV9.SOD1.shRNA.msGal1 treatment significantly extended median survival of SOD1^{G93A} mice compared to AAV9.SOD1.shRNA, AAV9.msGal1, and uninjected controls (control, n=21, 139 days; AAV9.msGal1, n=21, 138 days; AAV9.SOD1.shRNA, n=20, 187.5 days; AAV9.SOD1.shRNA.msGal1, n=17, 221 days; one-way ANOVA, P<0.0001) (FIG. 10)

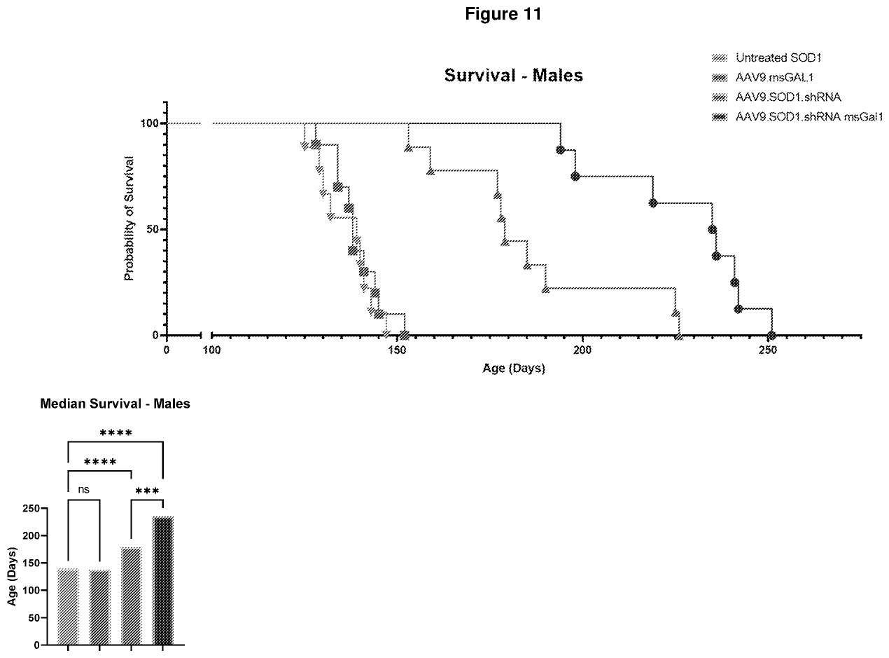

AAV9.SOD1.shRNA.msGal1 treatment significantly extended median survival of SOD1^{G93A} male mice compared to AAV9.SOD1.shRNA, AAV9.msGal1, and uninjected controls (control, n=9, 139 days; AAV9.msGal1, n=10, 138 days; AAV9.SOD1.shRNA, n=9, 179 days; AAV9.SOD1.shRNA.msGal1, n=8, 235.5 days; one-way ANOVA, P<0.0001) (FIG. 11).

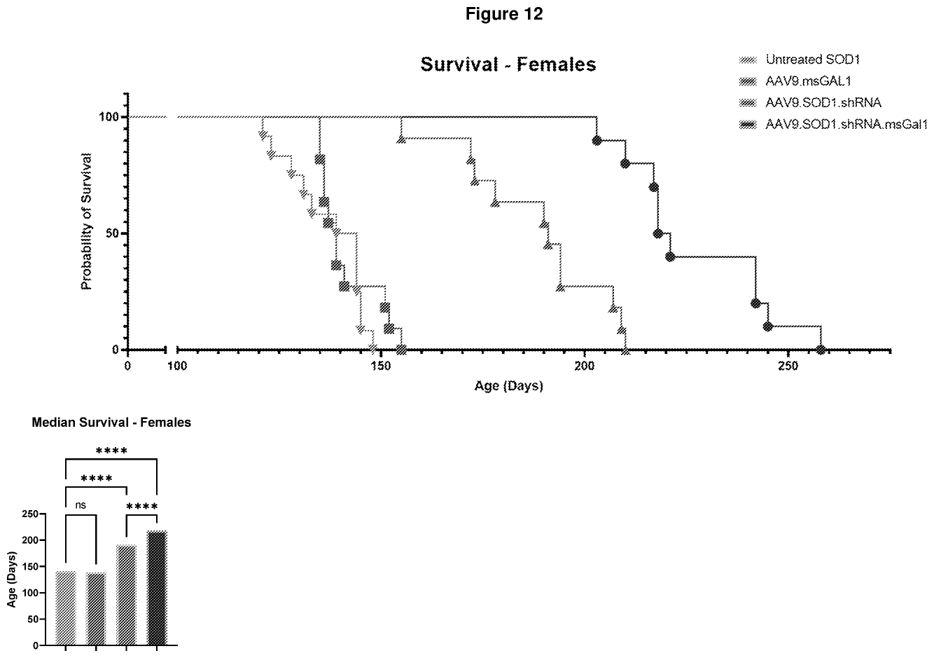

AAV9.SOD1.shRNA.msGal1 treatment significantly extended median survival of SOD1^{G93A} female mice compared to AAV9.SOD1.shRNA, AAV9.msGal1, and uninjected controls (control, n=12, 141.5 days; AAV9.msGal1, n=11, 139 days; AAV9.SOD1.shRNA, n=11, 191 days; AAV9.SOD1.shRNA.msGal1, n=9, 218 days; one-way ANOVA, P<0.0001) (FIG. 12).

Intracerebroventricular Delivery of AAV9.SOD1.shRNA.msGal1 and AAV9.SOD1.shRNA Improves Motor Performance in SOD1^{G93A} Mice

Treated and control SOD1^{G93A} mice as well wild-type littermates were monitored for changes in body mass twice a week. Motor coordination was recorded using an accelerating rotarod instrument (Columbus Instruments, Columbus, OH). Each bi-weekly session consisted of three trials on the accelerating rotarod beginning at 5 rpm/minute. The time each mouse remains on the rod was registered. Both SOD1^{G93A} and wild-type mice were subjected to bi-weekly assessment of forelimb and hindlimb grip strength using a grip strength meter (Columbus Instruments). Each bi-weekly session consisted of three tests per animal.

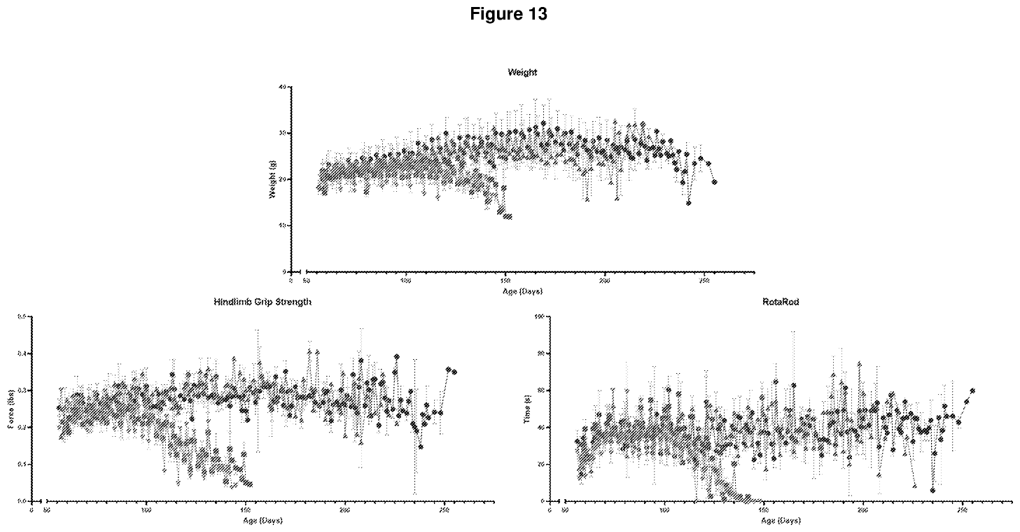

Animals treated with AAV9.SOD1.shRNA.msGal1 and AAV9.SOD1.shRNA had better maintained body weights and improved hindlimb grip strength and rotarod performance as compared with age-matched controls (FIG. 13). This indicates AAV9.SOD1.shRNA.msGal1 (n=20) and AAV9.SOD1.shRNA (n=21) treated animals maintained muscle tone and motor performance compared to AAV9.msGal1 (n=21) and uninjected controls (n=24).

Intrathecal Delivery of PHP.B.SOD1.shRNA.msGal1 Improves Survival in SOD1^{G93A} Mice

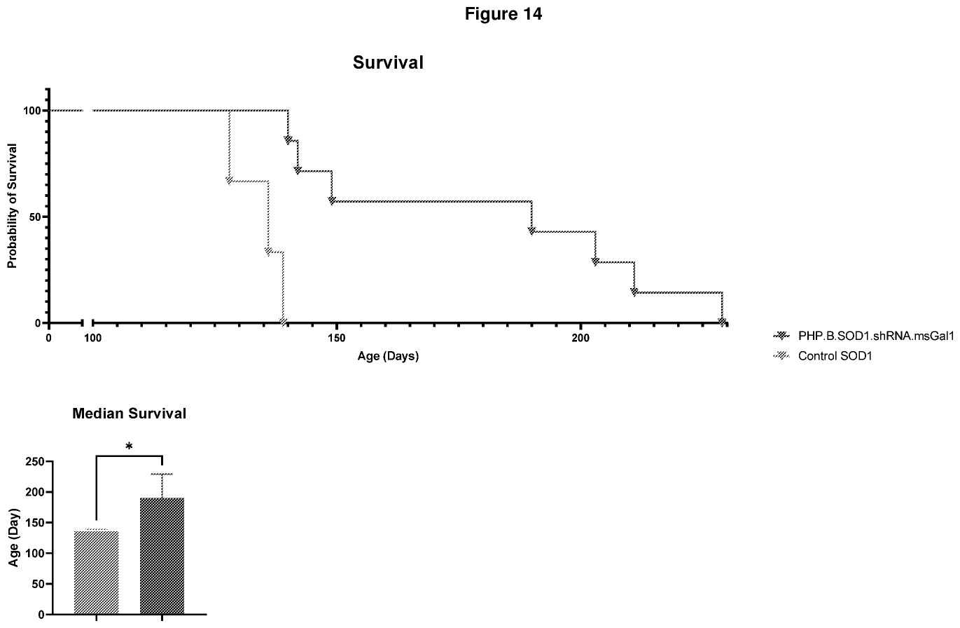

Adult SOD1^{G93A} mice (aged p80-p95) received a single intrathecal (IT) injection of PHP.B.SOD1.shRNA.msGal1. Treated mice were monitored until endpoint and compared with vehicle control mice. PHP.B.SOD1.shRNA.msGal1 injection significantly extended median survival of SOD1^{G93A} mice versus vehicle control (control, n=3, 136 days; PHP.B.SOD1.shRNA.msGal1, n=7, 190 days; log-rank test, P<0.05.) (FIG. 14).

Intrathecal Delivery of PHP.B.SOD1.shRNA.msGal1 Improves Body Weight in SOD1^{G93A} Mice

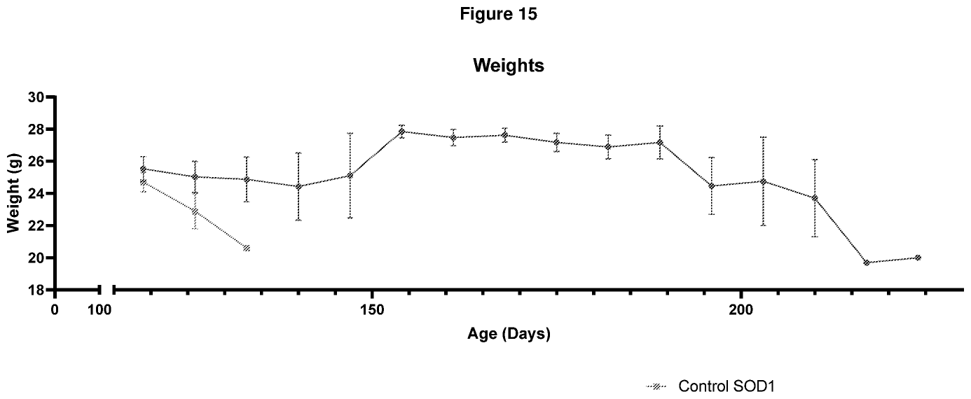

Adult SOD1^{G93A} mice (aged p80-p95) received a single IT injection of PHP.B.SOD1.shRNA.msGal1. PHP.B.SOD1.shRNA.msGal1 treated SOD1^{G93A} mice had maintained body weight versus vehicle control (n=3; PHP.B.SOD1.shRNA.msGal1, n=7), possibly indicating retained muscle tone in treated animals (FIG. 15).

### Summary

Mice treated with the SOD1 shRNA and GAL1 combination exhibited significantly increased motor function and survival in comparison to GAL1 only, SOD1 shRNA only, or untreated groups.

### Example 3

A series of experiments were performed to demonstrate the expression of Galectin-1 itself in patient-derived astrocytes or neurons is also neuroprotective.

Skin fibroblasts from multiple patients with various neurological and neurodegenerative disorders were directly reprogramed into Neural Progenitor Cells (NPCs) and further differentiated into induced astrocytes (iAs) according to methods described in Dennys et al. (2014). DOI: 10.3791/62016-v and Meyer et al., Proc. Natl. Acad. Sci. USA, 111:829-832 (2014). iAs were treated with AAV9.Gal1 (generated using the plasmid shown in FIG. 6) and then cocultured with mouse GFP neurons for 3 days to determine the effect of astrocytes on neuronal survival and morphology. Untreated iAs were utilized as controls.

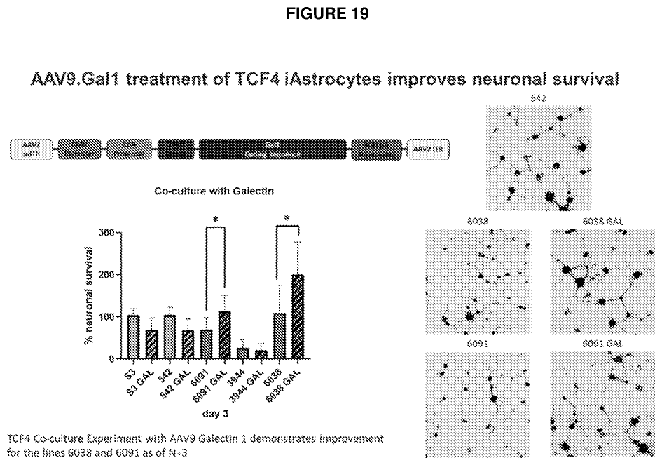

AAV9.Gal1 treatment of iAstrocytes derived from three patients with Pitt-Hopkins syndrome carrying different TCF4 mutations shows improvement in neuronal survival in 2 out of 3 patient lines tested (FIG. 19).

To demonstrate the effect of Galectin-1 expression in neurons, skin fibroblasts from patients with various neurological and neurodegenerative disorders were directly converted to induced neurons (iNs) according to the methods described in Ray et al., Cell Rep., 41:111751 (2022) and Sierra-Delgado et al., Biology (Basel), 12(6): 867 (2023). iNs were treated with AAV9.Gal1 (generated using the plasmid shown in FIG. 6) to determine its effect on conversion rate as well as neuronal morphology.

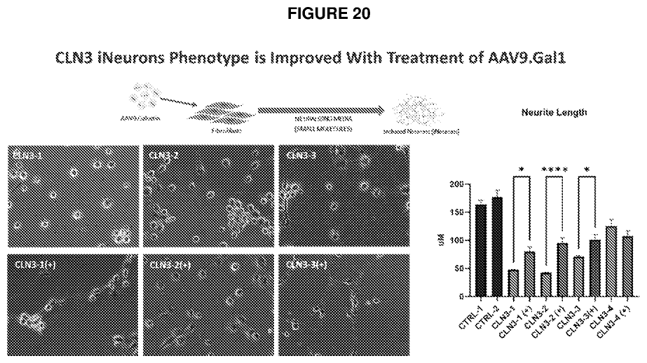

AAV9.Gal1 treatment of iNeurons derived from four patients with Batten CLN3 disease demonstrated significant improvement in neurite lengths for 3 out of 4 patient lines tested (FIG. 20).

### Example 4

A series of experiments were performed to demonstrate the effect of Galectin-1 in combination with other therapeutic proteins of interest. The present disclosure contemplates that targeting of astrocytes and neurons with AAV9, to deliver a healthy copy of a gene expressing a therapeutic protein of interest while also expressing Galectin-1 in these cells types, exerts positive intrinsic effects on these cell types while at the same time providing extrinsic neuromodulatory effects on the surrounding microglia.

In earlier Examples herein, Galectin-1 was expressed under an RNA PolII promoter with separate expression of an shRNA expression cassette or another transgene expression cassette. In this Example, Galectin-1 is expressed as a fusion protein with the therapeutic protein of interest using a 2A self-cleaving peptide sequence. Examples of self-cleaving peptide sequences are the following.

| Name | Sequence |
|---|---|
| T2A | (GSG) EGRGSLLTCGDVEENPGP (SEQ ID NO: 39) |
| P2A | (GSG) ATNFSLLKQAGDVEENPGP (SEQ ID NO: 40) |
| E2A | (GSG) QCTNYALLKLAGDVESNPGP (SEQ ID NO: 41) |
| F2A | (GSG) VKQTLNFDLLKLAGDVESNPGP (SEQ ID NO: 42) |

The therapeutic protein and Galectin are expressed from the same RNA PolII promoter which results in a fusion protein comprising gene product of interest-2A-Galectin-1 which is cleaved post-translation.

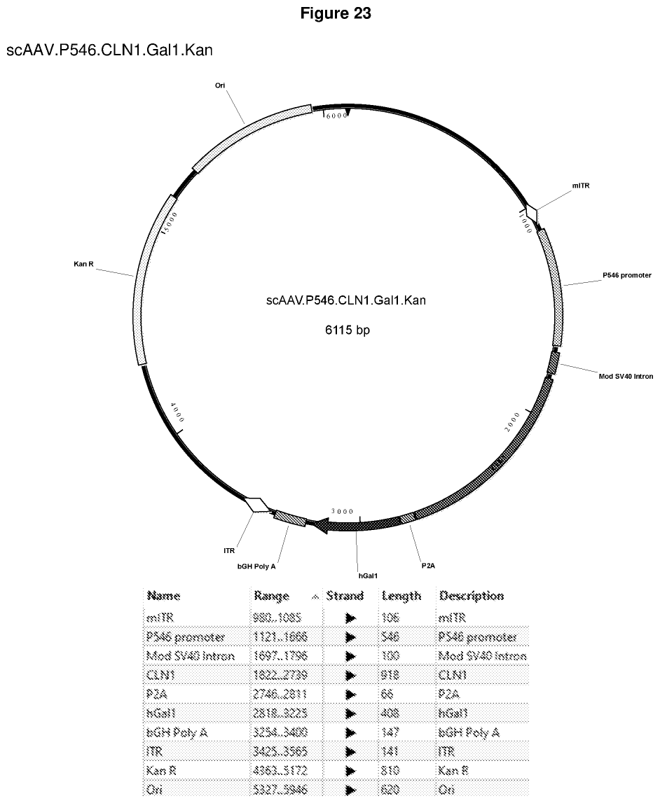

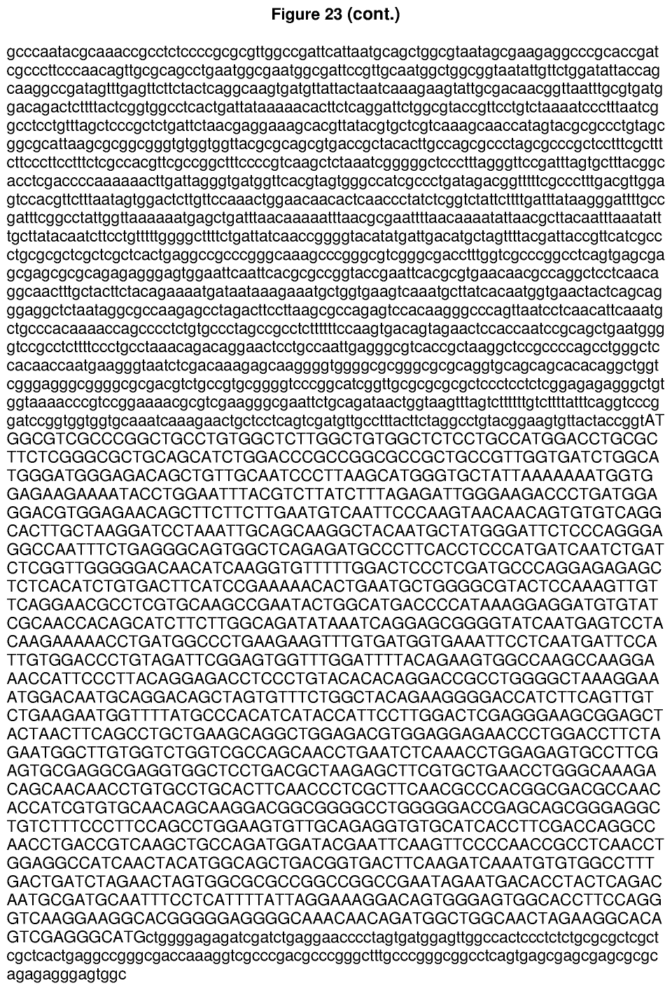

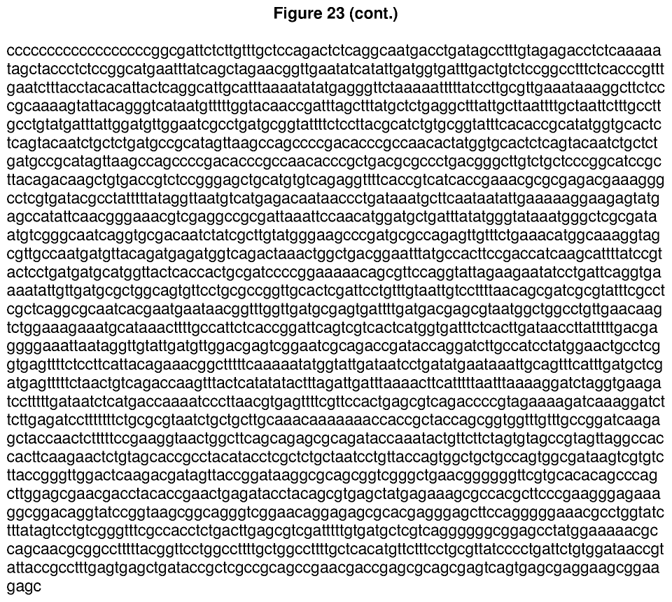

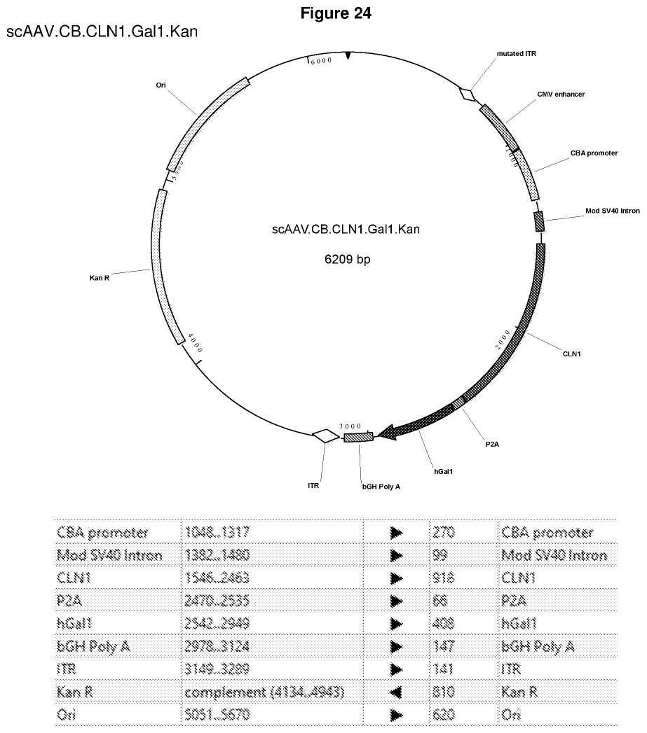

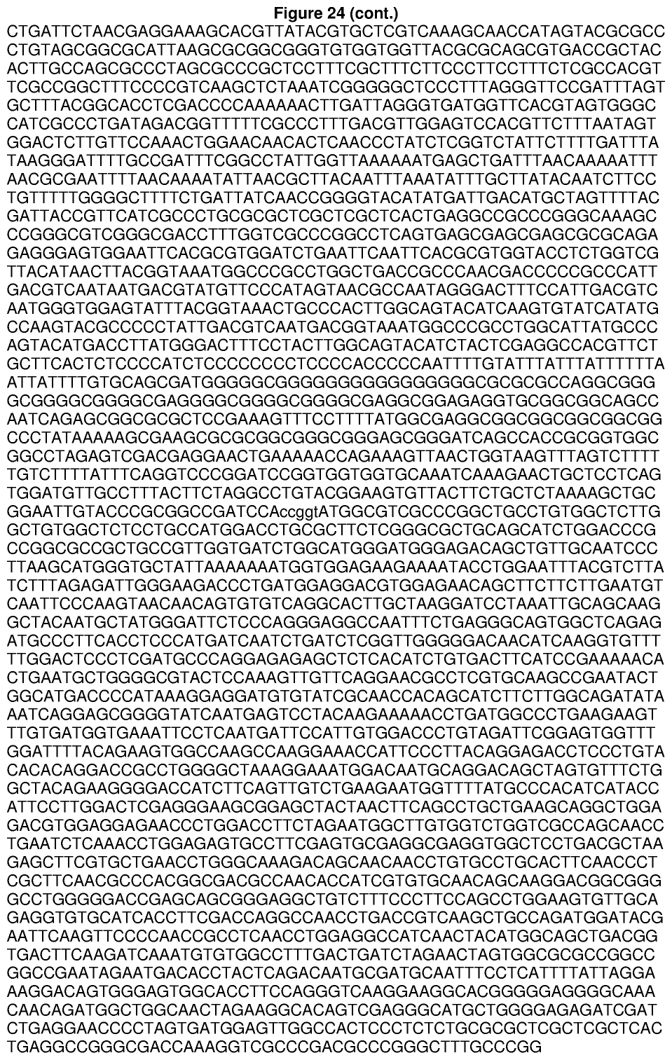

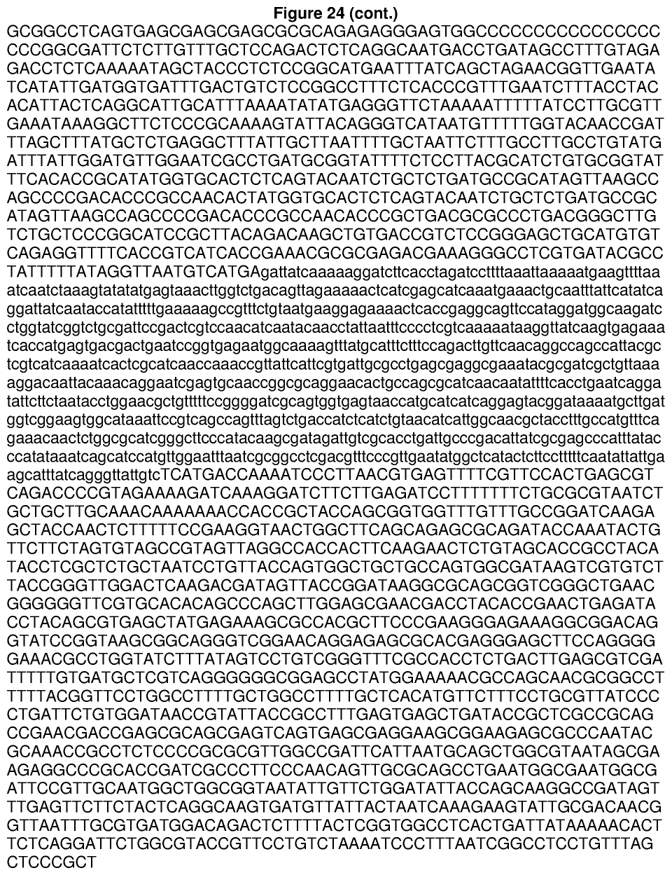

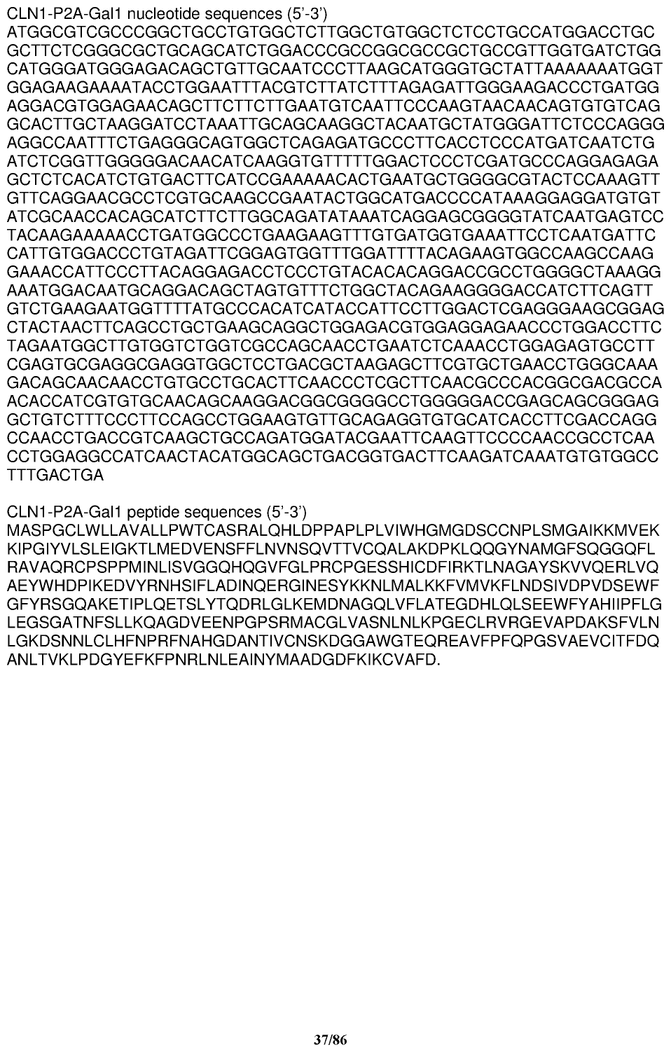

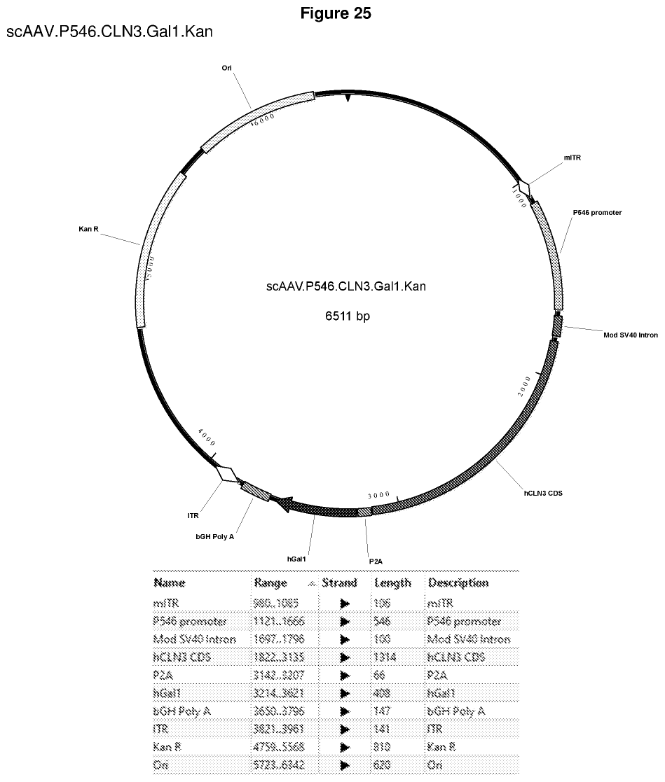

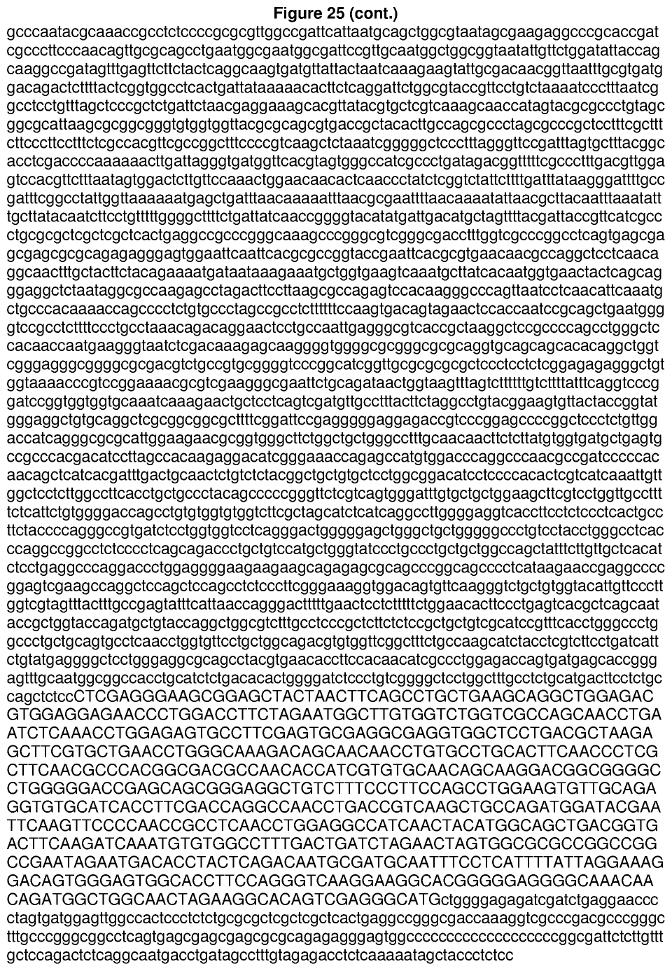

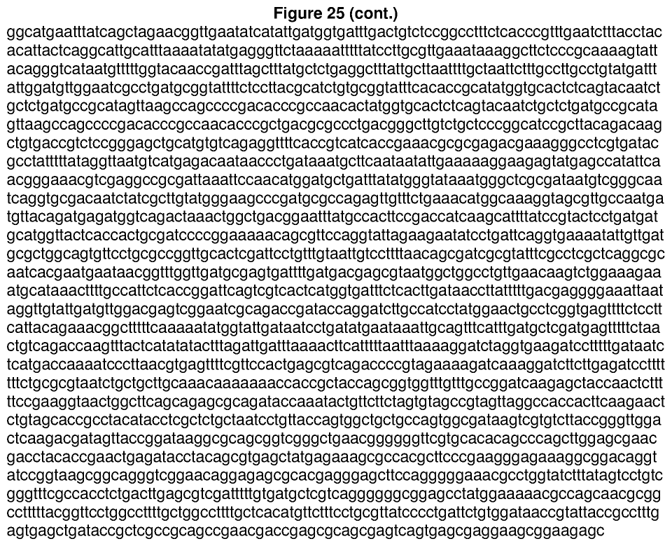

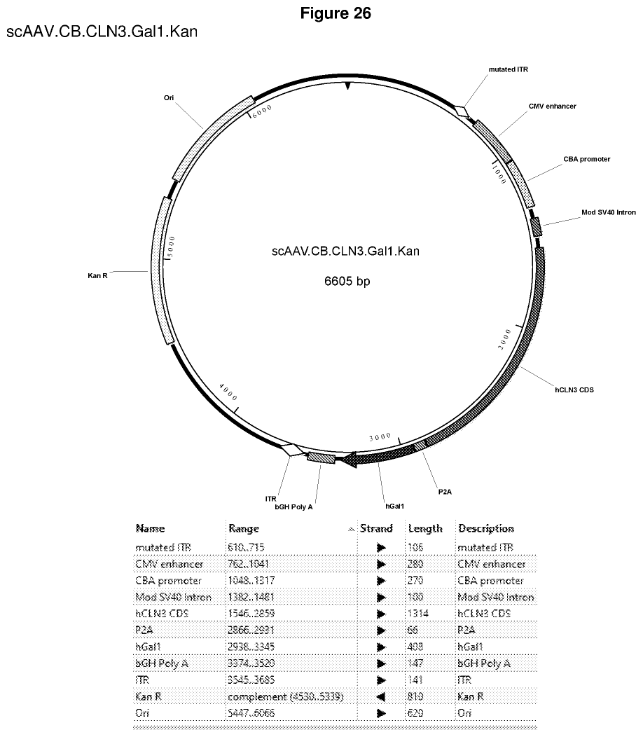

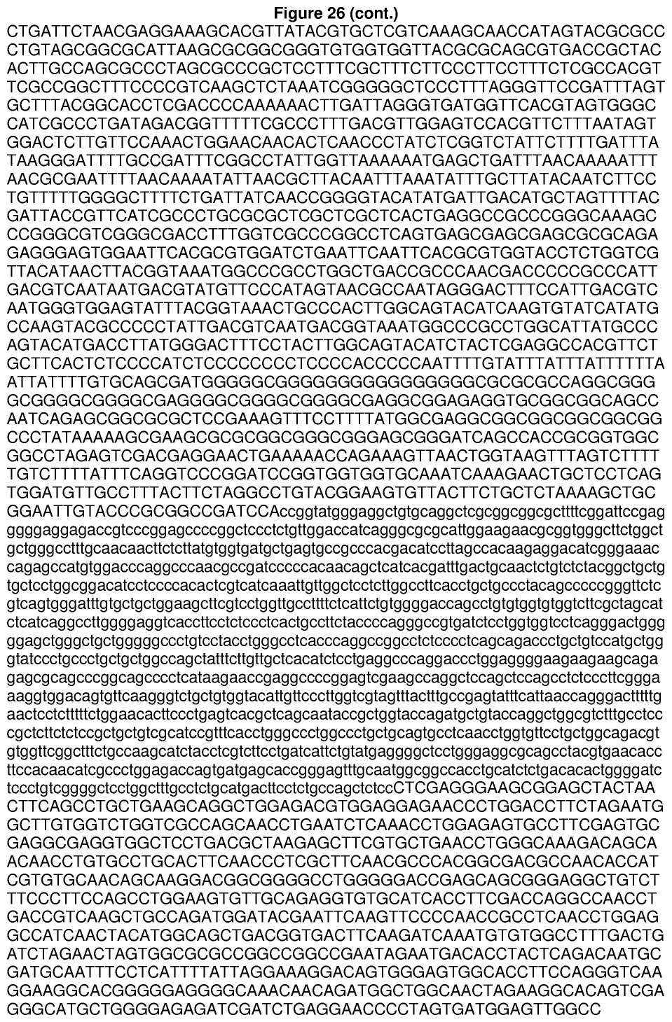

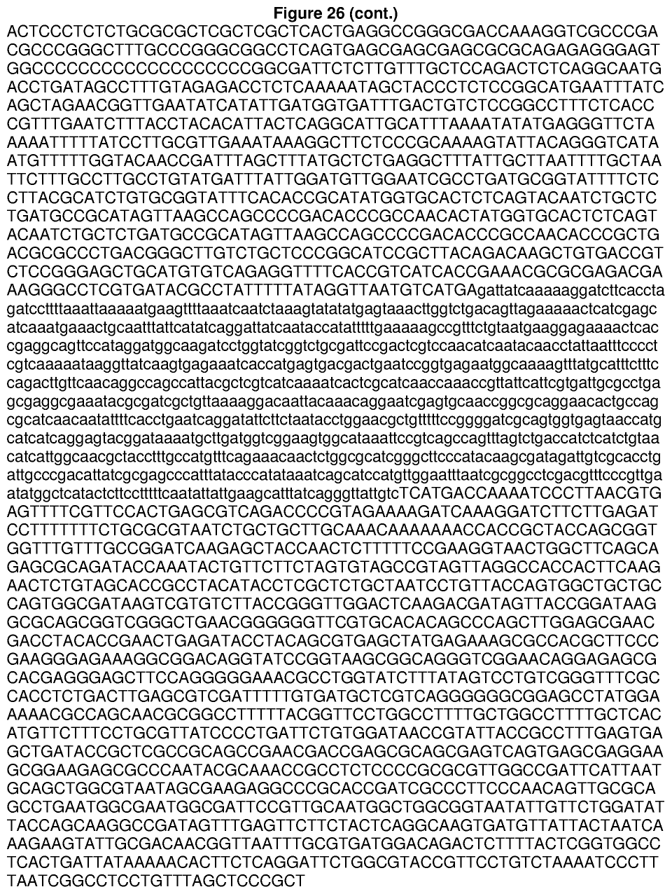

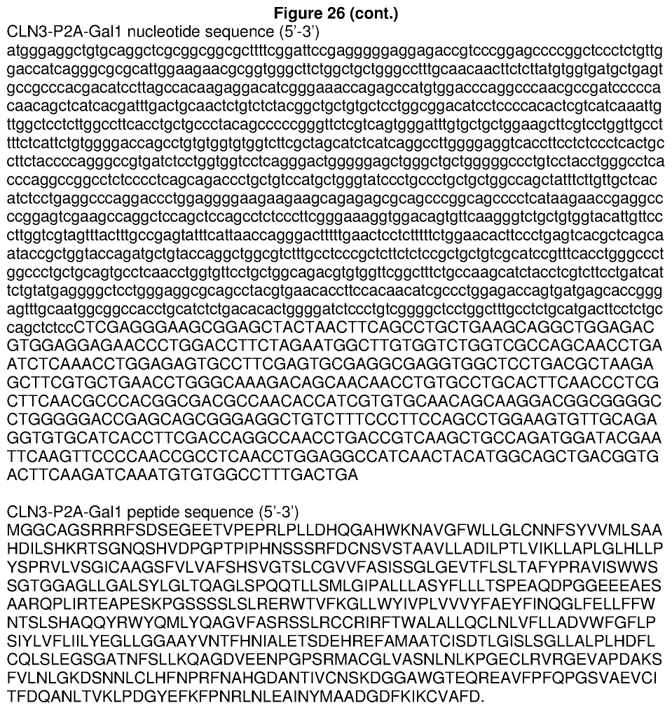

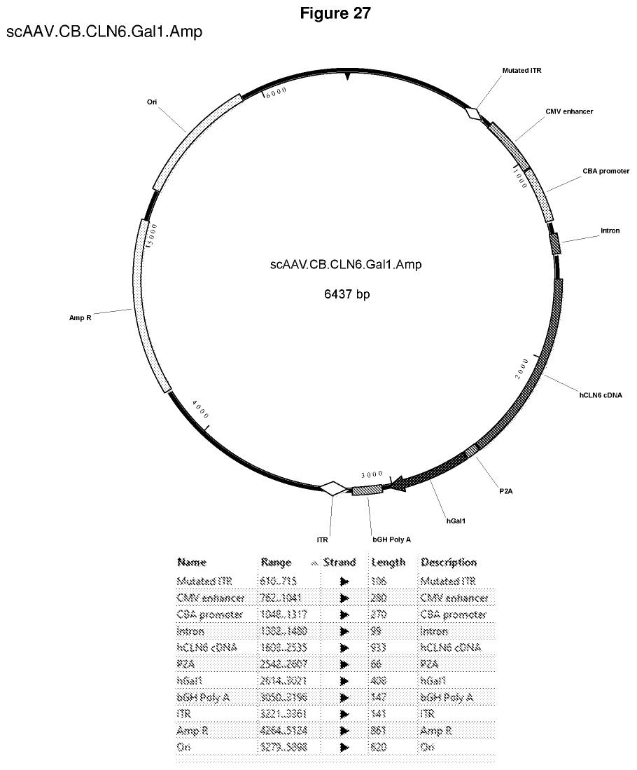

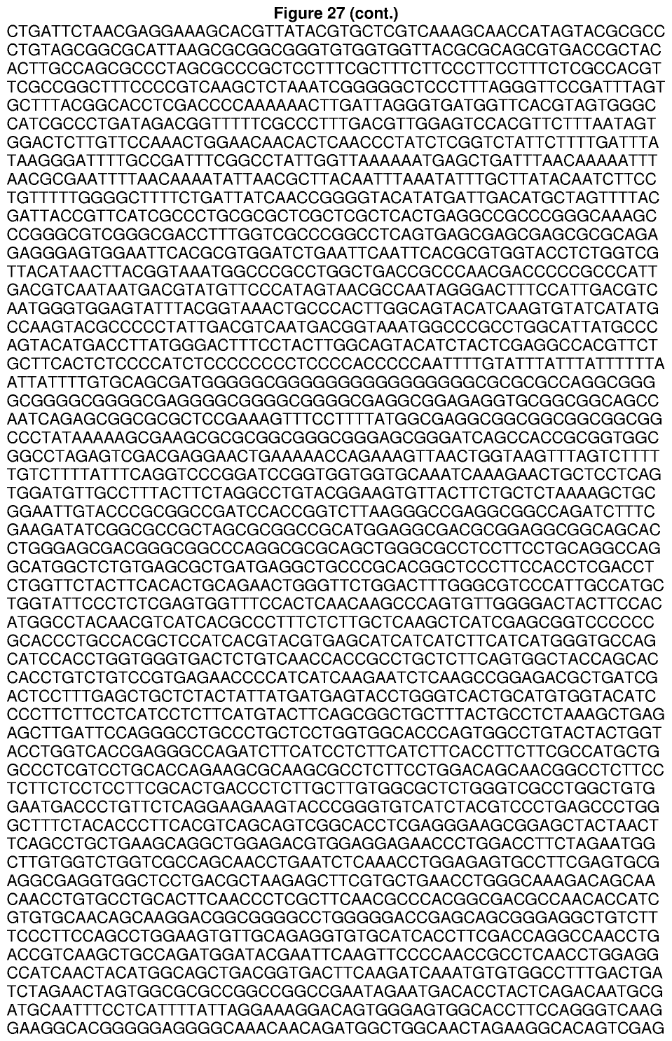

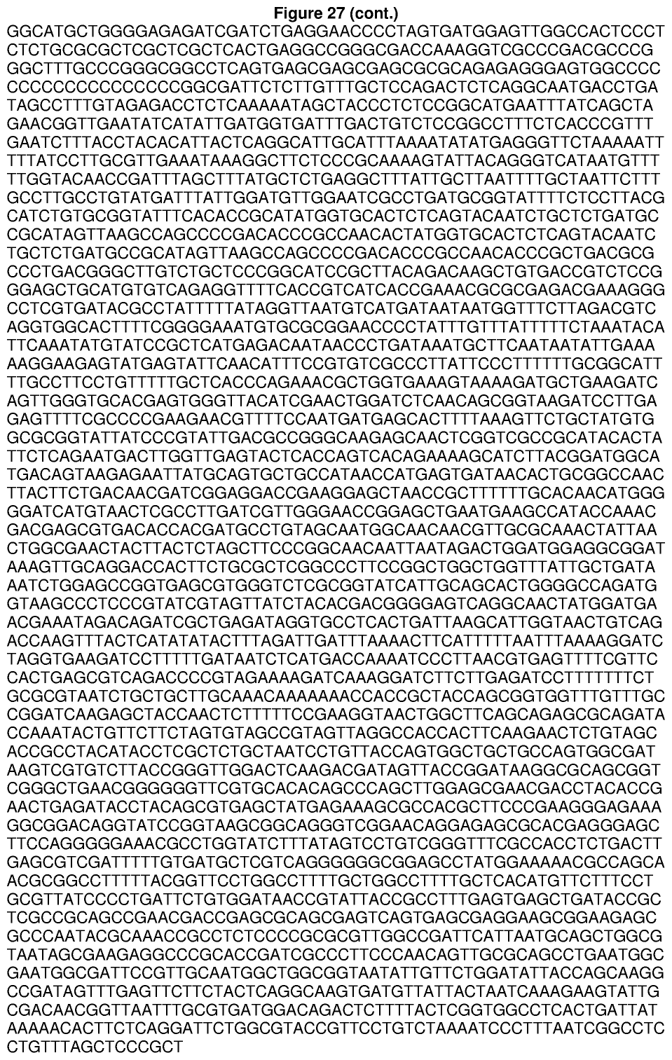

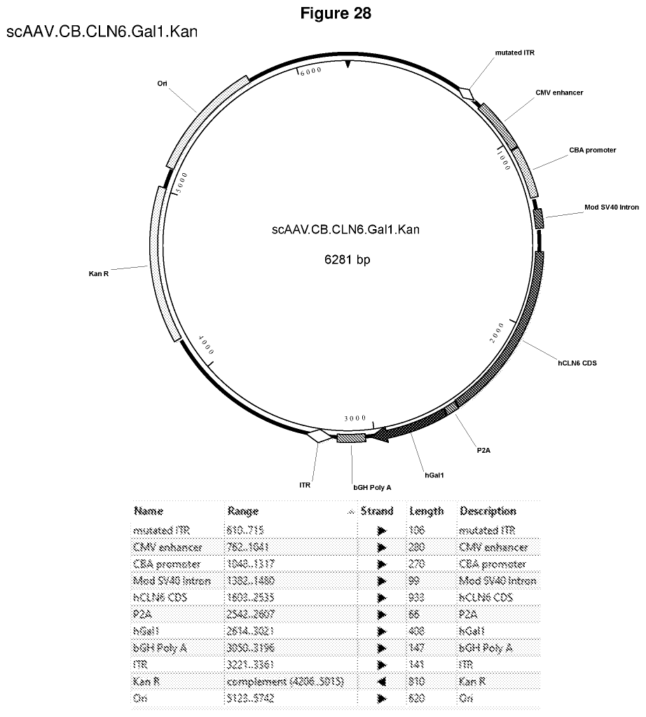

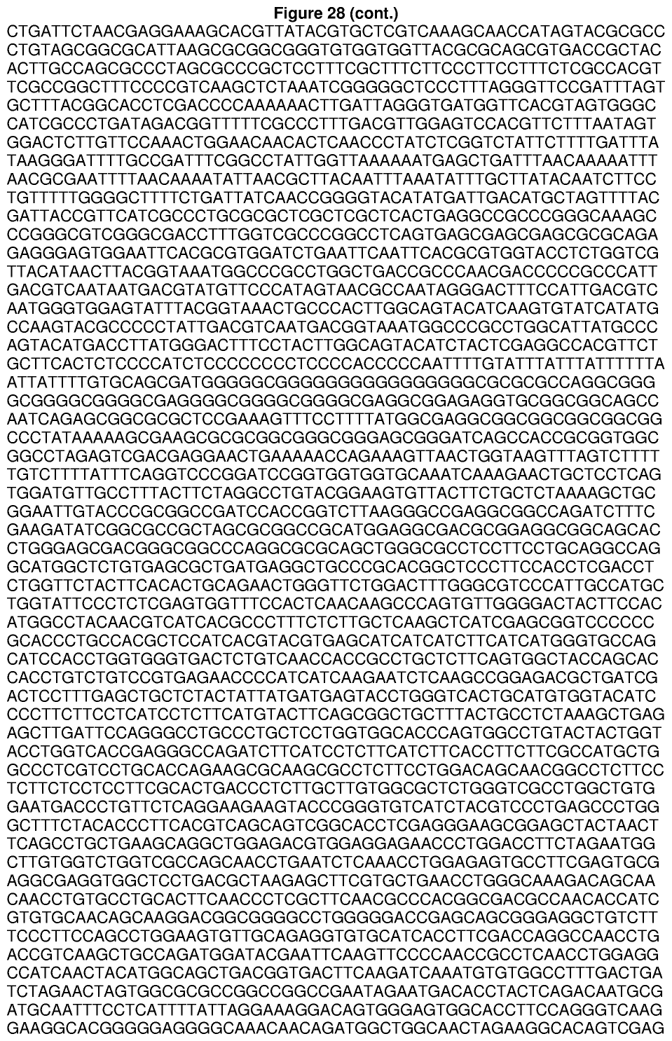

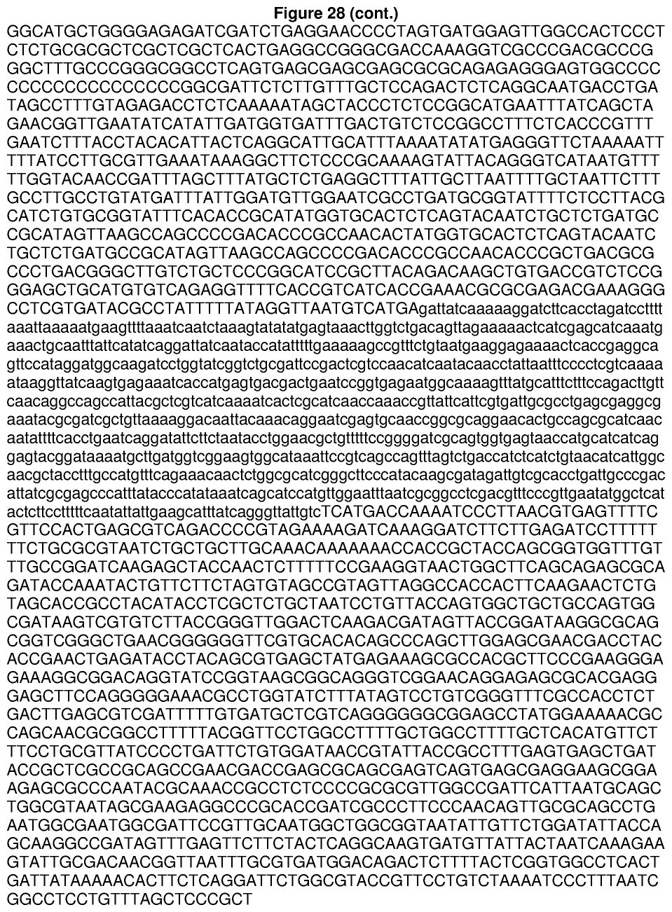

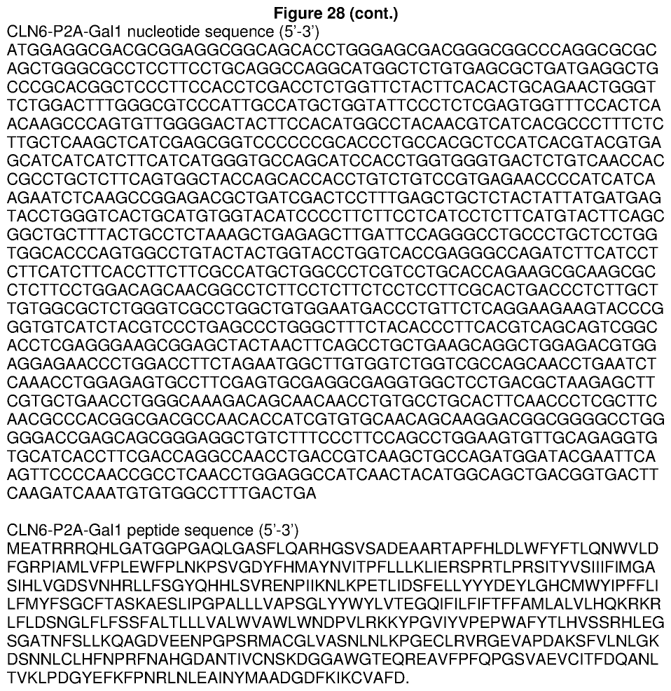

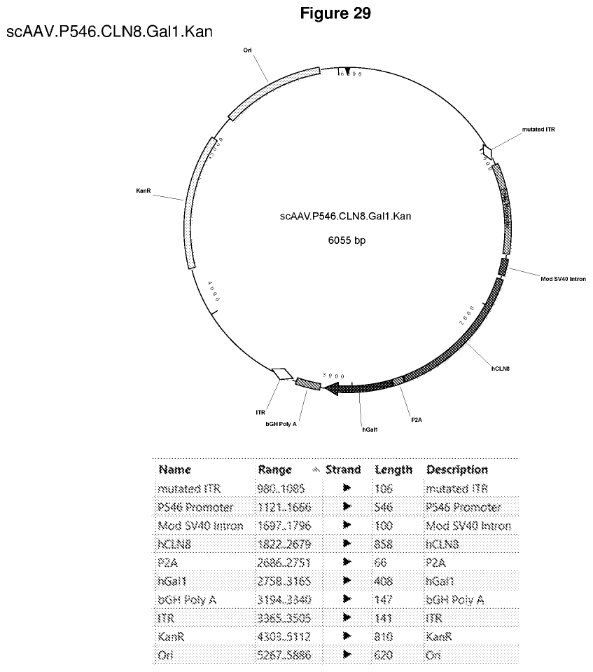

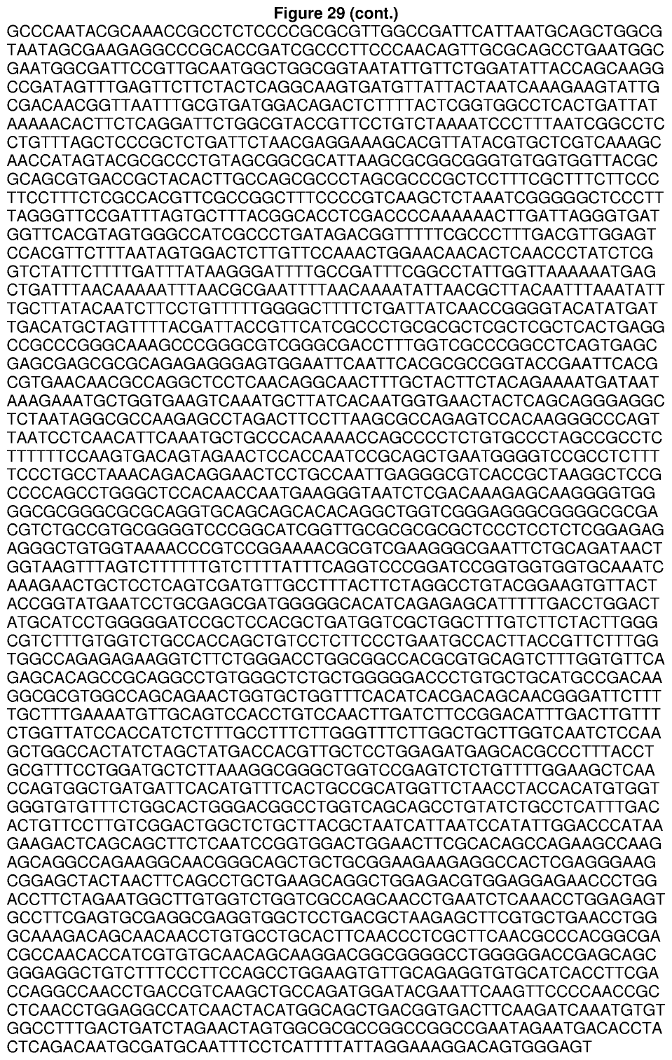

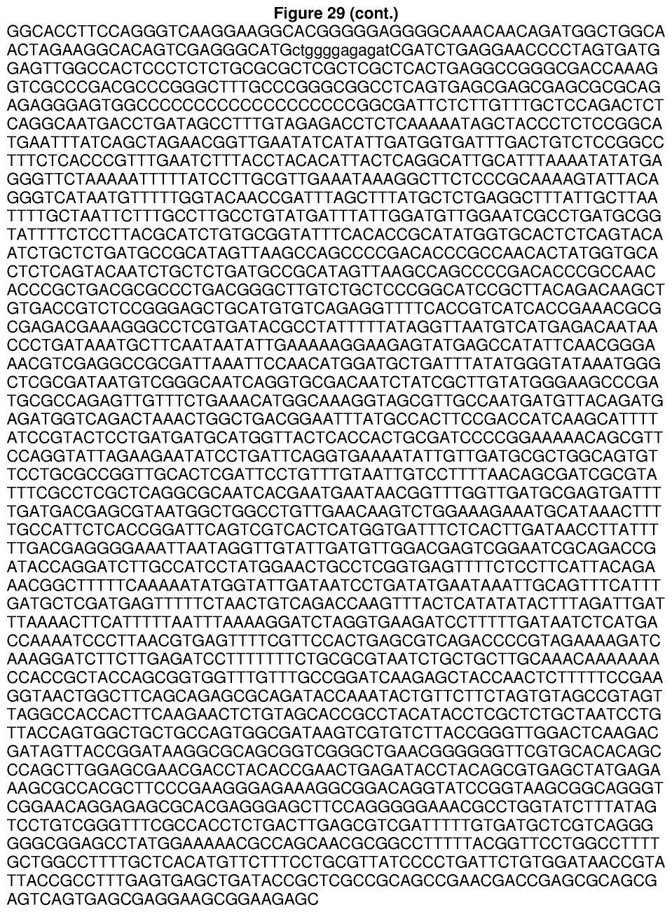

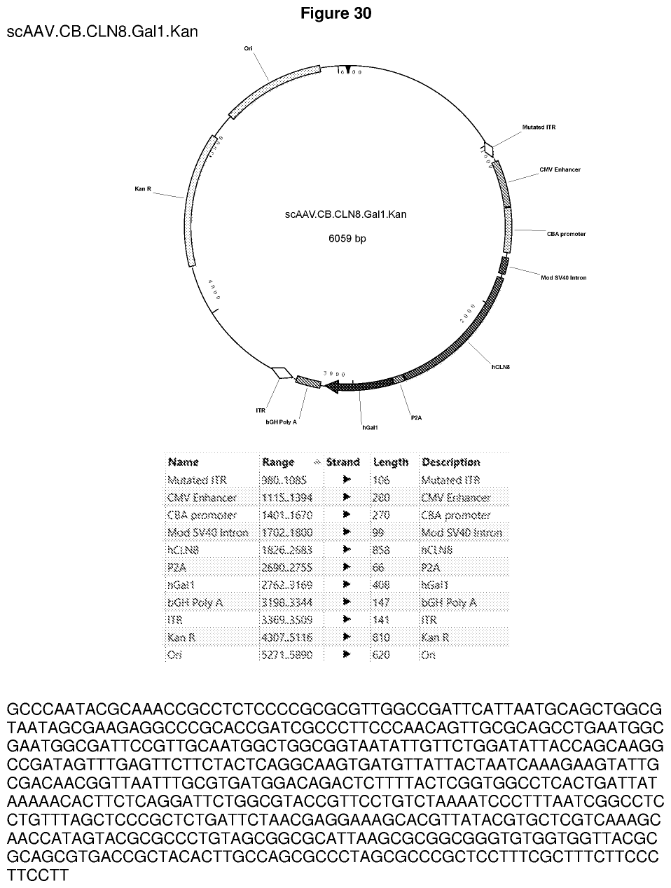

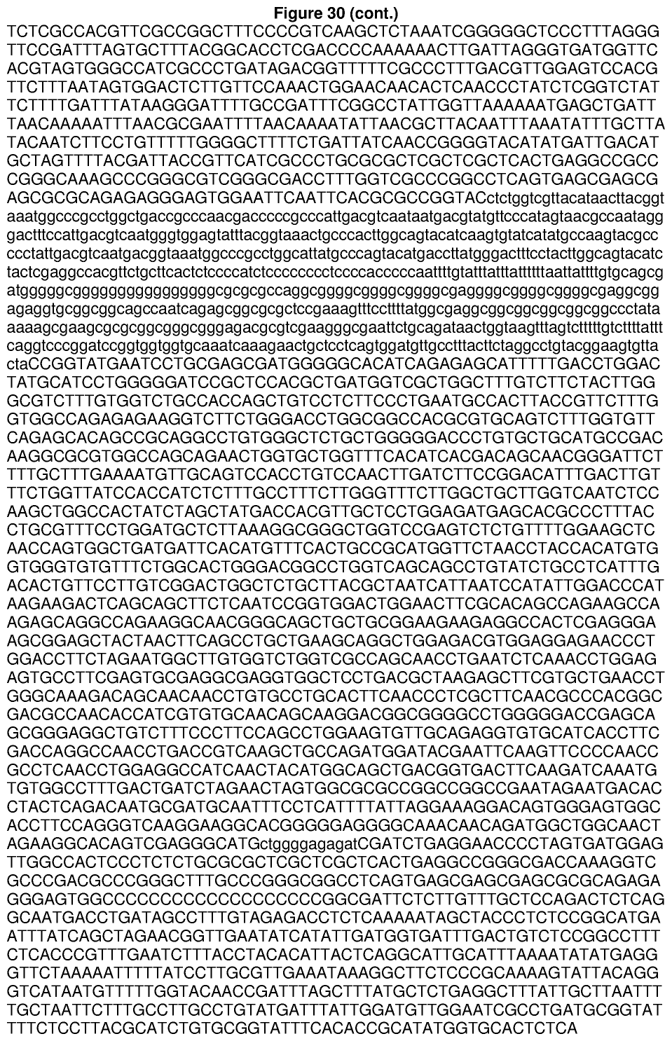

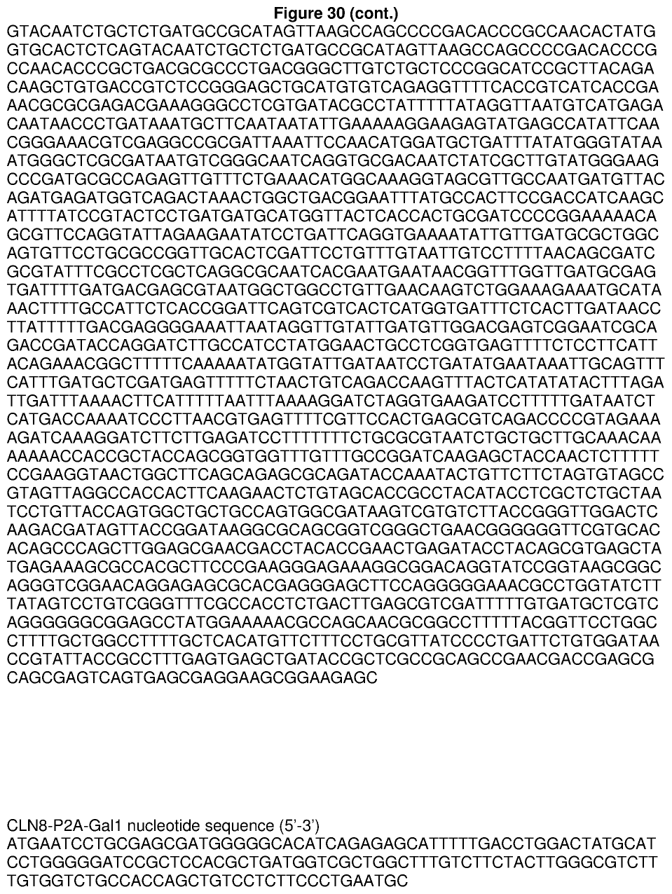

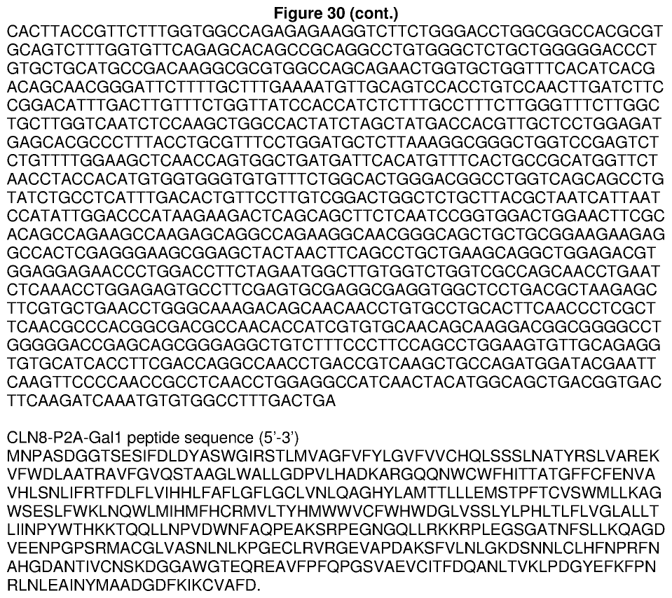

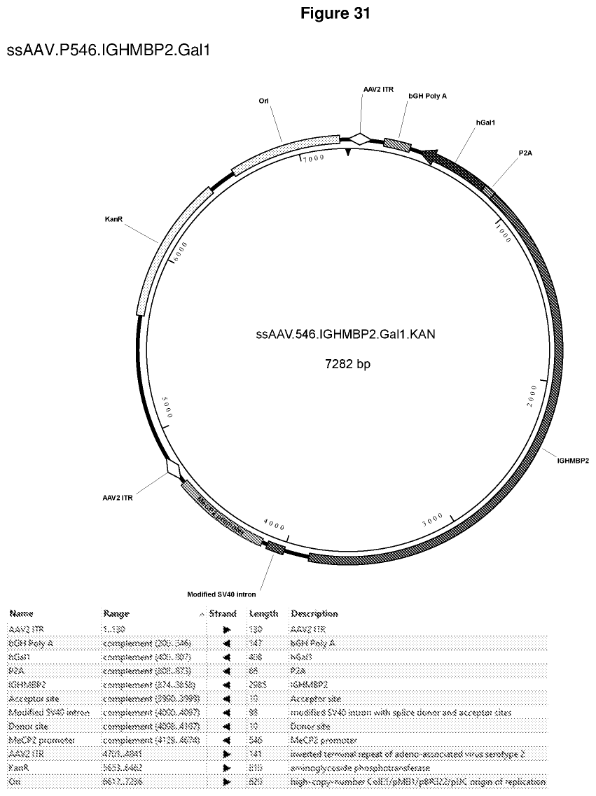

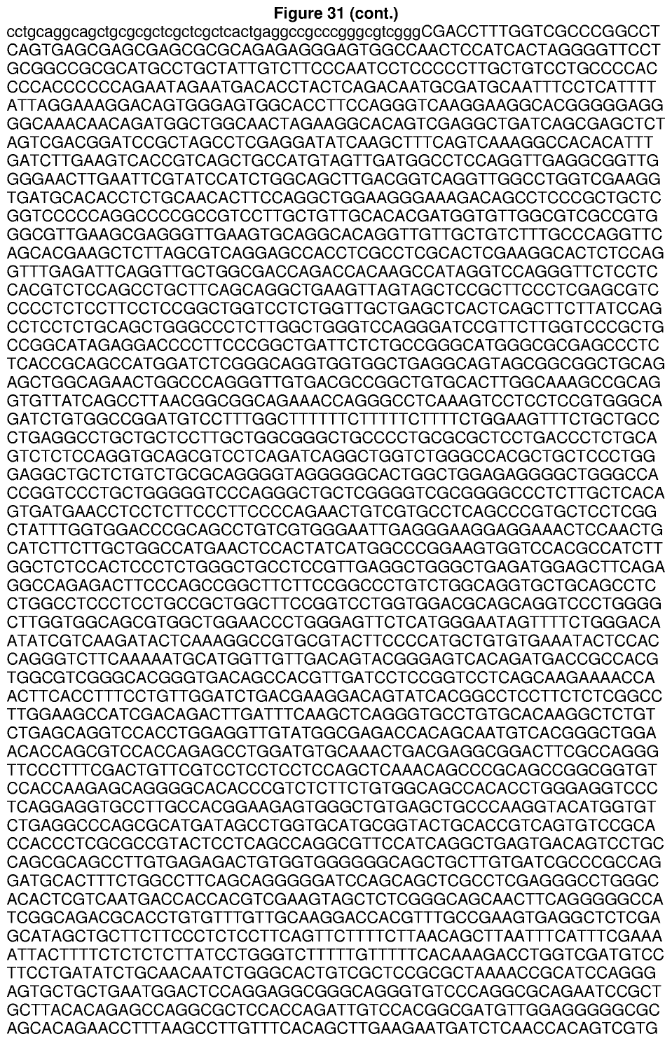

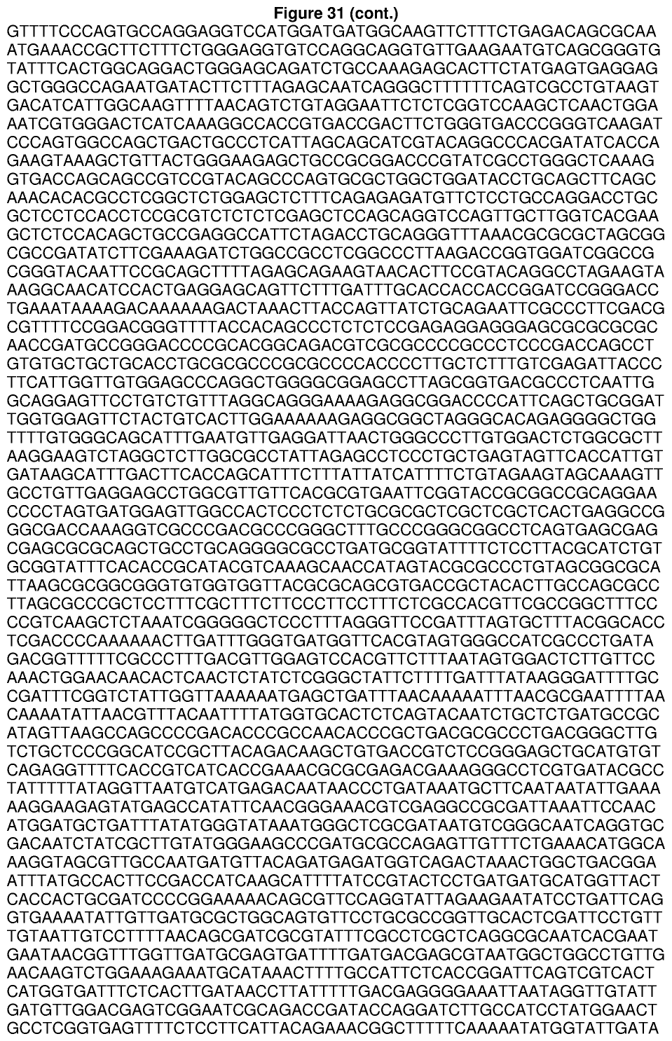

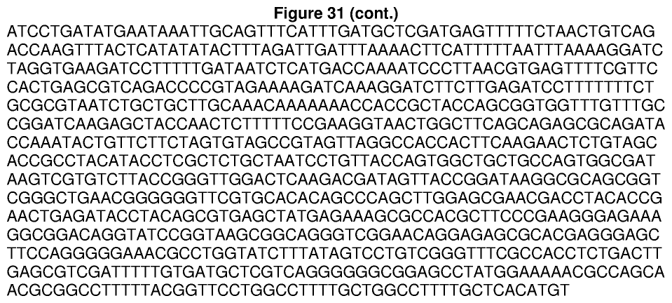

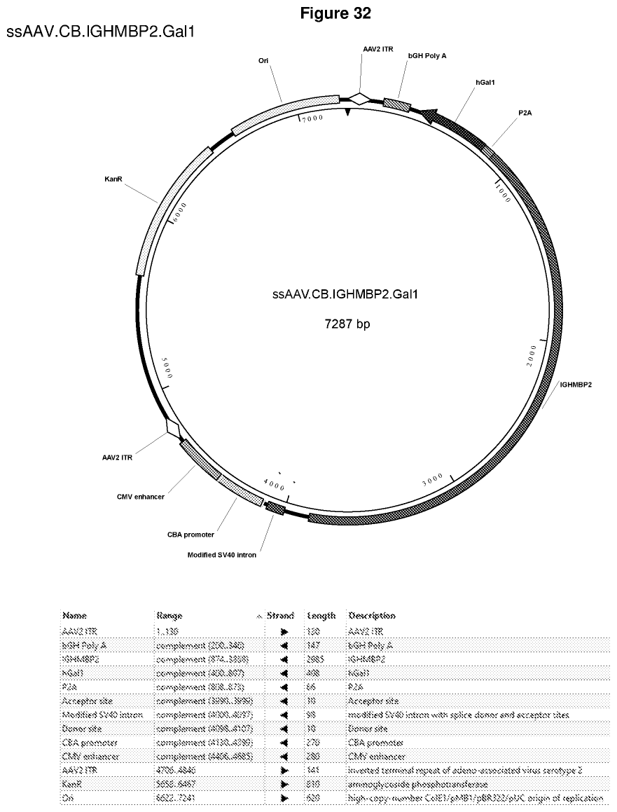

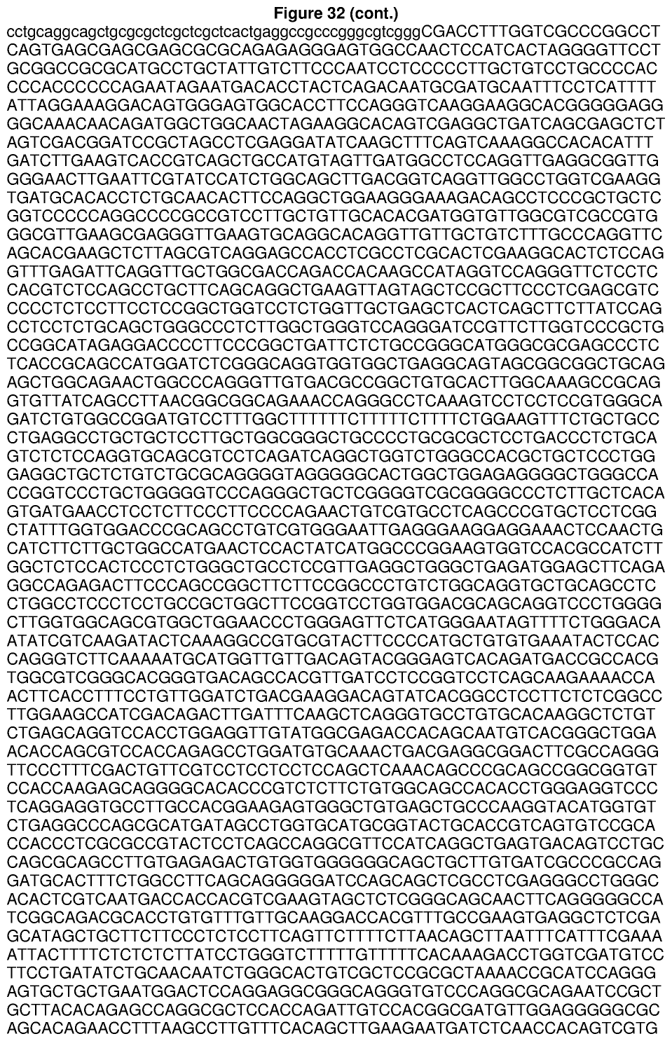

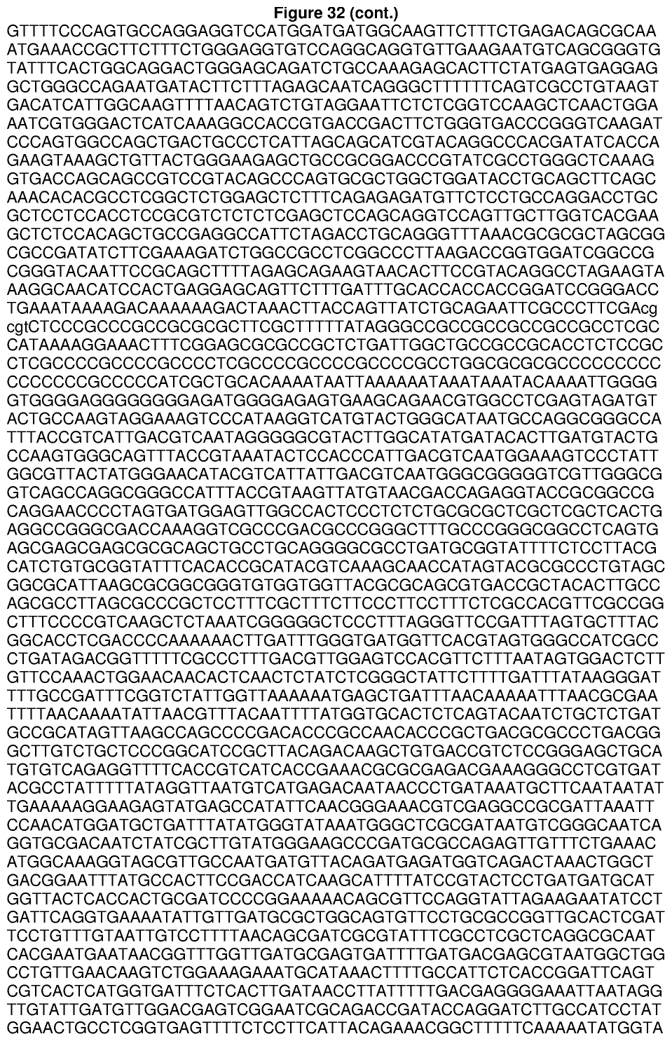

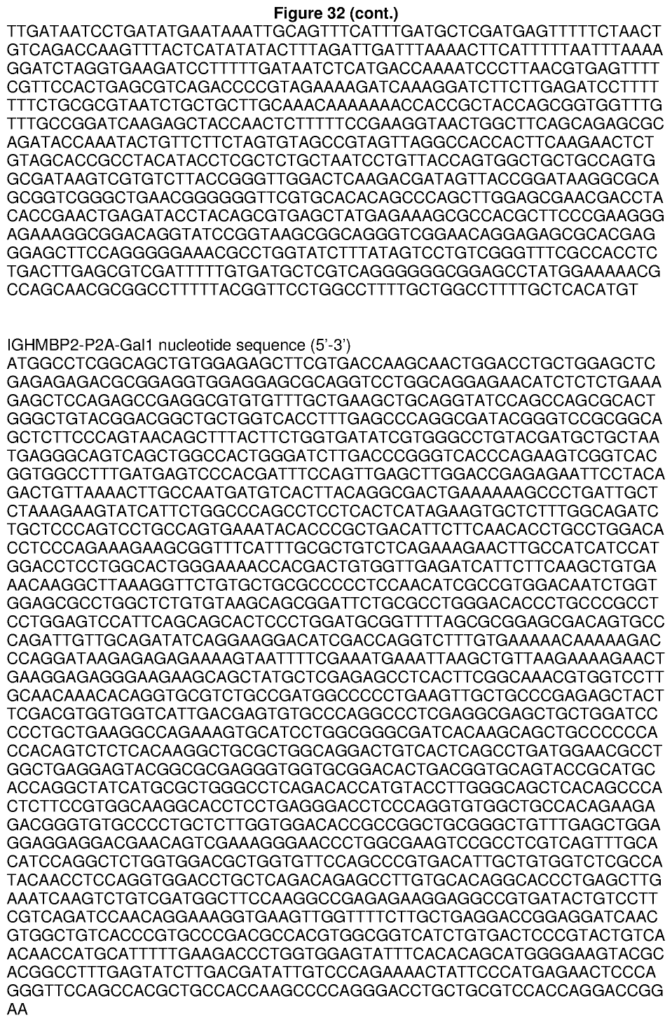

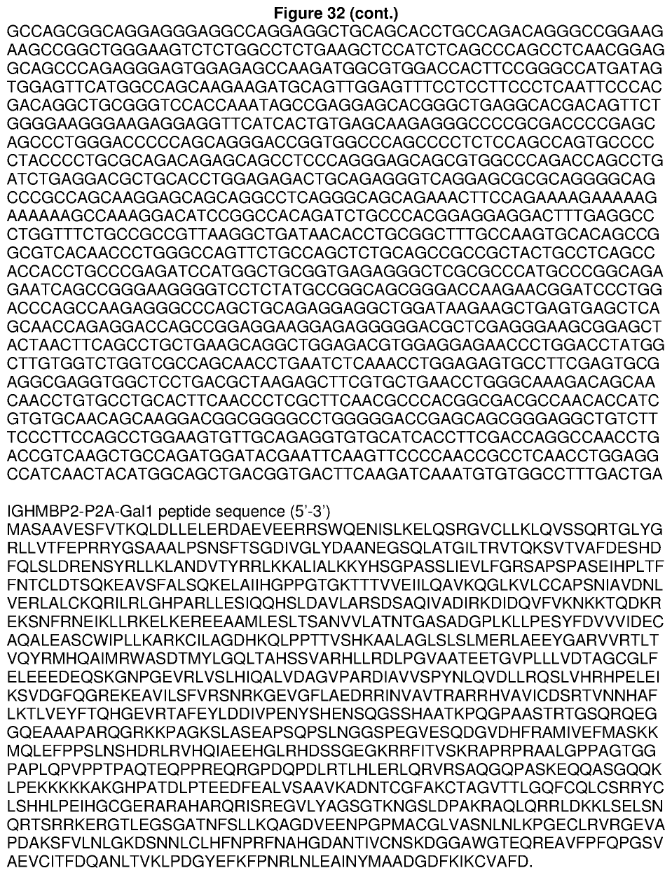

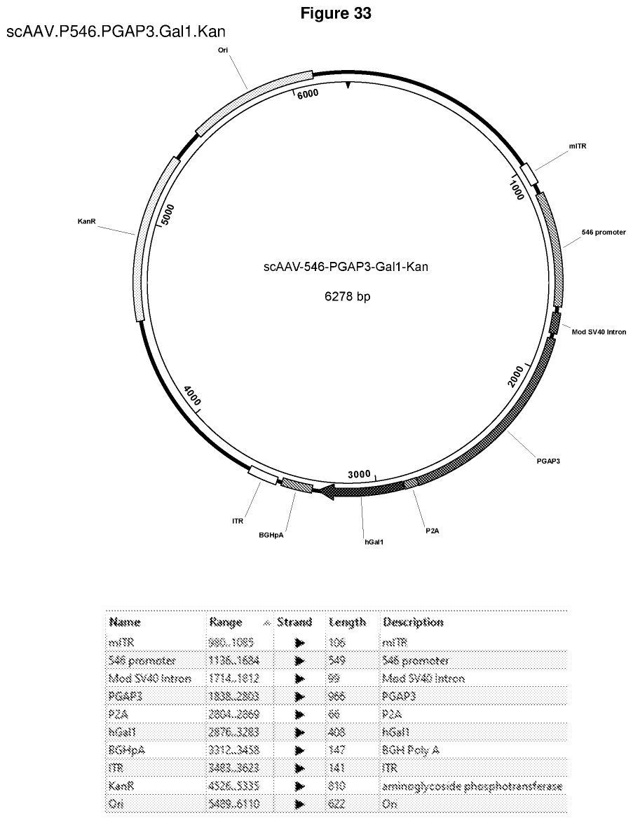

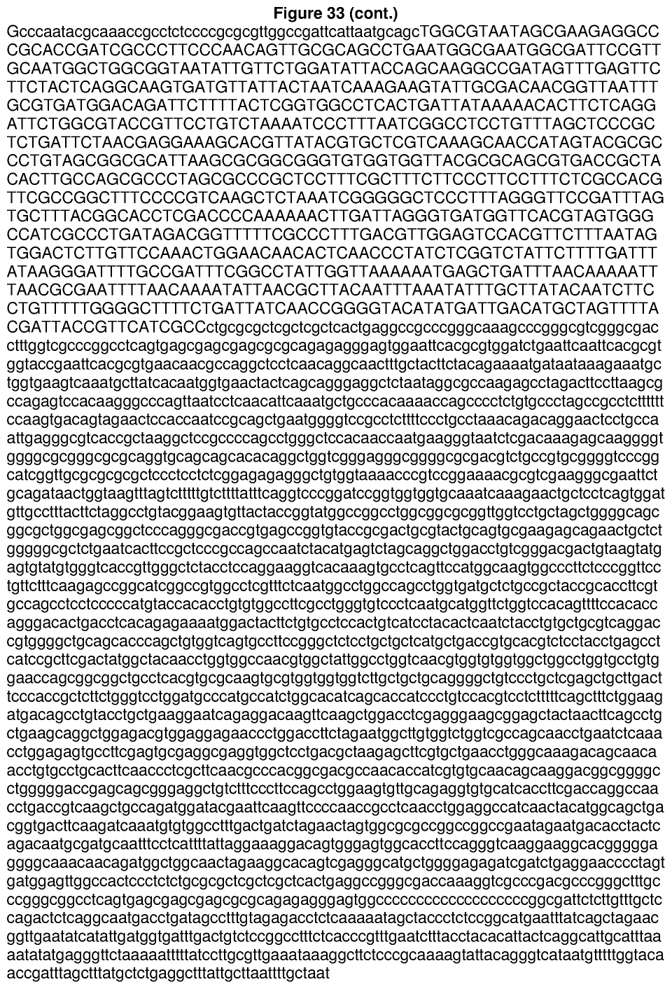

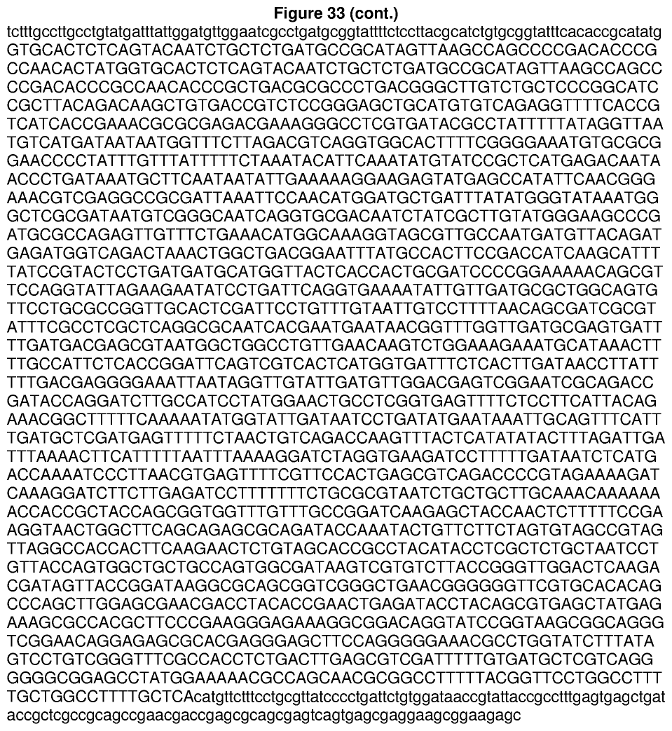

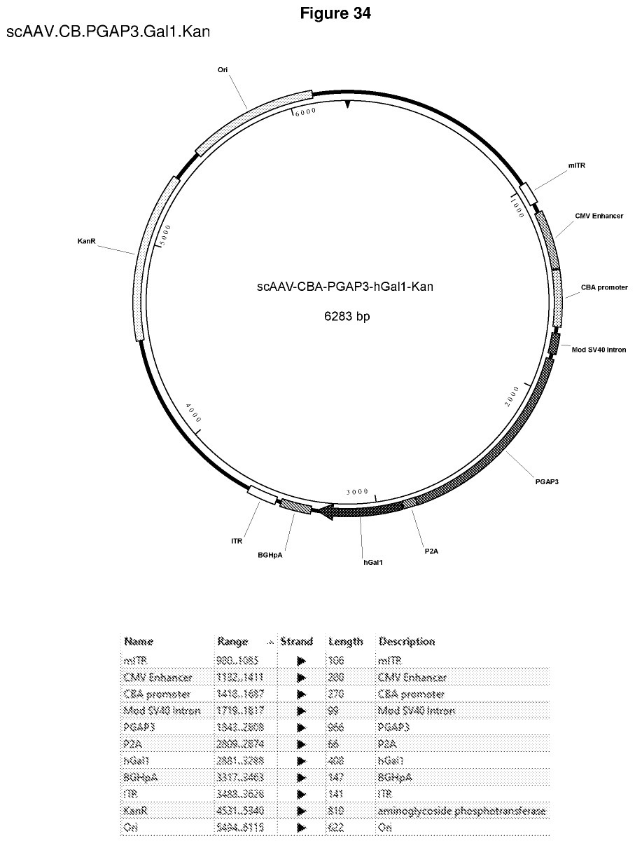

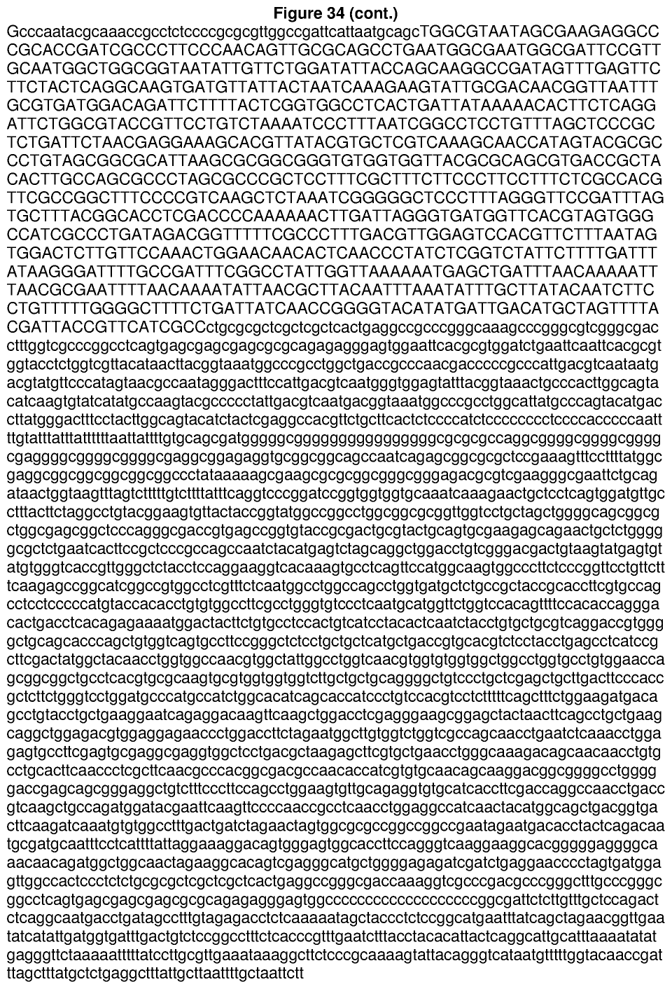

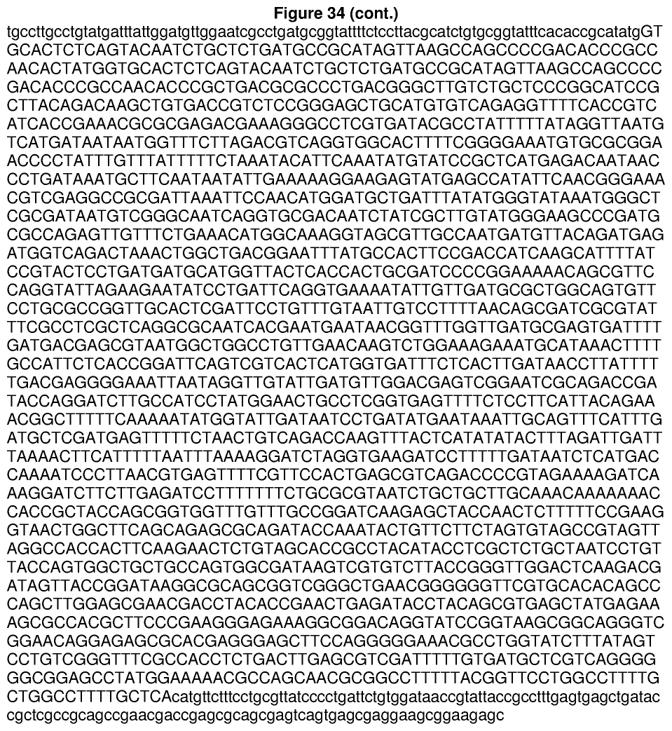

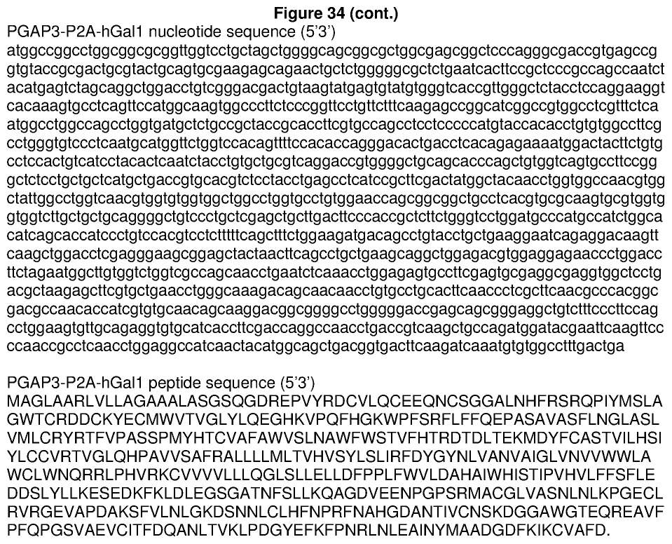

Various Galectin-1 fusion constructs (AAV production plasmids and sequences shown in FIGS. 23-34) were made depending on the underlying neurological/neurodegenerative disorder to be treated including, for example, Batten diseases (CLN1/3/6/8), IGHMBP2-related disorders, and PGAP3 Congenital Disorder of Glycosylation.

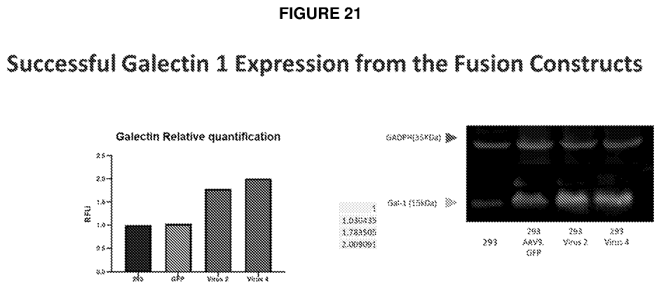

For example, AAV vectors expressing Galectin-1 as a fusion protein with PGAP3 gene of interest and P2A self-cleaving peptide were generated. The vectors were first tested in HEK293 cells to determine the faithful expression of Galectin-1. Western blot analysis revealed 1.78- to 2-fold expression of Galectin-1 from the fusion constructs as compared to untreated cells (FIG. 21).

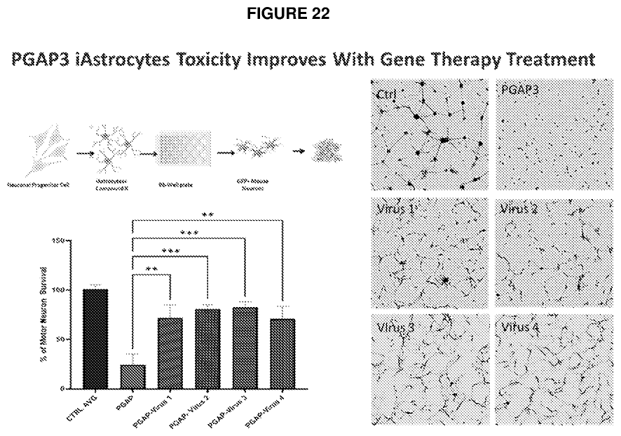

The vectors were further tested in a coculture assay where PGAP3 patient derived iAstrocytes treated with combination vectors showed significant improvement in neuronal survival (FIG. 22).

### Example 5

Human MTs are encoded by a family of ten genes (on chromosome 16) subdivided into four groups, MT-1 to MT-4. The genes/MTs are MT-1A, MT-1B, MT-1E, MT-1F, MT-1G, MT-1H, MT-1X, MT-2A, MT-3 and MT-4. MTs are small molecular weight proteins, with 6-7 kDa, composed of a single polypeptide chain containing 60 to 68 amino acid residues. Importantly, within the central nervous system, MT-1/2 and MT-3 are primarily expressed in astrocytes as compared to neurons. Expression of MTs is induced by metals, glucocorticoids, cytokines and variety of physical stress condition as well as ROS and oxidative stress, the hallmarks of multiple neurological and neurodegenerative disorders. The physiological function of MTs is considered to be metal cellular homeostasis and heavy metal detoxification. The present disclosure contemplates that MT-1/2 also play an important role in neuroprotection and neuro-regeneration. Owing to their zinc binding and antioxidant properties, MT-1/2 and MT-3 have also been implicated in promoting neuronal outgrowth, neuronal survival and reduction of apoptosis and brain inflammation. Thus, MTs are contemplated herein as therapeutic agents for multiple neurological and neurodegenerative disorders.

To achieve the correction of multiple cell types associated in neurological/neurodegenerative disorder, the present disclosure contemplates providing the intrinsic/extrinsic effects of neuro-immune modulatory proteins (like Galectin and NBD) with that of the metallothionines using rAAV to co-express them in the CNS. The disclosure provides illustrative plasmids/constructs (FIGS. 35-38) for producing rAAV for the treatment of ALS where a SOD1 shRNA expression cassette is combined with a Galectin1-P2A-MT1E expression cassette in the same AAV vector. Depending on the disease/disorder to be treated, the Galectin1-P2A-MT expression cassette can be combined with other gene of interest expression cassettes as well as multiple MTs that can be co-expressed using multiple 2A cleavage sequences.

We claim:

1. A recombinant adeno-associated virus (rAAV) genome that expresses (A) a therapeutic protein or RNA and (B) an anti-inflammatory protein or peptide.

2. The rAAV genome of claim wherein:
(A) is a short hairpin ribonucleic acid targeting superoxide dismutase 1 (SOD1 shRNA), or (A) is a CLN1, CLN3, CLN6, CLN8, IGHMBP2 or PGAP3 protein.

3. The rAAV genome of claim 2 wherein the sequence of the SOD1 shRNA is SEQ ID NO: 4.

4. The rAAV genome of any preceding claim wherein (B) is human Galectin-1 or human Galectin-3.

5. The rAAV genome of any of claim 1-3 wherein (B) is a metallothionein protein, a metallothionein fusion protein, NBD 1X or NBD 3X.

6. The rAAV genome of any preceding claim wherein the expression of (A) is under the control of an H1 promoter and the expression of (B) is under the control of a CBA promoter.

7. A rAAV comprising the genome of any preceding claim.

8. The rAAV of claim 7 that is a scAAV or a ssAAV.

9. The rAAV of claim 7 or 8 that comprises AAV9 capsid.

10. A rAAV wherein the rAAV is AAV.sh129SOD1.hGal1, AAV.hGalactin1, scAAV.P546.CLN1.Gal1, scAAV.CB. CLN1.Gal1, scAAV.P546.CLN3.Gal1, scAAV.CB.CLN3.Gal1, scAAV.CB.CLN6.Gal1, scAAV.P546.CLN8.Gal1, SCAAV.CB.CLN8.Gal1, scAAV.P546.IGHMBP2.Gal1, scAAV.CB.IGHMBP2.Gal1, SCAAV.546.PGAP3.Gal1 or scAAV.CBA.PGAP3.Gal1.

11. A composition comprising the rAAV of any of claims 7-10.

12. The composition of claim 11 further comprising an agent that increases the viscosity or density of the composition.

13. The composition of claim 12 wherein the agent is a contrast agent.

14. The composition of any one of claims 11-13, wherein the composition is formulated for direct injection into the cerebrospinal fluid, intracerebroventricular delivery, intrathecal delivery or intravenous delivery.

15. A method of treating Amyotrophic Lateral Sclerosis in a subject comprising administering to the subject an effective amount of the rAAV composition expressing a short hairpin ribonucleic acid targeting superoxide dismutase 1 (SOD1 shRNA) of any of claims 11-14.

16. The method of claim 15, wherein the rAAV composition is administered to the subject by direct injection into the cerebrospinal fluid, intracerebroventricular delivery, intrathecal delivery or intravenous delivery.

17. Use of the rAAV expressing a short hairpin ribonucleic acid targeting superoxide dismutase 1 (SOD1 shRNA) of any one of claims 7-10 in the preparation of a medicament for the treatment of ALS.

18. A plasmid comprising the rAAV genome of any of claims 1-6.

19. A method of producing a rAAV comprising the step of transducing a packaging cell with the plasmid of claim 18 and culturing the packaging cell.

20. A method of treating Batten disease, IGHMBP2-related disorder (SMARD1/CMT2S) or PGAP3 Congenital Disorder of Glycosylation in a subject comprising administering to the subject an effective amount of an rAAV composition of claim 2 expressing: for Batten disease a CLN1, CLN3, CLN6 or CLN8 protein, for IGHMBP2-related disorder a IGHMBP2 protein, or for PGAP3 Congenital Disorder of Glycosylation a PGAP3 protein.


SEQUENCE LISTING (81 sequences)

SEQ ID NO: 1 - RNA, 26 nt, synthetic construct
  source 1..26: mol_type = other RNA
gcatcatcaatttcgagcagaaggaa

SEQ ID NO: 2 - RNA, 23 nt, synthetic construct
  source 1..23: mol_type = other RNA
gaagcattaaaggactgactgaa

SEQ ID NO: 3 - RNA, 23 nt, synthetic construct
  source 1..23: mol_type = other RNA
ctgactgaaggcctgcatggatt

SEQ ID NO: 4 - RNA, 20 nt, synthetic construct
  source 1..20: mol_type = other RNA
catggattccatgttcatga

SEQ ID NO: 5 - RNA, 21 nt, synthetic construct
  source 1..21: mol_type = other RNA
gcatggattccatgttcatga

SEQ ID NO: 6 - RNA, 21 nt, synthetic construct
  source 1..21: mol_type = other RNA
ggtctggcctataaagtagtc

SEQ ID NO: 7 - RNA, 21 nt, synthetic construct
  source 1..21: mol_type = other RNA
gggcatcatcaatttcgagca

SEQ ID NO: 8 - RNA, 21 nt, synthetic construct
  source 1..21: mol_type = other RNA
gcatcatcaatttcgagcaga

SEQ ID NO: 9 - RNA, 21 nt, synthetic construct
  source 1..21: mol_type = other RNA
gcctgcatggattccatgttc

SEQ ID NO: 10 - RNA, 21 nt, synthetic construct
  source 1..21: mol_type = other RNA
ggaggtctggcctataaagta

SEQ ID NO: 11 - RNA, 21 nt, synthetic construct
  source 1..21: mol_type = other RNA
gattccatgttcatgagtttg

SEQ ID NO: 12 - RNA, 21 nt, synthetic construct
  source 1..21: mol_type = other RNA
ggagataatacagcaggctgt

SEQ ID NO: 13 - RNA, 21 nt, synthetic construct
  source 1..21: mol_type = other RNA
gctttaaagtacctgtagtga

SEQ ID NO: 14 - RNA, 21 nt, synthetic construct
  source 1..21: mol_type = other RNA
gcattaaaggactgactgaag

SEQ ID NO: 15 - RNA, 19 nt, synthetic construct
  source 1..19: mol_type = other RNA
tcatcaatttcgagcagaa

SEQ ID NO: 16 - RNA, 19 nt, synthetic construct
  source 1..19: mol_type = other RNA
tcgagcagaaggaaagtaa

SEQ ID NO: 17 - RNA, 19 nt, synthetic construct
  source 1..19: mol_type = other RNA
gcctgcatggattccatgt

SEQ ID NO: 18 - RNA, 19 nt, synthetic construct
  source 1..19: mol_type = other RNA
tcactctcaggagaccatt

SEQ ID NO: 19 - RNA, 19 nt, synthetic construct
  source 1..19: mol_type = other RNA
gctttaaagtacctgtagt

SEQ ID NO: 20 - DNA, 408 bp, Homo sapiens
  source 1..408: mol_type = genomic DNA
atggcttgtggtctggtcgccagcaacctgaatctcaaacctggagagtgccttcgagtgcgaggcgaggtggctcctgacgctaagagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgtgcatcaccttcgaccaggccaacctgaccgtcaagctgccagatggatacgaattcaagttccccaaccgcctcaacctggaggccatcaactacatggcagctgacggtgacttcaagatcaaatgtgtggcctttgactga

SEQ ID NO: 21 - DNA, 404 bp, Mus musculus
  source 1..404: mol_type = genomic DNA
atggcctgtggtctggtcgccagcaacctgaatctcaaacctggggaatgtctcaaagttcggggagaggtggcctcggacgccaagagctttgtgctgaacctgggaaaagacagcaacaacctgtgcctacacttcaatcctcgcttcaatgcccatggagacgccaacaccattgtgtgtaacaccaaggaagatgggacctggggaaccgaacaccgggaacctgccttccccttccagcccgggagcatcacagaggtgtgcatcacctttgaccaggctgacctgaccatcaagctgccagacggacatgaattcaagttccccaaccgcctcaacatggaggccatcaactacatggcggcggatggagacttcaagattaagtgcgtggcctttga

SEQ ID NO: 22 - DNA, 753 bp, Homo sapiens
  source 1..753: mol_type = genomic DNA
atggcagacaatttttcgctccatgatgcgttatctgggtctggaaacccaaaccctcaaggatggcctggcgcatgggggaaccagcctgctggggcagggggctacccaggggcttcctatcctggggcctaccccgggcaggcacccccaggggcttatcctggacaggcacctccaggcgcctaccctggagcacctggagcttatcccggagcacctgcacctggagtctacccagggccacccagcggccctggggcctacccatcttctggacagccaagtgccaccggagcctaccctgccactggcccctatggcgcccctgctgggccactgattgtgccttataacctgcctttgcctgggggagtggtgcctcgcatgctgataacaattctgggcacggtgaagcccaatgcaaacagaattgctttagatttccaaagagggaatgatgttgccttccactttaacccacgcttcaatgagaacaacaggagagtcattgtttgcaatacaaagctggataataactggggaagggaagaaagacagtcggttttcccatttgaaagtgggaaaccattcaaaatacaagtactggttgaacctgaccacttcaaggttgcagtgaatgatgctcacttgttgcagtacaatcatcgggttaaaaaactcaatgaaatcagcaaactgggaatttctggtgacatagacctcaccagtgcttcatataccatgatataa

SEQ ID NO: 23 - DNA, 795 bp, Mus musculus
  source 1..795: mol_type = genomic DNA
atggcagacagcttttcgcttaacgatgccttagctggctctggaaacccaaaccctcaaggatatccgggtgcatgggggaaccagcctggggcagggggctacccaggggctgcttatcctggggcctacccaggacaagctcctccaggggcctacccaggacaggctcctccaggggcctacccaggacaggctcctcctagtgcctaccccggcccaactgcccctggagcttatcctggcccaactgcccctggagcttatcctggctcaactgcccctggagccttcccagggcaacctggggcacctggggcctaccccagtgctcctggaggctatcctgctgctggcccttatggtgtccccgctggaccactgacggtgccctatgacctgcccttgcctggaggagtcatgccccgcatgctgatcacaatcatgggcacagtgaaacccaacgcaaacaggattgttctagatttcaggagagggaatgatgttgccttccactttaacccccgcttcaatgagaacaacaggagagtcattgtgtgtaacacgaagcaggacaataactggggaaaggaagaaagacagtcagccttcccctttgagagtggcaaaccattcaaaatacaagtcctggttgaagctgaccacttcaaggttgcggtcaacgatgctcacctactgcagtacaaccatcggatgaagaacctccgggaaatcagccaactggggatcagtggtgacataaccctcaccagcgctaaccacgccatgatctaa

SEQ ID NO: 24 - DNA, 333 bp, synthetic construct
  source 1..333: mol_type = other DNA
ccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgcgggatc

SEQ ID NO: 25 - DNA, 99 bp, synthetic construct
  source 1..99: mol_type = other DNA
aattcatatttgcatgtcgctatgtgttctgggaaatcaccataaacgtgaaatgtctttggatttgggaatcttataagttctgtatgagaccactcg

SEQ ID NO: 26 - DNA, 556 bp, synthetic construct
  source 1..556: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggag

SEQ ID NO: 27 - DNA, 546 bp, synthetic construct
  source 1..546: mol_type = other DNA
ttttccggacgggttttaccacagccctctctccgagaggagggagcgcgcgcgcaaccgatgccgggaccccgcacggcagacgtcgcgccccgccctcccgaccagcctgtgtgctgctgcacctgcgcgcccgcgccccaccccttgctctttgtcgagattacccttcattggttgtggagcccaggctggggcggagccttagcggtgacgccctcaattggcaggagttcctgtctgtttaggcagggaaaagaggcggaccccattcagctgcggattggtggagttctactgtcacttggaaaaaagaggcggctagggcacagaggggctggttttgtgggcagcatttgaatgttgaggattaactgggcccttgtggactctggcgcttaaggaagtctaggctcttggcgcctattagagcctccctgctgagtagttcaccattgtgataagcatttgacttcaccagcatttctttattatcattttctgtagaagtagcaaagttgcctgttgaggagcctggcgttgttc

SEQ ID NO: 28 - DNA, 485 bp, synthetic construct
  source 1..485: mol_type = other DNA
gtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaa

SEQ ID NO: 29 - DNA, 350 bp, synthetic construct
  source 1..350: mol_type = other DNA
gcatgtgtgggagtgaaattatggaatgtgtatgctcatagcactgagtgaaaataaaagattgtataaatcgtggggcttgtggaattgtgagtccctgtgcgtgtgcagtatttttttttttttttaagtaagactctttagatcttgtcgcctcccctgtcttctgtgattgattttgcgagactaatggtgcgtaaaagggctggtgagatctgggggcgcctcctagcctgacgtcagagagagagtttaaaaccgagggagacggttgagagcacacaagccgctttaggagtcgcgaggttcggagccatcgctgctgcctgctgatccgcgcctagagtttg

SEQ ID NO: 30 - DNA, 641 bp, synthetic construct
  source 1..641: mol_type = other DNA
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagcca

SEQ ID NO: 31 - DNA, 2214 bp, synthetic construct
  source 1..2214: mol_type = other DNA
acgcgtcccacctccctctctgtgctgggactcacagagggagacctcaggaggcagtctgtccatcacatgtccaaatgcagagcataccctgggctgggcgcagtggcgcacaactgtaattccagcactttgggaggctgatgtggaaggatcacttgagcccagaagttctagaccagcctgggcaacatggcaagaccctatctctacaaaaaaagttaaaaaatcagccacgtgtggtgacacacacctgtagtcccagctattcaggaggctgaggtgaggggatcacttaaggctgggaggttgaggctgcagtgagtcgtggttgcgccactgcactccagcctgggcaacagtgagaccctgtctcaaaagacaaaaaaaaaaaaaaaaaaaaaaagaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgtgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttccctttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgccgg

SEQ ID NO: 32 - DNA, 714 bp, synthetic construct
  source 1..714: mol_type = other DNA
ccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtgganccactacgggtctangctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggaccactacgggtctaggctgcccatgtaaggaggcaagcctggggacacccgagatgcctggttataattaaccccaacacctgctgccccccccccccaacacctgctgcctgagcctgagcggttaccccaccccggtgcctgggtcttaggctctgtacaccatggaggagaagctcgctctaaaaataaccctgtccctggtcctccctggggacagcccctcctggctagtcacaccctgtaggctcctctatataacccaggggcacaggggctgcccccgggtcact

SEQ ID NO: 33 - DNA, 781 bp, synthetic construct
  source 1..781: mol_type = other DNA
catgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagc

SEQ ID NO: 34 - RNA, 164 nt, synthetic construct
  source 1..164: mol_type = other RNA
aattcatatttgcatgtcgctatgtgttctgggaaatcaccataaacgtgaaatgtctttggatttgggaatcttataagttctgtatgagaccactcggatccatggattccatgttcatgattcaagagatcatgaacatggaatccatgcttttttggaaa

SEQ ID NO: 35 - DNA, 90 bp, synthetic construct
  source 1..90: mol_type = other DNA
atggacagacagatcaagatctggttccagaacagaagaatgaagtggaagaagaccgccctggactggagctggctgcagaccgagtaa

SEQ ID NO: 36 - protein, 29 aa, synthetic construct
MDRQIKIWFQNRRMKWKKTALDWSWLQTE

SEQ ID NO: 37 - DNA, 390 bp, synthetic construct
  source 1..390: mol_type = other DNA
atggacagacagatcaagatctggttccagaacagaagaatgaagtggaagaagaccgccctggactggagctggctgcagaccgagggaagcggagctactaacttcagcctgctgaagcaggctggagacgtggaggagaaccctggacctgacagacagatcaagatctggttccagaacagaagaatgaagtggaagaagaccgccctggactggagctggctgcagaccgagggaagcggagctactaacttcagcctgctgaagcaggctggagacgtggaggagaaccctggacctgacagacagatcaagatctggttccagaacagaagaatgaagtggaagaagaccgccctggactggagctggctgcagaccgagtaa

SEQ ID NO: 38 - protein, 129 aa, synthetic construct
MDRQIKIWFQNRRMKWKKTALDWSWLQTEGSGATNFSLLKQAGDVEENPGPDRQIKIWFQNRRMKWKKTALDWSWLQTEGSGATNFSLLKQAGDVEENPGPDRQIKIWFQNRRMKWKKTALDWSWLQTE

SEQ ID NO: 39 - protein, 21 aa, synthetic construct
GSGEGRGSLLTCGDVEENPGP

SEQ ID NO: 40 - protein, 22 aa, synthetic construct
GSGATNFSLLKQAGDVEENPGP

SEQ ID NO: 41 - protein, 23 aa, synthetic construct
GSGQCTNYALLKLAGDVESNPGP

SEQ ID NO: 42 - protein, 25 aa, synthetic construct
GSGVKQTLNFDLLKLAGDVESNPGP

SEQ ID NO: 43 - DNA, 5525 bp, synthetic construct
  source 1..5525: mol_type = other DNA
  unsure 610..715: note = Mutated ITR
  unsure 762..1041: note = CMV enhancer
  unsure 1048..1317: note = Chicken B-Actin promoter
  unsure 1383..1442: note = modSV40_late_16s_int
  unsure 1628..2035: note = msGalectin1
  unsure 2083..2229: note = BGHpA
  unsure 2309..2449: note = ITR
  unsure 3352..4212: note = Amp R
  unsure 4367..4986: note = Ori
ctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggttttaagggccgaggcggccagatctttcgaagatatcggcgccgctagcgcggccgcgtttaaaccctgcaggtctagaatcatggcctgtggtctggtcgccagcaacctgaatctcaaacctggggaatgtctcaaagttcggggagaggtggcctcggacgccaagagctttgtgctgaacctgggaaaagacagcaacaacctgtgcctacacttcaatcctcgcttcaatgcccatggagacgccaacaccattgtgtgtaacaccaaggaagatgggacctggggaaccgaacaccgggaacctgccttccccttccagcccgggagcatcacagaggtgtgcatcacctttgaccaggctgacctgaccatcaagctgccagacggacatgaattcaagttccccaaccgcctcaacatggaggccatcaactacatggcggcggatggagacttcaagattaagtgcgtggcctttgagtgaagctctagaaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgct

SEQ ID NO: 44 - DNA, 5935 bp, synthetic construct
  source 1..5935: mol_type = other DNA
  unsure 610..715: note = Mutated ITR
  unsure complement(849..913): note = SOD shRNA
  unsure complement(914..1012): note = H1
  unsure 1172..1451: note = CMV enhancer
  unsure 1458..1727: note = Chicken B-Actin promoter
  unsure 1793..1852: note = modSV40_late_16s_int
  unsure 2038..2441: note = msGalectin1
  unsure 2493..2639: note = BGHpA
  unsure 2719..2859: note = ITR
  unsure 3762..4622: note = Amp R
  unsure 4777..5396: note = Ori
ctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctacactttatgcttccggctcgtatgttgtgtggaattgtgagcggataacaatttcacacaggaaacagctatgaccatgattacgccaagctttccaaaaaagcatggattccatgttcatgatctcttgaatcatgaacatggaatccatggatccgagtggtctcatacagaacttataagattcccaaatccaaagacatttcacgtttatggtgatttcccagaacacatagcgacatgcaaatatgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggttttaagggccgaggcggccagatctttcgaagatatcggcgccgctagcgcggccgcgtttaaaccctgcaggtctagaatcatggcctgtggtctggtcgccagcaacctgaatctcaaacctggggaatgtctcaaagttcggggagaggtggcctcggacgccaagagctttgtgctgaacctgggaaaagacagcaacaacctgtgcctacacttcaatcctcgcttcaatgcccatggagacgccaacaccattgtgtgtaacaccaaggaagatgggacctggggaaccgaacaccgggaacctgccttccccttccagcccgggagcatcacagaggtgtgcatcacctttgaccaggctgacctgaccatcaagctgccagacggacatgaattcaagttccccaaccgcctcaacatggaggccatcaactacatggcggcggatggagacttcaagattaagtgcgtggcctttgagtgaagctctagaaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgct

SEQ ID NO: 45 - DNA, 5935 bp, synthetic construct
  source 1..5935: mol_type = other DNA
  unsure 610..715: note = Mutated ITR
  unsure complement(849..913): note = SOD shRNA
  unsure complement(914..1012): note = H1
  unsure 1172..1451: note = CMV enhancer
  unsure 1458..1727: note = Chicken B-Actin promoter
  unsure 1793..1889: note = modSV40_late_16s_int
  unsure 2036..2443: note = hGalectin1
  unsure 2493..2639: note = BGHpA
  unsure 2719..2859: note = ITR
  unsure 3762..4622: note = Amp R
  unsure 4777..5396: note = Ori
ctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctacactttatgcttccggctcgtatgttgtgtggaattgtgagcggataacaatttcacacaggaaacagctatgaccatgattacgccaagctttccaaaaaagcatggattccatgttcatgatctcttgaatcatgaacatggaatccatggatccgagtggtctcatacagaacttataagattcccaaatccaaagacatttcacgtttatggtgatttcccagaacacatagcgacatgcaaatatgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggttttaagggccgaggcggccagatctttcgaagatatcggcgccgctagcgcggccgcgtttaaaccctgcaggttctagaatggcttgtggtctggtcgccagcaacctgaatctcaaacctggagagtgccttcgagtgcgaggcgaggtggctcctgacgctaagagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgtgcatcaccttcgaccaggccaacctgaccgtcaagctgccagatggatacgaattcaagttccccaaccgcctcaacctggaggccatcaactacatggcagctgacggtgacttcaagatcaaatgtgtggcctttgactgatctagactagaaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgct

SEQ ID NO: 46 - DNA, 5519 bp, synthetic construct
  source 1..5519: mol_type = other DNA
  unsure 610..715: note = Mutated ITR
  unsure 762..1041: note = CMV enhancer
  unsure 1048..1317: note = Chicken B-Actin promoter
  unsure 1383..1442: note = modSV40_late_16s_int
  unsure 1620..2032: note = hGalectin1
  unsure 2077..2223: note = BGHpA
  unsure 2303..2443: note = ITR
  unsure 3346..4206: note = Amp R
  unsure 4361..4980: note = Ori
ctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggttttaagggccgaggcggccagatctttcgaagatatcggcgccgctagcgcggccgcgtttaaaccctgcaggtctagaatggcttgtggtctggtcgccagcaacctgaatctcaaacctggagagtgccttcgagtgcgaggcgaggtggctcctgacgctaagagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgtgcatcaccttcgaccaggccaacctgaccgtcaagctgccagatggatacgaattcaagttccccaaccgcctcaacctggaggccatcaactacatggcagctgacggtgacttcaagatcaaatgtgtggcctttgactgatctagaaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgct

SEQ ID NO: 47 - DNA, 6115 bp, synthetic construct
  source 1..6115: mol_type = other DNA
  unsure 980..1085: note = mITR
  unsure 1121..1666: note = P546 promoter
  unsure 1697..1796: note = Mod SV40 Intron
  unsure 1822..2739: note = CLN1
  unsure 2746..2811: note = P2A
  unsure 2818..3225: note = hGal1
  unsure 3254..3400: note = bGH Poly A
  unsure 3425..3565: note = ITR
  unsure 4363..5172: note = Kan R
  unsure 5327..5946: note = Ori
gcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcaattcacgcgccggtaccgaattcacgcgtgaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaacgcgtcgaagggcgaattctgcagataactggtaagtttagtcttttttgtcttttatttcaggtcccggatccggtggtggtgcaaatcaaagaactgctcctcagtcgatgttgcctttacttctaggcctgtacggaagtgttactaccggtatggcgtcgcccggctgcctgtggctcttggctgtggctctcctgccatggacctgcgcttctcgggcgctgcagcatctggacccgccggcgccgctgccgttggtgatctggcatgggatgggagacagctgttgcaatcccttaagcatgggtgctattaaaaaaatggtggagaagaaaatacctggaatttacgtcttatctttagagattgggaagaccctgatggaggacgtggagaacagcttcttcttgaatgtcaattcccaagtaacaacagtgtgtcaggcacttgctaaggatcctaaattgcagcaaggctacaatgctatgggattctcccagggaggccaatttctgagggcagtggctcagagatgcccttcacctcccatgatcaatctgatctcggttgggggacaacatcaaggtgtttttggactccctcgatgcccaggagagagctctcacatctgtgacttcatccgaaaaacactgaatgctggggcgtactccaaagttgttcaggaacgcctcgtgcaagccgaatactggcatgaccccataaaggaggatgtgtatcgcaaccacagcatcttcttggcagatataaatcaggagcggggtatcaatgagtcctacaagaaaaacctgatggccctgaagaagtttgtgatggtgaaattcctcaatgattccattgtggaccctgtagattcggagtggtttggattttacagaagtggccaagccaaggaaaccattcccttacaggagacctccctgtacacacaggaccgcctggggctaaaggaaatggacaatgcaggacagctagtgtttctggctacagaaggggaccatcttcagttgtctgaagaatggttttatgcccacatcataccattccttggactcgagggaagcggagctactaacttcagcctgctgaagcaggctggagacgtggaggagaaccctggaccttctagaatggcttgtggtctggtcgccagcaacctgaatctcaaacctggagagtgccttcgagtgcgaggcgaggtggctcctgacgctaagagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgtgcatcaccttcgaccaggccaacctgaccgtcaagctgccagatggatacgaattcaagttccccaaccgcctcaacctggaggccatcaactacatggcagctgacggtgacttcaagatcaaatgtgtggcctttgactgatctagaactagtggcgcgccggccggccgaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgagggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 48 - DNA, 6209 bp, synthetic construct
  source 1..6209: mol_type = other DNA
  unsure 1048..1317: note = CBA promoter
  unsure 1382..1480: note = Mod SV40 Intron
  unsure 1546..2463: note = CLN1
  unsure 2470..2535: note = P2A
  unsure 2542..2949: note = hGal1
  unsure 2978..3124: note = bGH Poly A
  unsure 3149..3289: note = ITR
  unsure complement(4134..4943): note = Kan R
  unsure 5051..5670: note = Ori
ctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtatggcgtcgcccggctgcctgtggctcttggctgtggctctcctgccatggacctgcgcttctcgggcgctgcagcatctggacccgccggcgccgctgccgttggtgatctggcatgggatgggagacagctgttgcaatcccttaagcatgggtgctattaaaaaaatggtggagaagaaaatacctggaatttacgtcttatctttagagattgggaagaccctgatggaggacgtggagaacagcttcttcttgaatgtcaattcccaagtaacaacagtgtgtcaggcacttgctaaggatcctaaattgcagcaaggctacaatgctatgggattctcccagggaggccaatttctgagggcagtggctcagagatgcccttcacctcccatgatcaatctgatctcggttgggggacaacatcaaggtgtttttggactccctcgatgcccaggagagagctctcacatctgtgacttcatccgaaaaacactgaatgctggggcgtactccaaagttgttcaggaacgcctcgtgcaagccgaatactggcatgaccccataaaggaggatgtgtatcgcaaccacagcatcttcttggcagatataaatcaggagcggggtatcaatgagtcctacaagaaaaacctgatggccctgaagaagtttgtgatggtgaaattcctcaatgattccattgtggaccctgtagattcggagtggtttggattttacagaagtggccaagccaaggaaaccattcccttacaggagacctccctgtacacacaggaccgcctggggctaaaggaaatggacaatgcaggacagctagtgtttctggctacagaaggggaccatcttcagttgtctgaagaatggttttatgcccacatcataccattccttggactcgagggaagcggagctactaacttcagcctgctgaagcaggctggagacgtggaggagaaccctggaccttctagaatggcttgtggtctggtcgccagcaacctgaatctcaaacctggagagtgccttcgagtgcgaggcgaggtggctcctgacgctaagagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgtgcatcaccttcgaccaggccaacctgaccgtcaagctgccagatggatacgaattcaagttccccaaccgcctcaacctggaggccatcaactacatggcagctgacggtgacttcaagatcaaatgtgtggcctttgactgatctagaactagtggcgcgccggccggccgaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgagggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgct

SEQ ID NO: 49 - DNA, 1333 bp, synthetic construct
  source 1..1333: mol_type = other DNA
atggcgtcgcccggctgcctgtggctcttggctgtggctctcctgccatggacctgcgcttctcgggcgctgcagcatctggacccgccggcgccgctgccgttggtgatctggcatgggatgggagacagctgttgcaatcccttaagcatgggtgctattaaaaaaatggtggagaagaaaatacctggaatttacgtcttatctttagagattgggaagaccctgatggaggacgtggagaacagcttcttcttgaatgtcaattcccaagtaacaacagtgtgtcaggcacttgctaaggatcctaaattgcagcaaggctacaatgctatgggattctcccagggaggccaatttctgagggcagtggctcagagatgcccttcacctcccatgatcaatctgatctcggttgggggacaacatcaaggtgtttttggactccctcgatgcccaggagagagctctcacatctgtgacttcatccgaaaaacactgaatgctggggcgtactccaaagttgttcaggaacgcctcgtgcaagccgaatactggcatgaccccataaaggaggatgtgtatcgcaaccacagcatcttcttggcagatataaatcaggagcggggtatcaatgagtcctacaagaaaaacctgatggccctgaagaagtttgtgatggtgaaattcctcaatgattccattgtggaccctgtagattcggagtggtttggattttacagaagtggccaagccaaggaaaccattcccttacaggagacctccctgtacacacaggaccgcctggggctaaaggaaatggacaatgcaggacagctagtgtttctggctacagaaggggaccatcttcagttgtctgaagaatggttttatgcccacatcataccattccttggactcgagggaagcggagctactaacttcagcctgctgaagcaggctggagacgtggaggagaaccctggaccttctagaatggcttgtggtctggtcgccagcaacctgaatctcaaacctggagagtgccttcgagtgcgaggcgaggtggctcctgacgctaagagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgtgcatcaccttcgaccaggccaacctgaccgtcaagctgccagatggatacgaattcaagttccccaaccgcc

SEQ ID NO: 50 - protein, 467 aa, synthetic construct
MASPGCLWLLAVALLPWTCASRALQHLDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLGLEGSGATNFSLLKQAGDVEENPGPSRMACGLVASNLNLKPGECLRVRGEVAPDAKSFVLNLGKDSNNLCLHFNPRFNAHGDANTIVCNSKDGGAWGTEQREAVFPFQPGSVAEVCITFDQANLTVKLPDGYEFKFPNRLNLEAINYMAADGDFKIKCVAFD

SEQ ID NO: 51 - DNA, 6511 bp, synthetic construct
  source 1..6511: mol_type = other DNA
  unsure 980..1085: note = mITR
  unsure 1121..1666: note = P546 promoter
  unsure 1697..1796: note = Mod SV40 Intron
  unsure 1822..3135: note = hCLN3 CDS
  unsure 3142..3207: note = P2A
  unsure 3214..3621: note = hGal1
  unsure 3650..3796: note = bGH Poly A
  unsure 3821..3961: note = ITR
  unsure 4759..5568: note = Kan R
  unsure 5723..6342: note = Ori
gcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcaattcacgcgccggtaccgaattcacgcgtgaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaacgcgtcgaagggcgaattctgcagataactggtaagtttagtcttttttgtcttttatttcaggtcccggatccggtggtggtgcaaatcaaagaactgctcctcagtcgatgttgcctttacttctaggcctgtacggaagtgttactaccggtatgggaggctgtgcaggctcgcggcggcgcttttcggattccgagggggaggagaccgtcccggagccccggctccctctgttggaccatcagggcgcgcattggaagaacgcggtgggcttctggctgctgggcctttgcaacaacttctcttatgtggtgatgctgagtgccgcccacgacatccttagccacaagaggacatcgggaaaccagagccatgtggacccaggcccaacgccgatcccccacaacagctcatcacgatttgactgcaactctgtctctacggctgctgtgctcctggcggacatcctccccacactcgtcatcaaattgttggctcctcttggccttcacctgctgccctacagcccccgggttctcgtcagtgggatttgtgctgctggaagcttcgtcctggttgccttttctcattctgtggggaccagcctgtgtggtgtggtcttcgctagcatctcatcaggccttggggaggtcaccttcctctccctcactgccttctaccccagggccgtgatctcctggtggtcctcagggactgggggagctgggctgctgggggccctgtcctacctgggcctcacccaggccggcctctcccctcagcagaccctgctgtccatgctgggtatccctgccctgctgctggccagctatttcttgttgctcacatctcctgaggcccaggaccctggaggggaagaagaagcagagagcgcagcccggcagcccctcataagaaccgaggccccggagtcgaagccaggctccagctccagcctctcccttcgggaaaggtggacagtgttcaagggtctgctgtggtacattgttcccttggtcgtagtttactttgccgagtatttcattaaccagggactttttgaactcctctttttctggaacacttccctgagtcacgctcagcaataccgctggtaccagatgctgtaccaggctggcgtctttgcctcccgctcttctctccgctgctgtcgcatccgtttcacctgggccctggccctgctgcagtgcctcaacctggtgttcctgctggcagacgtgtggttcggctttctgccaagcatctacctcgtcttcctgatcattctgtatgaggggctcctgggaggcgcagcctacgtgaacaccttccacaacatcgccctggagaccagtgatgagcaccgggagtttgcaatggcggccacctgcatctctgacacactggggatctccctgtcggggctcctggctttgcctctgcatgacttcctctgccagctctccctcgagggaagcggagctactaacttcagcctgctgaagcaggctggagacgtggaggagaaccctggaccttctagaatggcttgtggtctggtcgccagcaacctgaatctcaaacctggagagtgccttcgagtgcgaggcgaggtggctcctgacgctaagagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgtgcatcaccttcgaccaggccaacctgaccgtcaagctgccagatggatacgaattcaagttccccaaccgcctcaacctggaggccatcaactacatggcagctgacggtgacttcaagatcaaatgtgtggcctttgactgatctagaactagtggcgcgccggccggccgaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgagggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 52 - DNA, 6605 bp, synthetic construct
  source 1..6605: mol_type = other DNA
  unsure 610..715: note = mutated ITR
  unsure 762..1041: note = CMV enhancer
  unsure 1048..1317: note = CBA promoter
  unsure 1382..1481: note = Mod SV40 Intron
  unsure 1546..2859: note = hCLN3 CDS
  unsure 2866..2931: note = P2A
  unsure 2938..3345: note = hGal1
  unsure 3374..3520: note = bGH Poly A
  unsure 3545..3685: note = ITR
  unsure complement(4530..5339): note = Kan R
  unsure 5447..6066: note = Ori
ctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtatgggaggctgtgcaggctcgcggcggcgcttttcggattccgagggggaggagaccgtcccggagccccggctccctctgttggaccatcagggcgcgcattggaagaacgcggtgggcttctggctgctgggcctttgcaacaacttctcttatgtggtgatgctgagtgccgcccacgacatccttagccacaagaggacatcgggaaaccagagccatgtggacccaggcccaacgccgatcccccacaacagctcatcacgatttgactgcaactctgtctctacggctgctgtgctcctggcggacatcctccccacactcgtcatcaaattgttggctcctcttggccttcacctgctgccctacagcccccgggttctcgtcagtgggatttgtgctgctggaagcttcgtcctggttgccttttctcattctgtggggaccagcctgtgtggtgtggtcttcgctagcatctcatcaggccttggggaggtcaccttcctctccctcactgccttctaccccagggccgtgatctcctggtggtcctcagggactgggggagctgggctgctgggggccctgtcctacctgggcctcacccaggccggcctctcccctcagcagaccctgctgtccatgctgggtatccctgccctgctgctggccagctatttcttgttgctcacatctcctgaggcccaggaccctggaggggaagaagaagcagagagcgcagcccggcagcccctcataagaaccgaggccccggagtcgaagccaggctccagctccagcctctcccttcgggaaaggtggacagtgttcaagggtctgctgtggtacattgttcccttggtcgtagtttactttgccgagtatttcattaaccagggactttttgaactcctctttttctggaacacttccctgagtcacgctcagcaataccgctggtaccagatgctgtaccaggctggcgtctttgcctcccgctcttctctccgctgctgtcgcatccgtttcacctgggccctggccctgctgcagtgcctcaacctggtgttcctgctggcagacgtgtggttcggctttctgccaagcatctacctcgtcttcctgatcattctgtatgaggggctcctgggaggcgcagcctacgtgaacaccttccacaacatcgccctggagaccagtgatgagcaccgggagtttgcaatggcggccacctgcatctctgacacactggggatctccctgtcggggctcctggctttgcctctgcatgacttcctctgccagctctccctcgagggaagcggagctactaacttcagcctgctgaagcaggctggagacgtggaggagaaccctggaccttctagaatggcttgtggtctggtcgccagcaacctgaatctcaaacctggagagtgccttcgagtgcgaggcgaggtggctcctgacgctaagagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgtgcatcaccttcgaccaggccaacctgaccgtcaagctgccagatggatacgaattcaagttccccaaccgcctcaacctggaggccatcaactacatggcagctgacggtgacttcaagatcaaatgtgtggcctttgactgatctagaactagtggcgcgccggccggccgaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgagggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgct

SEQ ID NO: 53 - DNA, 1800 bp, synthetic construct
  source 1..1800: mol_type = other DNA
atgggaggctgtgcaggctcgcggcggcgcttttcggattccgagggggaggagaccgtcccggagccccggctccctctgttggaccatcagggcgcgcattggaagaacgcggtgggcttctggctgctgggcctttgcaacaacttctcttatgtggtgatgctgagtgccgcccacgacatccttagccacaagaggacatcgggaaaccagagccatgtggacccaggcccaacgccgatcccccacaacagctcatcacgatttgactgcaactctgtctctacggctgctgtgctcctggcggacatcctccccacactcgtcatcaaattgttggctcctcttggccttcacctgctgccctacagcccccgggttctcgtcagtgggatttgtgctgctggaagcttcgtcctggttgccttttctcattctgtggggaccagcctgtgtggtgtggtcttcgctagcatctcatcaggccttggggaggtcaccttcctctccctcactgccttctaccccagggccgtgatctcctggtggtcctcagggactgggggagctgggctgctgggggccctgtcctacctgggcctcacccaggccggcctctcccctcagcagaccctgctgtccatgctgggtatccctgccctgctgctggccagctatttcttgttgctcacatctcctgaggcccaggaccctggaggggaagaagaagcagagagcgcagcccggcagcccctcataagaaccgaggccccggagtcgaagccaggctccagctccagcctctcccttcgggaaaggtggacagtgttcaagggtctgctgtggtacattgttcccttggtcgtagtttactttgccgagtatttcattaaccagggactttttgaactcctctttttctggaacacttccctgagtcacgctcagcaataccgctggtaccagatgctgtaccaggctggcgtctttgcctcccgctcttctctccgctgctgtcgcatccgtttcacctgggccctggccctgctgcagtgcctcaacctggtgttcctgctggcagacgtgtggttcggctttctgccaagcatctacctcgtcttcctgatcattctgtatgaggggctcctgggaggcgcagcctacgtgaacaccttccacaacatcgccctggagaccagtgatgagcaccgggagtttgcaatggcggccacctgcatctctgacacactggggatctccctgtcggggctcctggctttgcctctgcatgacttcctctgccagctctccctcgagggaagcggagctactaacttcagcctgctgaagcaggctggagacgtggaggagaaccctggaccttctagaatggcttgtggtctggtcgccagcaacctgaatctcaaacctggagagtgccttcgagtgcgaggcgaggtggctcctgacgctaagagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgtgcatcaccttcgaccaggccaacctgaccgtcaagctgccagatggatacgaattcaagttccccaaccgcctcaacctggaggccatcaactacatggcagctgacggtgacttcaagatcaaatgtgtggcctttgactga

SEQ ID NO: 54 - protein, 599 aa, synthetic construct
MGGCAGSRRRFSDSEGEETVPEPRLPLLDHQGAHWKNAVGFWLLGLCNNFSYVVMLSAAHDILSHKRTSGNQSHVDPGPTPIPHNSSSRFDCNSVSTAAVLLADILPTLVIKLLAPLGLHLLPYSPRVLVSGICAAGSFVLVAFSHSVGTSLCGVVFASISSGLGEVTFLSLTAFYPRAVISWWSSGTGGAGLLGALSYLGLTQAGLSPQQTLLSMLGIPALLLASYFLLLTSPEAQDPGGEEEAESAARQPLIRTEAPESKPGSSSSLSLRERWTVFKGLLWYIVPLVVVYFAEYFINQGLFELLFFWNTSLSHAQQYRWYQMLYQAGVFASRSSLRCCRIRFTWALALLQCLNLVFLLADVWFGFLPSIYLVFLIILYEGLLGGAAYVNTFHNIALETSDEHREFAMAATCISDTLGISLSGLLALPLHDFLCQLSLEGSGATNFSLLKQAGDVEENPGPSRMACGLVASNLNLKPGECLRVRGEVAPDAKSFVLNLGKDSNNLCLHFNPRFNAHGDANTIVCNSKDGGAWGTEQREAVFPFQPGSVAEVCITFDQANLTVKLPDGYEFKFPNRLNLEAINYMAADGDFKIKCVAFD

SEQ ID NO: 55 - DNA, 6437 bp, synthetic construct
  source 1..6437: mol_type = other DNA
  unsure 610..715: note = Mutated ITR
  unsure 762..1041: note = CMV enhancer
  unsure 1048..1317: note = CBA promoter
  unsure 1382..1480: note = Intron
  unsure 1603..2535: note = hCLN6 cDNA
  unsure 2542..2607: note = P2A
  unsure 2614..3021: note = hGal1
  unsure 3050..3196: note = bGH Poly A
  unsure 3221..3361: note = ITR
  unsure 4264..5124: note = Amp R
  unsure 5279..5898: note = Ori
ctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcttaagggccgaggcggccagatctttcgaagatatcggcgccgctagcgcggccgcatggaggcgacgcggaggcggcagcacctgggagcgacgggcggcccaggcgcgcagctgggcgcctccttcctgcaggccaggcatggctctgtgagcgctgatgaggctgcccgcacggctcccttccacctcgacctctggttctacttcacactgcagaactgggttctggactttgggcgtcccattgccatgctggtattccctctcgagtggtttccactcaacaagcccagtgttggggactacttccacatggcctacaacgtcatcacgccctttctcttgctcaagctcatcgagcggtccccccgcaccctgccacgctccatcacgtacgtgagcatcatcatcttcatcatgggtgccagcatccacctggtgggtgactctgtcaaccaccgcctgctcttcagtggctaccagcaccacctgtctgtccgtgagaaccccatcatcaagaatctcaagccggagacgctgatcgactcctttgagctgctctactattatgatgagtacctgggtcactgcatgtggtacatccccttcttcctcatcctcttcatgtacttcagcggctgctttactgcctctaaagctgagagcttgattccagggcctgccctgctcctggtggcacccagtggcctgtactactggtacctggtcaccgagggccagatcttcatcctcttcatcttcaccttcttcgccatgctggccctcgtcctgcaccagaagcgcaagcgcctcttcctggacagcaacggcctcttcctcttctcctccttcgcactgaccctcttgcttgtggcgctctgggtcgcctggctgtggaatgaccctgttctcaggaagaagtacccgggtgtcatctacgtccctgagccctgggctttctacacccttcacgtcagcagtcggcacctcgagggaagcggagctactaacttcagcctgctgaagcaggctggagacgtggaggagaaccctggaccttctagaatggcttgtggtctggtcgccagcaacctgaatctcaaacctggagagtgccttcgagtgcgaggcgaggtggctcctgacgctaagagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgtgcatcaccttcgaccaggccaacctgaccgtcaagctgccagatggatacgaattcaagttccccaaccgcctcaacctggaggccatcaactacatggcagctgacggtgacttcaagatcaaatgtgtggcctttgactgatctagaactagtggcgcgccggccggccgaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgagggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgct

SEQ ID NO: 56 - DNA, 6281 bp, synthetic construct
  source 1..6281: mol_type = other DNA
  unsure 610..715: note = mutated ITR
  unsure 762..1041: note = CMV enhancer
  unsure 1048..1317: note = CBA promoter
  unsure 1382..1480: note = Mod SV40 Intron
  unsure 1603..2535: note = hCLN6 CDS
  unsure 2542..2607: note = P2A
  unsure 2614..3021: note = hGal1
  unsure 3050..3196: note = bGH Poly A
  unsure 3221..3361: note = ITR
  unsure complement(4206..5015): note = Kan R
  unsure 5123..5742: note = Ori
ctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcttaagggccgaggcggccagatctttcgaagatatcggcgccgctagcgcggccgcatggaggcgacgcggaggcggcagcacctgggagcgacgggcggcccaggcgcgcagctgggcgcctccttcctgcaggccaggcatggctctgtgagcgctgatgaggctgcccgcacggctcccttccacctcgacctctggttctacttcacactgcagaactgggttctggactttgggcgtcccattgccatgctggtattccctctcgagtggtttccactcaacaagcccagtgttggggactacttccacatggcctacaacgtcatcacgccctttctcttgctcaagctcatcgagcggtccccccgcaccctgccacgctccatcacgtacgtgagcatcatcatcttcatcatgggtgccagcatccacctggtgggtgactctgtcaaccaccgcctgctcttcagtggctaccagcaccacctgtctgtccgtgagaaccccatcatcaagaatctcaagccggagacgctgatcgactcctttgagctgctctactattatgatgagtacctgggtcactgcatgtggtacatccccttcttcctcatcctcttcatgtacttcagcggctgctttactgcctctaaagctgagagcttgattccagggcctgccctgctcctggtggcacccagtggcctgtactactggtacctggtcaccgagggccagatcttcatcctcttcatcttcaccttcttcgccatgctggccctcgtcctgcaccagaagcgcaagcgcctcttcctggacagcaacggcctcttcctcttctcctccttcgcactgaccctcttgcttgtggcgctctgggtcgcctggctgtggaatgaccctgttctcaggaagaagtacccgggtgtcatctacgtccctgagccctgggctttctacacccttcacgtcagcagtcggcacctcgagggaagcggagctactaacttcagcctgctgaagcaggctggagacgtggaggagaaccctggaccttctagaatggcttgtggtctggtcgccagcaacctgaatctcaaacctggagagtgccttcgagtgcgaggcgaggtggctcctgacgctaagagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgtgcatcaccttcgaccaggccaacctgaccgtcaagctgccagatggatacgaattcaagttccccaaccgcctcaacctggaggccatcaactacatggcagctgacggtgacttcaagatcaaatgtgtggcctttgactgatctagaactagtggcgcgccggccggccgaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgagggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgct

SEQ ID NO: 57 - DNA, 1419 bp, synthetic construct
  source 1..1419: mol_type = other DNA
atggaggcgacgcggaggcggcagcacctgggagcgacgggcggcccaggcgcgcagctgggcgcctccttcctgcaggccaggcatggctctgtgagcgctgatgaggctgcccgcacggctcccttccacctcgacctctggttctacttcacactgcagaactgggttctggactttgggcgtcccattgccatgctggtattccctctcgagtggtttccactcaacaagcccagtgttggggactacttccacatggcctacaacgtcatcacgccctttctcttgctcaagctcatcgagcggtccccccgcaccctgccacgctccatcacgtacgtgagcatcatcatcttcatcatgggtgccagcatccacctggtgggtgactctgtcaaccaccgcctgctcttcagtggctaccagcaccacctgtctgtccgtgagaaccccatcatcaagaatctcaagccggagacgctgatcgactcctttgagctgctctactattatgatgagtacctgggtcactgcatgtggtacatccccttcttcctcatcctcttcatgtacttcagcggctgctttactgcctctaaagctgagagcttgattccagggcctgccctgctcctggtggcacccagtggcctgtactactggtacctggtcaccgagggccagatcttcatcctcttcatcttcaccttcttcgccatgctggccctcgtcctgcaccagaagcgcaagcgcctcttcctggacagcaacggcctcttcctcttctcctccttcgcactgaccctcttgcttgtggcgctctgggtcgcctggctgtggaatgaccctgttctcaggaagaagtacccgggtgtcatctacgtccctgagccctgggctttctacacccttcacgtcagcagtcggcacctcgagggaagcggagctactaacttcagcctgctgaagcaggctggagacgtggaggagaaccctggaccttctagaatggcttgtggtctggtcgccagcaacctgaatctcaaacctggagagtgccttcgagtgcgaggcgaggtggctcctgacgctaagagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgtgcatcaccttcgaccaggccaacctgaccgtcaagctgccagatggatacgaattcaagttccccaaccgcctcaacctggaggccatcaactacatggcagctgacggtgacttcaagatcaaatgtgtggcctttgactga

SEQ ID NO: 58 - protein, 472 aa, synthetic construct
MEATRRRQHLGATGGPGAQLGASFLQARHGSVSADEAARTAPFHLDLWFYFTLQNWVLDFGRPIAMLVFPLEWFPLNKPSVGDYFHMAYNVITPFLLLKLIERSPRTLPRSITYVSIIIFIMGASIHLVGDSVNHRLLFSGYQHHLSVRENPIIKNLKPETLIDSFELLYYYDEYLGHCMWYIPFFLILFMYFSGCFTASKAESLIPGPALLLVAPSGLYYWYLVTEGQIFILFIFTFFAMLALVLHQKRKRLFLDSNGLFLFSSFALTLLLVALWVAWLWNDPVLRKKYPGVIYVPEPWAFYTLHVSSRHLEGSGATNFSLLKQAGDVEENPGPSRMACGLVASNLNLKPGECLRVRGEVAPDAKSFVLNLGKDSNNLCLHFNPRFNAHGDANTIVCNSKDGGAWGTEQREAVFPFQPGSVAEVCITFDQANLTVKLPDGYEFKFPNRLNLEAINYMAADGDFKIKCVAFD

SEQ ID NO: 59 - DNA, 6055 bp, synthetic construct
  source 1..6055: mol_type = other DNA
  unsure 980..1085: note = mutated ITR
  unsure 1121..1666: note = P546 Promoter
  unsure 1697..1796: note = Mod SV40 Intron
  unsure 1822..2679: note = hCLN8
  unsure 2686..2751: note = P2A
  unsure 2758..3165: note = hGal1
  unsure 3194..3340: note = bGH Poly A
  unsure 3365..3505: note = ITR
  unsure 4303..5112: note = KanR
  unsure 5267..5886: note = Ori
gcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcaattcacgcgccggtaccgaattcacgcgtgaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaacgcgtcgaagggcgaattctgcagataactggtaagtttagtcttttttgtcttttatttcaggtcccggatccggtggtggtgcaaatcaaagaactgctcctcagtcgatgttgcctttacttctaggcctgtacggaagtgttactaccggtatgaatcctgcgagcgatgggggcacatcagagagcatttttgacctggactatgcatcctgggggatccgctccacgctgatggtcgctggctttgtcttctacttgggcgtctttgtggtctgccaccagctgtcctcttccctgaatgccacttaccgttctttggtggccagagagaaggtcttctgggacctggcggccacgcgtgcagtctttggtgttcagagcacagccgcaggcctgtgggctctgctgggggaccctgtgctgcatgccgacaaggcgcgtggccagcagaactggtgctggtttcacatcacgacagcaacgggattcttttgctttgaaaatgttgcagtccacctgtccaacttgatcttccggacatttgacttgtttctggttatccaccatctctttgcctttcttgggtttcttggctgcttggtcaatctccaagctggccactatctagctatgaccacgttgctcctggagatgagcacgccctttacctgcgtttcctggatgctcttaaaggcgggctggtccgagtctctgttttggaagctcaaccagtggctgatgattcacatgtttcactgccgcatggttctaacctaccacatgtggtgggtgtgtttctggcactgggacggcctggtcagcagcctgtatctgcctcatttgacactgttccttgtcggactggctctgcttacgctaatcattaatccatattggacccataagaagactcagcagcttctcaatccggtggactggaacttcgcacagccagaagccaagagcaggccagaaggcaacgggcagctgctgcggaagaagaggccactcgagggaagcggagctactaacttcagcctgctgaagcaggctggagacgtggaggagaaccctggaccttctagaatggcttgtggtctggtcgccagcaacctgaatctcaaacctggagagtgccttcgagtgcgaggcgaggtggctcctgacgctaagagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgtgcatcaccttcgaccaggccaacctgaccgtcaagctgccagatggatacgaattcaagttccccaaccgcctcaacctggaggccatcaactacatggcagctgacggtgacttcaagatcaaatgtgtggcctttgactgatctagaactagtggcgcgccggccggccgaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgagggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 60 - DNA, 6059 bp, synthetic construct
  source 1..6059: mol_type = other DNA
  unsure 980..1085: note = Mutated ITR
  unsure 1115..1394: note = CMV Enhancer
  unsure 1401..1670: note = CBA promoter
  unsure 1702..1800: note = Mod SV40 Intron
  unsure 1826..2683: note = hCLN8
  unsure 2690..2755: note = P2A
  unsure 2762..3169: note = hGal1
  unsure 3198..3344: note = bGH Poly A
  unsure 3369..3509: note = ITR
  unsure 4307..5116: note = Kan R
  unsure 5271..5890: note = Ori
gcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcaattcacgcgccggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagacgcgtcgaagggcgaattctgcagataactggtaagtttagtctttttgtcttttatttcaggtcccggatccggtggtggtgcaaatcaaagaactgctcctcagtggatgttgcctttacttctaggcctgtacggaagtgttactaccggtatgaatcctgcgagcgatgggggcacatcagagagcatttttgacctggactatgcatcctgggggatccgctccacgctgatggtcgctggctttgtcttctacttgggcgtctttgtggtctgccaccagctgtcctcttccctgaatgccacttaccgttctttggtggccagagagaaggtcttctgggacctggcggccacgcgtgcagtctttggtgttcagagcacagccgcaggcctgtgggctctgctgggggaccctgtgctgcatgccgacaaggcgcgtggccagcagaactggtgctggtttcacatcacgacagcaacgggattcttttgctttgaaaatgttgcagtccacctgtccaacttgatcttccggacatttgacttgtttctggttatccaccatctctttgcctttcttgggtttcttggctgcttggtcaatctccaagctggccactatctagctatgaccacgttgctcctggagatgagcacgccctttacctgcgtttcctggatgctcttaaaggcgggctggtccgagtctctgttttggaagctcaaccagtggctgatgattcacatgtttcactgccgcatggttctaacctaccacatgtggtgggtgtgtttctggcactgggacggcctggtcagcagcctgtatctgcctcatttgacactgttccttgtcggactggctctgcttacgctaatcattaatccatattggacccataagaagactcagcagcttctcaatccggtggactggaacttcgcacagccagaagccaagagcaggccagaaggcaacgggcagctgctgcggaagaagaggccactcgagggaagcggagctactaacttcagcctgctgaagcaggctggagacgtggaggagaaccctggaccttctagaatggcttgtggtctggtcgccagcaacctgaatctcaaacctggagagtgccttcgagtgcgaggcgaggtggctcctgacgctaagagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgtgcatcaccttcgaccaggccaacctgaccgtcaagctgccagatggatacgaattcaagttccccaaccgcctcaacctggaggccatcaactacatggcagctgacggtgacttcaagatcaaatgtgtggcctttgactgatctagaactagtggcgcgccggccggccgaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgagggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 61 - DNA, 1344 bp, synthetic construct
  source 1..1344: mol_type = other DNA
atgaatcctgcgagcgatgggggcacatcagagagcatttttgacctggactatgcatcctgggggatccgctccacgctgatggtcgctggctttgtcttctacttgggcgtctttgtggtctgccaccagctgtcctcttccctgaatgccacttaccgttctttggtggccagagagaaggtcttctgggacctggcggccacgcgtgcagtctttggtgttcagagcacagccgcaggcctgtgggctctgctgggggaccctgtgctgcatgccgacaaggcgcgtggccagcagaactggtgctggtttcacatcacgacagcaacgggattcttttgctttgaaaatgttgcagtccacctgtccaacttgatcttccggacatttgacttgtttctggttatccaccatctctttgcctttcttgggtttcttggctgcttggtcaatctccaagctggccactatctagctatgaccacgttgctcctggagatgagcacgccctttacctgcgtttcctggatgctcttaaaggcgggctggtccgagtctctgttttggaagctcaaccagtggctgatgattcacatgtttcactgccgcatggttctaacctaccacatgtggtgggtgtgtttctggcactgggacggcctggtcagcagcctgtatctgcctcatttgacactgttccttgtcggactggctctgcttacgctaatcattaatccatattggacccataagaagactcagcagcttctcaatccggtggactggaacttcgcacagccagaagccaagagcaggccagaaggcaacgggcagctgctgcggaagaagaggccactcgagggaagcggagctactaacttcagcctgctgaagcaggctggagacgtggaggagaaccctggaccttctagaatggcttgtggtctggtcgccagcaacctgaatctcaaacctggagagtgccttcgagtgcgaggcgaggtggctcctgacgctaagagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgtgcatcaccttcgaccaggccaacctgaccgtcaagctgccagatggatacgaattcaagttccccaaccgcctcaacctggaggccatcaactacatggcagctgacggtgacttcaagatcaaatgtgtggcctttgactga

SEQ ID NO: 62 - protein, 447 aa, synthetic construct
MNPASDGGTSESIFDLDYASWGIRSTLMVAGFVFYLGVFVVCHQLSSSLNATYRSLVAREKVFWDLAATRAVFGVQSTAAGLWALLGDPVLHADKARGQQNWCWFHITTATGFFCFENVAVHLSNLIFRTFDLFLVIHHLFAFLGFLGCLVNLQAGHYLAMTTLLLEMSTPFTCVSWMLLKAGWSESLFWKLNQWLMIHMFHCRMVLTYHMWWVCFWHWDGLVSSLYLPHLTLFLVGLALLTLIINPYWTHKKTQQLLNPVDWNFAQPEAKSRPEGNGQLLRKKRPLEGSGATNFSLLKQAGDVEENPGPSRMACGLVASNLNLKPGECLRVRGEVAPDAKSFVLNLGKDSNNLCLHFNPRFNAHGDANTIVCNSKDGGAWGTEQREAVFPFQPGSVAEVCITFDQANLTVKLPDGYEFKFPNRLNLEAINYMAADGDFKIKCVAFD

SEQ ID NO: 63 - DNA, 7282 bp, synthetic construct
  source 1..7282: mol_type = other DNA
  unsure 1..130: note = AAV2 ITR
  unsure complement(200..346): note = bGH Poly A
  unsure complement(400..807): note = hGal1
  unsure complement(808..873): note = P2A
  unsure complement(874..3858): note = IGHMBP2
  unsure complement(3990..3999): note = Acceptor site
  unsure complement(4000..4097): note = Modified SV40 intron
  unsure complement(4098..4107): note = Donor site
  unsure complement(4129..4674): note = MeCP2 promoter
  unsure 4701..4841: note = AAV2 ITR
  unsure 5653..6462: note = KanR
  unsure 6617..7236: note = Ori
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagtcgacggatccgctagcctcgaggatatcaagctttcagtcaaaggccacacatttgatcttgaagtcaccgtcagctgccatgtagttgatggcctccaggttgaggcggttggggaacttgaattcgtatccatctggcagcttgacggtcaggttggcctggtcgaaggtgatgcacacctctgcaacacttccaggctggaagggaaagacagcctcccgctgctcggtcccccaggccccgccgtccttgctgttgcacacgatggtgttggcgtcgccgtgggcgttgaagcgagggttgaagtgcaggcacaggttgttgctgtctttgcccaggttcagcacgaagctcttagcgtcaggagccacctcgcctcgcactcgaaggcactctccaggtttgagattcaggttgctggcgaccagaccacaagccataggtccagggttctcctccacgtctccagcctgcttcagcaggctgaagttagtagctccgcttccctcgagcgtccccctctccttcctccggctggtcctctggttgctgagctcactcagcttcttatccagcctcctctgcagctgggccctcttggctgggtccagggatccgttcttggtcccgctgccggcatagaggaccccttcccggctgattctctgccgggcatgggcgcgagccctctcaccgcagccatggatctcgggcaggtggtggctgaggcagtagcggcggctgcagagctggcagaactggcccagggttgtgacgccggctgtgcacttggcaaagccgcaggtgttatcagccttaacggcggcagaaaccagggcctcaaagtcctcctccgtgggcagatctgtggccggatgtcctttggcttttttctttttcttttctggaagtttctgctgccctgaggcctgctgctccttgctggcgggctgcccctgcgcgctcctgaccctctgcagtctctccaggtgcagcgtcctcagatcaggctggtctgggccacgctgctccctgggaggctgctctgtctgcgcaggggtagggggcactggctggagaggggctgggccaccggtccctgctgggggtcccagggctgctcggggtcgcggggccctcttgctcacagtgatgaacctcctcttcccttccccagaactgtcgtgcctcagcccgtgctcctcggctatttggtggacccgcagcctgtcgtgggaattgagggaaggaggaaactccaactgcatcttcttgctggccatgaactccactatcatggcccggaagtggtccacgccatcttggctctccactccctctgggctgcctccgttgaggctgggctgagatggagcttcagaggccagagacttcccagccggcttcttccggccctgtctggcaggtgctgcagcctcctggcctccctcctgccgctggcttccggtcctggtggacgcagcaggtccctggggcttggtggcagcgtggctggaaccctgggagttctcatgggaatagttttctgggacaatatcgtcaagatactcaaaggccgtgcgtacttccccatgctgtgtgaaatactccaccagggtcttcaaaaatgcatggttgttgacagtacgggagtcacagatgaccgccacgtggcgtcgggcacgggtgacagccacgttgatcctccggtcctcagcaagaaaaccaacttcacctttcctgttggatctgacgaaggacagtatcacggcctccttctctcggccttggaagccatcgacagacttgatttcaagctcagggtgcctgtgcacaaggctctgtctgagcaggtccacctggaggttgtatggcgagaccacagcaatgtcacgggctggaacaccagcgtccaccagagcctggatgtgcaaactgacgaggcggacttcgccagggttccctttcgactgttcgtcctcctcctccagctcaaacagcccgcagccggcggtgtccaccaagagcaggggcacacccgtctcttctgtggcagccacacctgggaggtccctcaggaggtgccttgccacggaagagtgggctgtgagctgcccaaggtacatggtgtctgaggcccagcgcatgatagcctggtgcatgcggtactgcaccgtcagtgtccgcaccaccctcgcgccgtactcctcagccaggcgttccatcaggctgagtgacagtcctgccagcgcagccttgtgagagactgtggtggggggcagctgcttgtgatcgcccgccaggatgcactttctggccttcagcagggggatccagcagctcgcctcgagggcctgggcacactcgtcaatgaccaccacgtcgaagtagctctcgggcagcaacttcagggggccatcggcagacgcacctgtgtttgttgcaaggaccacgtttgccgaagtgaggctctcgagcatagctgcttcttccctctccttcagttcttttcttaacagcttaatttcatttcgaaaattacttttctctctcttatcctgggtctttttgtttttcacaaagacctggtcgatgtccttcctgatatctgcaacaatctgggcactgtcgctccgcgctaaaaccgcatccagggagtgctgctgaatggactccaggaggcgggcagggtgtcccaggcgcagaatccgctgcttacacagagccaggcgctccaccagattgtccacggcgatgttggagggggcgcagcacagaacctttaagccttgtttcacagcttgaagaatgatctcaaccacagtcgtggttttcccagtgccaggaggtccatggatgatggcaagttctttctgagacagcgcaaatgaaaccgcttctttctgggaggtgtccaggcaggtgttgaagaatgtcagcgggtgtatttcactggcaggactgggagcagatctgccaaagagcacttctatgagtgaggaggctgggccagaatgatacttctttagagcaatcagggcttttttcagtcgcctgtaagtgacatcattggcaagttttaacagtctgtaggaattctctcggtccaagctcaactggaaatcgtgggactcatcaaaggccaccgtgaccgacttctgggtgacccgggtcaagatcccagtggccagctgactgccctcattagcagcatcgtacaggcccacgatatcaccagaagtaaagctgttactgggaagagctgccgcggacccgtatcgcctgggctcaaaggtgaccagcagccgtccgtacagcccagtgcgctggctggatacctgcagcttcagcaaacacacgcctcggctctggagctctttcagagagatgttctcctgccaggacctgcgctcctccacctccgcgtctctctcgagctccagcaggtccagttgcttggtcacgaagctctccacagctgccgaggccattctagacctgcagggtttaaacgcgcgctagcggcgccgatatcttcgaaagatctggccgcctcggcccttaagaccggtggatcggccgcgggtacaattccgcagcttttagagcagaagtaacacttccgtacaggcctagaagtaaaggcaacatccactgaggagcagttctttgatttgcaccaccaccggatccgggacctgaaataaaagacaaaaaagactaaacttaccagttatctgcagaattcgcccttcgacgcgttttccggacgggttttaccacagccctctctccgagaggagggagcgcgcgcgcaaccgatgccgggaccccgcacggcagacgtcgcgccccgccctcccgaccagcctgtgtgctgctgcacctgcgcgcccgcgccccaccccttgctctttgtcgagattacccttcattggttgtggagcccaggctggggcggagccttagcggtgacgccctcaattggcaggagttcctgtctgtttaggcagggaaaagaggcggaccccattcagctgcggattggtggagttctactgtcacttggaaaaaagaggcggctagggcacagaggggctggttttgtgggcagcatttgaatgttgaggattaactgggcccttgtggactctggcgcttaaggaagtctaggctcttggcgcctattagagcctccctgctgagtagttcaccattgtgataagcatttgacttcaccagcatttctttattatcattttctgtagaagtagcaaagttgcctgttgaggagcctggcgttgttcacgcgtgaattcggtaccgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 64 - DNA, 7287 bp, synthetic construct
  source 1..7287: mol_type = other DNA
  unsure 1..130: note = AAV2 ITR
  unsure complement(200..346): note = bGH Poly A
  unsure complement(874..3858): note = IGHMBP2
  unsure complement(400..807): note = hGal1
  unsure complement(808..873): note = P2A
  unsure complement(3990..3999): note = Acceptor site
  unsure complement(4000..4097): note = Modified SV40 intron
  unsure complement(4098..4107): note = Donor site
  unsure complement(4130..4399): note = CBA promoter
  unsure complement(4406..4685): note = CMV enhancer
  unsure 4706..4846: note = AAV2 ITR
  unsure 5658..6467: note = KanR
  unsure 6622..7241: note = Ori
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagtcgacggatccgctagcctcgaggatatcaagctttcagtcaaaggccacacatttgatcttgaagtcaccgtcagctgccatgtagttgatggcctccaggttgaggcggttggggaacttgaattcgtatccatctggcagcttgacggtcaggttggcctggtcgaaggtgatgcacacctctgcaacacttccaggctggaagggaaagacagcctcccgctgctcggtcccccaggccccgccgtccttgctgttgcacacgatggtgttggcgtcgccgtgggcgttgaagcgagggttgaagtgcaggcacaggttgttgctgtctttgcccaggttcagcacgaagctcttagcgtcaggagccacctcgcctcgcactcgaaggcactctccaggtttgagattcaggttgctggcgaccagaccacaagccataggtccagggttctcctccacgtctccagcctgcttcagcaggctgaagttagtagctccgcttccctcgagcgtccccctctccttcctccggctggtcctctggttgctgagctcactcagcttcttatccagcctcctctgcagctgggccctcttggctgggtccagggatccgttcttggtcccgctgccggcatagaggaccccttcccggctgattctctgccgggcatgggcgcgagccctctcaccgcagccatggatctcgggcaggtggtggctgaggcagtagcggcggctgcagagctggcagaactggcccagggttgtgacgccggctgtgcacttggcaaagccgcaggtgttatcagccttaacggcggcagaaaccagggcctcaaagtcctcctccgtgggcagatctgtggccggatgtcctttggcttttttctttttcttttctggaagtttctgctgccctgaggcctgctgctccttgctggcgggctgcccctgcgcgctcctgaccctctgcagtctctccaggtgcagcgtcctcagatcaggctggtctgggccacgctgctccctgggaggctgctctgtctgcgcaggggtagggggcactggctggagaggggctgggccaccggtccctgctgggggtcccagggctgctcggggtcgcggggccctcttgctcacagtgatgaacctcctcttcccttccccagaactgtcgtgcctcagcccgtgctcctcggctatttggtggacccgcagcctgtcgtgggaattgagggaaggaggaaactccaactgcatcttcttgctggccatgaactccactatcatggcccggaagtggtccacgccatcttggctctccactccctctgggctgcctccgttgaggctgggctgagatggagcttcagaggccagagacttcccagccggcttcttccggccctgtctggcaggtgctgcagcctcctggcctccctcctgccgctggcttccggtcctggtggacgcagcaggtccctggggcttggtggcagcgtggctggaaccctgggagttctcatgggaatagttttctgggacaatatcgtcaagatactcaaaggccgtgcgtacttccccatgctgtgtgaaatactccaccagggtcttcaaaaatgcatggttgttgacagtacgggagtcacagatgaccgccacgtggcgtcgggcacgggtgacagccacgttgatcctccggtcctcagcaagaaaaccaacttcacctttcctgttggatctgacgaaggacagtatcacggcctccttctctcggccttggaagccatcgacagacttgatttcaagctcagggtgcctgtgcacaaggctctgtctgagcaggtccacctggaggttgtatggcgagaccacagcaatgtcacgggctggaacaccagcgtccaccagagcctggatgtgcaaactgacgaggcggacttcgccagggttccctttcgactgttcgtcctcctcctccagctcaaacagcccgcagccggcggtgtccaccaagagcaggggcacacccgtctcttctgtggcagccacacctgggaggtccctcaggaggtgccttgccacggaagagtgggctgtgagctgcccaaggtacatggtgtctgaggcccagcgcatgatagcctggtgcatgcggtactgcaccgtcagtgtccgcaccaccctcgcgccgtactcctcagccaggcgttccatcaggctgagtgacagtcctgccagcgcagccttgtgagagactgtggtggggggcagctgcttgtgatcgcccgccaggatgcactttctggccttcagcagggggatccagcagctcgcctcgagggcctgggcacactcgtcaatgaccaccacgtcgaagtagctctcgggcagcaacttcagggggccatcggcagacgcacctgtgtttgttgcaaggaccacgtttgccgaagtgaggctctcgagcatagctgcttcttccctctccttcagttcttttcttaacagcttaatttcatttcgaaaattacttttctctctcttatcctgggtctttttgtttttcacaaagacctggtcgatgtccttcctgatatctgcaacaatctgggcactgtcgctccgcgctaaaaccgcatccagggagtgctgctgaatggactccaggaggcgggcagggtgtcccaggcgcagaatccgctgcttacacagagccaggcgctccaccagattgtccacggcgatgttggagggggcgcagcacagaacctttaagccttgtttcacagcttgaagaatgatctcaaccacagtcgtggttttcccagtgccaggaggtccatggatgatggcaagttctttctgagacagcgcaaatgaaaccgcttctttctgggaggtgtccaggcaggtgttgaagaatgtcagcgggtgtatttcactggcaggactgggagcagatctgccaaagagcacttctatgagtgaggaggctgggccagaatgatacttctttagagcaatcagggcttttttcagtcgcctgtaagtgacatcattggcaagttttaacagtctgtaggaattctctcggtccaagctcaactggaaatcgtgggactcatcaaaggccaccgtgaccgacttctgggtgacccgggtcaagatcccagtggccagctgactgccctcattagcagcatcgtacaggcccacgatatcaccagaagtaaagctgttactgggaagagctgccgcggacccgtatcgcctgggctcaaaggtgaccagcagccgtccgtacagcccagtgcgctggctggatacctgcagcttcagcaaacacacgcctcggctctggagctctttcagagagatgttctcctgccaggacctgcgctcctccacctccgcgtctctctcgagctccagcaggtccagttgcttggtcacgaagctctccacagctgccgaggccattctagacctgcagggtttaaacgcgcgctagcggcgccgatatcttcgaaagatctggccgcctcggcccttaagaccggtggatcggccgcgggtacaattccgcagcttttagagcagaagtaacacttccgtacaggcctagaagtaaaggcaacatccactgaggagcagttctttgatttgcaccaccaccggatccgggacctgaaataaaagacaaaaaagactaaacttaccagttatctgcagaattcgcccttcgacgcgtctcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggcctcgagtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgaccagaggtaccgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 65 - DNA, 3459 bp, synthetic construct
  source 1..3459: mol_type = other DNA
atggcctcggcagctgtggagagcttcgtgaccaagcaactggacctgctggagctcgagagagacgcggaggtggaggagcgcaggtcctggcaggagaacatctctctgaaagagctccagagccgaggcgtgtgtttgctgaagctgcaggtatccagccagcgcactgggctgtacggacggctgctggtcacctttgagcccaggcgatacgggtccgcggcagctcttcccagtaacagctttacttctggtgatatcgtgggcctgtacgatgctgctaatgagggcagtcagctggccactgggatcttgacccgggtcacccagaagtcggtcacggtggcctttgatgagtcccacgatttccagttgagcttggaccgagagaattcctacagactgttaaaacttgccaatgatgtcacttacaggcgactgaaaaaagccctgattgctctaaagaagtatcattctggcccagcctcctcactcatagaagtgctctttggcagatctgctcccagtcctgccagtgaaatacacccgctgacattcttcaacacctgcctggacacctcccagaaagaagcggtttcatttgcgctgtctcagaaagaacttgccatcatccatggacctcctggcactgggaaaaccacgactgtggttgagatcattcttcaagctgtgaaacaaggcttaaaggttctgtgctgcgccccctccaacatcgccgtggacaatctggtggagcgcctggctctgtgtaagcagcggattctgcgcctgggacaccctgcccgcctcctggagtccattcagcagcactccctggatgcggttttagcgcggagcgacagtgcccagattgttgcagatatcaggaaggacatcgaccaggtctttgtgaaaaacaaaaagacccaggataagagagagaaaagtaattttcgaaatgaaattaagctgttaagaaaagaactgaaggagagggaagaagcagctatgctcgagagcctcacttcggcaaacgtggtccttgcaacaaacacaggtgcgtctgccgatggccccctgaagttgctgcccgagagctacttcgacgtggtggtcattgacgagtgtgcccaggccctcgaggcgagctgctggatccccctgctgaaggccagaaagtgcatcctggcgggcgatcacaagcagctgccccccaccacagtctctcacaaggctgcgctggcaggactgtcactcagcctgatggaacgcctggctgaggagtacggcgcgagggtggtgcggacactgacggtgcagtaccgcatgcaccaggctatcatgcgctgggcctcagacaccatgtaccttgggcagctcacagcccactcttccgtggcaaggcacctcctgagggacctcccaggtgtggctgccacagaagagacgggtgtgcccctgctcttggtggacaccgccggctgcgggctgtttgagctggaggaggaggacgaacagtcgaaagggaaccctggcgaagtccgcctcgtcagtttgcacatccaggctctggtggacgctggtgttccagcccgtgacattgctgtggtctcgccatacaacctccaggtggacctgctcagacagagccttgtgcacaggcaccctgagcttgaaatcaagtctgtcgatggcttccaaggccgagagaaggaggccgtgatactgtccttcgtcagatccaacaggaaaggtgaagttggttttcttgctgaggaccggaggatcaacgtggctgtcacccgtgcccgacgccacgtggcggtcatctgtgactcccgtactgtcaacaaccatgcatttttgaagaccctggtggagtatttcacacagcatggggaagtacgcacggcctttgagtatcttgacgatattgtcccagaaaactattcccatgagaactcccagggttccagccacgctgccaccaagccccagggacctgctgcgtccaccaggaccggaagccagcggcaggagggaggccaggaggctgcagcacctgccagacagggccggaagaagccggctgggaagtctctggcctctgaagctccatctcagcccagcctcaacggaggcagcccagagggagtggagagccaagatggcgtggaccacttccgggccatgatagtggagttcatggccagcaagaagatgcagttggagtttcctccttccctcaattcccacgacaggctgcgggtccaccaaatagccgaggagcacgggctgaggcacgacagttctggggaagggaagaggaggttcatcactgtgagcaagagggccccgcgaccccgagcagccctgggacccccagcagggaccggtggcccagcccctctccagccagtgccccctacccctgcgcagacagagcagcctcccagggagcagcgtggcccagaccagcctgatctgaggacgctgcacctggagagactgcagagggtcaggagcgcgcaggggcagcccgccagcaaggagcagcaggcctcagggcagcagaaacttccagaaaagaaaaagaaaaaagccaaaggacatccggccacagatctgcccacggaggaggactttgaggccctggtttctgccgccgttaaggctgataacacctgcggctttgccaagtgcacagccggcgtcacaaccctgggccagttctgccagctctgcagccgccgctactgcctcagccaccacctgcccgagatccatggctgcggtgagagggctcgcgcccatgcccggcagagaatcagccgggaaggggtcctctatgccggcagcgggaccaagaacggatccctggacccagccaagagggcccagctgcagaggaggctggataagaagctgagtgagctcagcaaccagaggaccagccggaggaaggagagggggacgctcgagggaagcggagctactaacttcagcctgctgaagcaggctggagacgtggaggagaaccctggacctatggcttgtggtctggtcgccagcaacctgaatctcaaacctggagagtgccttcgagtgcgaggcgaggtggctcctgacgctaagagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgtgcatcaccttcgaccaggccaacctgaccgtcaagctgccagatggatacgaattcaagttccccaaccgcctcaacctggaggccatcaactacatggcagctgacggtgacttcaagatcaaatgtgtggcctttgactga

SEQ ID NO: 66 - protein, 1152 aa, synthetic construct
MASAAVESFVTKQLDLLELERDAEVEERRSWQENISLKELQSRGVCLLKLQVSSQRTGLYGRLLVTFEPRRYGSAAALPSNSFTSGDIVGLYDAANEGSQLATGILTRVTQKSVTVAFDESHDFQLSLDRENSYRLLKLANDVTYRRLKKALIALKKYHSGPASSLIEVLFGRSAPSPASEIHPLTFFNTCLDTSQKEAVSFALSQKELAIIHGPPGTGKTTTVVEIILQAVKQGLKVLCCAPSNIAVDNLVERLALCKQRILRLGHPARLLESIQQHSLDAVLARSDSAQIVADIRKDIDQVFVKNKKTQDKREKSNFRNEIKLLRKELKEREEAAMLESLTSANVVLATNTGASADGPLKLLPESYFDVVVIDECAQALEASCWIPLLKARKCILAGDHKQLPPTTVSHKAALAGLSLSLMERLAEEYGARVVRTLTVQYRMHQAIMRWASDTMYLGQLTAHSSVARHLLRDLPGVAATEETGVPLLLVDTAGCGLFELEEEDEQSKGNPGEVRLVSLHIQALVDAGVPARDIAVVSPYNLQVDLLRQSLVHRHPELEIKSVDGFQGREKEAVILSFVRSNRKGEVGFLAEDRRINVAVTRARRHVAVICDSRTVNNHAFLKTLVEYFTQHGEVRTAFEYLDDIVPENYSHENSQGSSHAATKPQGPAASTRTGSQRQEGGQEAAAPARQGRKKPAGKSLASEAPSQPSLNGGSPEGVESQDGVDHFRAMIVEFMASKKMQLEFPPSLNSHDRLRVHQIAEEHGLRHDSSGEGKRRFITVSKRAPRPRAALGPPAGTGGPAPLQPVPPTPAQTEQPPREQRGPDQPDLRTLHLERLQRVRSAQGQPASKEQQASGQQKLPEKKKKKAKGHPATDLPTEEDFEALVSAAVKADNTCGFAKCTAGVTTLGQFCQLCSRRYCLSHHLPEIHGCGERARAHARQRISREGVLYAGSGTKNGSLDPAKRAQLQRRLDKKLSELSNQRTSRRKERGTLEGSGATNFSLLKQAGDVEENPGPMACGLVASNLNLKPGECLRVRGEVAPDAKSFVLNLGKDSNNLCLHFNPRFNAHGDANTIVCNSKDGGAWGTEQREAVFPFQPGSVAEVCITFDQANLTVKLPDGYEFKFPNRLNLEAINYMAADGDFKIKCVAFD

SEQ ID NO: 67 - DNA, 6278 bp, synthetic construct
  source 1..6278: mol_type = other DNA
  unsure 980..1085: note = mITR
  unsure 1136..1684: note = 546 promoter
  unsure 1714..1812: note = Mod SV40 Intron
  unsure 1838..2803: note = PGAP3
  unsure 2804..2869: note = P2A
  unsure 2876..3283: note = hGal1
  unsure 3312..3458: note = BGHpA
  unsure 3483..3623: note = ITR
  unsure 4526..5335: note = KanR
  unsure 5489..6110: note = Ori
gcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagattcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtaccgaattcacgcgtgaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaacgcgtcgaagggcgaattctgcagataactggtaagtttagtctttttgtcttttatttcaggtcccggatccggtggtggtgcaaatcaaagaactgctcctcagtggatgttgcctttacttctaggcctgtacggaagtgttactaccggtatggccggcctggcggcgcggttggtcctgctagctggggcagcggcgctggcgagcggctcccagggcgaccgtgagccggtgtaccgcgactgcgtactgcagtgcgaagagcagaactgctctgggggcgctctgaatcacttccgctcccgccagccaatctacatgagtctagcaggctggacctgtcgggacgactgtaagtatgagtgtatgtgggtcaccgttgggctctacctccaggaaggtcacaaagtgcctcagttccatggcaagtggcccttctcccggttcctgttctttcaagagccggcatcggccgtggcctcgtttctcaatggcctggccagcctggtgatgctctgccgctaccgcaccttcgtgccagcctcctcccccatgtaccacacctgtgtggccttcgcctgggtgtccctcaatgcatggttctggtccacagttttccacaccagggacactgacctcacagagaaaatggactacttctgtgcctccactgtcatcctacactcaatctacctgtgctgcgtcaggaccgtggggctgcagcacccagctgtggtcagtgccttccgggctctcctgctgctcatgctgaccgtgcacgtctcctacctgagcctcatccgcttcgactatggctacaacctggtggccaacgtggctattggcctggtcaacgtggtgtggtggctggcctggtgcctgtggaaccagcggcggctgcctcacgtgcgcaagtgcgtggtggtggtcttgctgctgcaggggctgtccctgctcgagctgcttgacttcccaccgctcttctgggtcctggatgcccatgccatctggcacatcagcaccatccctgtccacgtcctctttttcagctttctggaagatgacagcctgtacctgctgaaggaatcagaggacaagttcaagctggacctcgagggaagcggagctactaacttcagcctgctgaagcaggctggagacgtggaggagaaccctggaccttctagaatggcttgtggtctggtcgccagcaacctgaatctcaaacctggagagtgccttcgagtgcgaggcgaggtggctcctgacgctaagagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgtgcatcaccttcgaccaggccaacctgaccgtcaagctgccagatggatacgaattcaagttccccaaccgcctcaacctggaggccatcaactacatggcagctgacggtgacttcaagatcaaatgtgtggcctttgactgatctagaactagtggcgcgccggccggccgaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgagggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 68 - DNA, 6283 bp, synthetic construct
  source 1..6283: mol_type = other DNA
  unsure 980..1085: note = mITR
  unsure 1132..1411: note = CMV Enhancer
  unsure 1418..1687: note = CBA promoter
  unsure 1719..1817: note = Mod SV40 Intron
  unsure 1843..2808: note = PGAP3
  unsure 2809..2874: note = P2A
  unsure 2881..3288: note = hGal1
  unsure 3317..3463: note = BGHpA
  unsure 3488..3628: note = ITR
  unsure 4531..5340: note = KanR
  unsure 5494..6115: note = Ori
gcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagattcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagacgcgtcgaagggcgaattctgcagataactggtaagtttagtctttttgtcttttatttcaggtcccggatccggtggtggtgcaaatcaaagaactgctcctcagtggatgttgcctttacttctaggcctgtacggaagtgttactaccggtatggccggcctggcggcgcggttggtcctgctagctggggcagcggcgctggcgagcggctcccagggcgaccgtgagccggtgtaccgcgactgcgtactgcagtgcgaagagcagaactgctctgggggcgctctgaatcacttccgctcccgccagccaatctacatgagtctagcaggctggacctgtcgggacgactgtaagtatgagtgtatgtgggtcaccgttgggctctacctccaggaaggtcacaaagtgcctcagttccatggcaagtggcccttctcccggttcctgttctttcaagagccggcatcggccgtggcctcgtttctcaatggcctggccagcctggtgatgctctgccgctaccgcaccttcgtgccagcctcctcccccatgtaccacacctgtgtggccttcgcctgggtgtccctcaatgcatggttctggtccacagttttccacaccagggacactgacctcacagagaaaatggactacttctgtgcctccactgtcatcctacactcaatctacctgtgctgcgtcaggaccgtggggctgcagcacccagctgtggtcagtgccttccgggctctcctgctgctcatgctgaccgtgcacgtctcctacctgagcctcatccgcttcgactatggctacaacctggtggccaacgtggctattggcctggtcaacgtggtgtggtggctggcctggtgcctgtggaaccagcggcggctgcctcacgtgcgcaagtgcgtggtggtggtcttgctgctgcaggggctgtccctgctcgagctgcttgacttcccaccgctcttctgggtcctggatgcccatgccatctggcacatcagcaccatccctgtccacgtcctctttttcagctttctggaagatgacagcctgtacctgctgaaggaatcagaggacaagttcaagctggacctcgagggaagcggagctactaacttcagcctgctgaagcaggctggagacgtggaggagaaccctggaccttctagaatggcttgtggtctggtcgccagcaacctgaatctcaaacctggagagtgccttcgagtgcgaggcgaggtggctcctgacgctaagagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgtgcatcaccttcgaccaggccaacctgaccgtcaagctgccagatggatacgaattcaagttccccaaccgcctcaacctggaggccatcaactacatggcagctgacggtgacttcaagatcaaatgtgtggcctttgactgatctagaactagtggcgcgccggccggccgaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgagggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 69 - DNA, 1446 bp, synthetic construct
  source 1..1446: mol_type = other DNA
atggccggcctggcggcgcggttggtcctgctagctggggcagcggcgctggcgagcggctcccagggcgaccgtgagccggtgtaccgcgactgcgtactgcagtgcgaagagcagaactgctctgggggcgctctgaatcacttccgctcccgccagccaatctacatgagtctagcaggctggacctgtcgggacgactgtaagtatgagtgtatgtgggtcaccgttgggctctacctccaggaaggtcacaaagtgcctcagttccatggcaagtggcccttctcccggttcctgttctttcaagagccggcatcggccgtggcctcgtttctcaatggcctggccagcctggtgatgctctgccgctaccgcaccttcgtgccagcctcctcccccatgtaccacacctgtgtggccttcgcctgggtgtccctcaatgcatggttctggtccacagttttccacaccagggacactgacctcacagagaaaatggactacttctgtgcctccactgtcatcctacactcaatctacctgtgctgcgtcaggaccgtggggctgcagcacccagctgtggtcagtgccttccgggctctcctgctgctcatgctgaccgtgcacgtctcctacctgagcctcatccgcttcgactatggctacaacctggtggccaacgtggctattggcctggtcaacgtggtgtggtggctggcctggtgcctgtggaaccagcggcggctgcctcacgtgcgcaagtgcgtggtggtggtcttgctgctgcaggggctgtccctgctcgagctgcttgacttcccaccgctcttctgggtcctggatgcccatgccatctggcacatcagcaccatccctgtccacgtcctctttttcagctttctggaagatgacagcctgtacctgctgaaggaatcagaggacaagttcaagctggacctcgagggaagcggagctactaacttcagcctgctgaagcaggctggagacgtggaggagaaccctggaccttctagaatggcttgtggtctggtcgccagcaacctgaatctcaaacctggagagtgccttcgagtgcgaggcgaggtggctcctgacgctaagagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgtgcatcaccttcgaccaggccaacctgaccgtcaagctgccagatggatacgaattcaagttccccaaccgcctcaacctggaggccatcaactacatggcagctgacggtgacttcaagatcaaatgtgtggcctttgactga

SEQ ID NO: 70 - protein, 481 aa, synthetic construct
MAGLAARLVLLAGAAALASGSQGDREPVYRDCVLQCEEQNCSGGALNHFRSRQPIYMSLAGWTCRDDCKYECMWVTVGLYLQEGHKVPQFHGKWPFSRFLFFQEPASAVASFLNGLASLVMLCRYRTFVPASSPMYHTCVAFAWVSLNAWFWSTVFHTRDTDLTEKMDYFCASTVILHSIYLCCVRTVGLQHPAVVSAFRALLLLMLTVHVSYLSLIRFDYGYNLVANVAIGLVNVVWWLAWCLWNQRRLPHVRKCVVVVLLLQGLSLLELLDFPPLFWVLDAHAIWHISTIPVHVLFFSFLEDDSLYLLKESEDKFKLDLEGSGATNFSLLKQAGDVEENPGPSRMACGLVASNLNLKPGECLRVRGEVAPDAKSFVLNLGKDSNNLCLHFNPRFNAHGDANTIVCNSKDGGAWGTEQREAVFPFQPGSVAEVCITFDQANLTVKLPDGYEFKFPNRLNLEAINYMAADGDFKIKCVAFD

SEQ ID NO: 71 - DNA, 6032 bp, synthetic construct
  source 1..6032: mol_type = other DNA
  unsure 980..1085: note = mITR
  unsure complement(1220..1284): note = SOD shRNA
  unsure complement(1285..1383): note = H1 Promoter
  unsure 1543..1822: note = CMV enhancer
  unsure 1829..2098: note = B-Actin promoter
  unsure 2163..2261: note = modSV40_late_16s_int
  unsure 2409..2813: note = msGalectin1
  unsure 2820..2885: note = P2A
  unsure 2886..3071: note = MT1X
  unsure 3116..3262: note = BGHpA
  unsure 3342..3482: note = ITR
  unsure 4280..5089: note = Kan R
  unsure 5244..5863: note = Ori
gcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcaattcacgcgccggtaccgaattcacgcgtggtacctacactttatgcttccggctcgtatgttgtgtggaattgtgagcggataacaatttcacacaggaaacagctatgaccatgattacgccaagctttccaaaaaagcatggattccatgttcatgatctcttgaatcatgaacatggaatccatggatccgagtggtctcatacagaacttataagattcccaaatccaaagacatttcacgtttatggtgatttcccagaacacatagcgacatgcaaatatgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggttttaagggccgaggcggccagatctttcgaagatatcggcgccgctagcgcggccgcgtttaaaccctgcaggtctagaatcatggcctgtggtctggtcgccagcaacctgaatctcaaacctggggaatgtctcaaagttcggggagaggtggcctcggacgccaagagctttgtgctgaacctgggaaaagacagcaacaacctgtgcctacacttcaatcctcgcttcaatgcccatggagacgccaacaccattgtgtgtaacaccaaggaagatgggacctggggaaccgaacaccgggaacctgccttccccttccagcccgggagcatcacagaggtgtgcatcacctttgaccaggctgacctgaccatcaagctgccagacggacatgaattcaagttccccaaccgcctcaacatggaggccatcaactacatggcggcggatggagacttcaagattaagtgcgtggcctttgagtctagaggaagcggagctactaacttcagcctgctgaagcaggctggagacgtggaggagaaccctggacctatggaccccaactgctcctgctcgcctgttggctcctgtgcctgtgccggctcctgcaaatgcaaagagtgcaaatgcacctcctgcaagaagagctgctgctcctgctgccctgtgggctgtgccaagtgtgcccagggctgcatctgcaaagggacgtcagacaagtgcagctgctgtgcctgaactagtaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 72 - DNA, 663 bp, synthetic construct
  source 1..663: mol_type = other DNA
atggcctgtggtctggtcgccagcaacctgaatctcaaacctggggaatgtctcaaagttcggggagaggtggcctcggacgccaagagctttgtgctgaacctgggaaaagacagcaacaacctgtgcctacacttcaatcctcgcttcaatgcccatggagacgccaacaccattgtgtgtaacaccaaggaagatgggacctggggaaccgaacaccgggaacctgccttccccttccagcccgggagcatcacagaggtgtgcatcacctttgaccaggctgacctgaccatcaagctgccagacggacatgaattcaagttccccaaccgcctcaacatggaggccatcaactacatggcggcggatggagacttcaagattaagtgcgtggcctttgagtctagaggaagcggagctactaacttcagcctgctgaagcaggctggagacgtggaggagaaccctggacctatggaccccaactgctcctgctcgcctgttggctcctgtgcctgtgccggctcctgcaaatgcaaagagtgcaaatgcacctcctgcaagaagagctgctgctcctgctgccctgtgggctgtgccaagtgtgcccagggctgcatctgcaaagggacgtcagacaagtgcagctgctgtgcctga

SEQ ID NO: 73 - protein, 220 aa, synthetic construct
MACGLVASNLNLKPGECLKVRGEVASDAKSFVLNLGKDSNNLCLHFNPRFNAHGDANTIVCNTKEDGTWGTEHREPAFPFQPGSITEVCITFDQADLTIKLPDGHEFKFPNRLNMEAINYMAADGDFKIKCVAFESRGSGATNFSLLKQAGDVEENPGPMDPNCSCSPVGSCACAGSCKCKECKCTSCKKSCCSCCPVGCAKCAQGCICKGTSDKCSCCA

SEQ ID NO: 74 - DNA, 6035 bp, synthetic construct
  source 1..6035: mol_type = other DNA
  unsure 980..1085: note = mITR
  unsure complement(1220..1284): note = SOD shRNA
  unsure complement(1285..1383): note = H1 Promoter
  unsure 1543..1822: note = CMV enhancer
  unsure 1829..2098: note = Chicken B-Actin promoter
  unsure 2163..2261: note = modSV40_late_16s_int
  unsure 2402..2811: note = hGalectin1
  unsure 2818..2833: note = P2A
  unsure 2884..3069: note = MT1X
  unsure 3119..3265: note = BGHpA
  unsure 3345..3485: note = ITR
  unsure 4283..5092: note = Kan R
  unsure 5247..5866: note = Ori
gcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcaattcacgcgccggtaccgaattcacgcgtggtacctacactttatgcttccggctcgtatgttgtgtggaattgtgagcggataacaatttcacacaggaaacagctatgaccatgattacgccaagctttccaaaaaagcatggattccatgttcatgatctcttgaatcatgaacatggaatccatggatccgagtggtctcatacagaacttataagattcccaaatccaaagacatttcacgtttatggtgatttcccagaacacatagcgacatgcaaatatgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggttttaagggccgaggcggccagatctttcgaagatatcggcgccgctagcgcggccgcgtttaaaccctgcaggttctagaatggcttgtggtctggtcgccagcaacctgaatctcaaacctggagagtgccttcgagtgcgaggcgaggtggctcctgacgctaagagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgtgcatcaccttcgaccaggccaacctgaccgtcaagctgccagatggatacgaattcaagttccccaaccgcctcaacctggaggccatcaactacatggcagctgacggtgacttcaagatcaaatgtgtggcctttgactctagaggaagcggagctactaacttcagcctgctgaagcaggctggagacgtggaggagaaccctggacctatggaccccaactgctcctgctcgcctgttggctcctgtgcctgtgccggctcctgcaaatgcaaagagtgcaaatgcacctcctgcaagaagagctgctgctcctgctgccctgtgggctgtgccaagtgtgcccagggctgcatctgcaaagggacgtcagacaagtgcagctgctgtgcctgaactagtctagaaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 75 - DNA, 663 bp, synthetic construct
  source 1..663: mol_type = other DNA
atggcttgtggtctggtcgccagcaacctgaatctcaaacctggagagtgccttcgagtgcgaggcgaggtggctcctgacgctaagagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgtgcatcaccttcgaccaggccaacctgaccgtcaagctgccagatggatacgaattcaagttccccaaccgcctcaacctggaggccatcaactacatggcagctgacggtgacttcaagatcaaatgtgtggcctttgactctagaggaagcggagctactaacttcagcctgctgaagcaggctggagacgtggaggagaaccctggacctatggaccccaactgctcctgctcgcctgttggctcctgtgcctgtgccggctcctgcaaatgcaaagagtgcaaatgcacctcctgcaagaagagctgctgctcctgctgccctgtgggctgtgccaagtgtgcccagggctgcatctgcaaagggacgtcagacaagtgcagctgctgtgcctga

SEQ ID NO: 76 - protein, 220 aa, synthetic construct
MACGLVASNLNLKPGECLRVRGEVAPDAKSFVLNLGKDSNNLCLHFNPRFNAHGDANTIVCNSKDGGAWGTEQREAVFPFQPGSVAEVCITFDQANLTVKLPDGYEFKFPNRLNLEAINYMAADGDFKIKCVAFDSRGSGATNFSLLKQAGDVEENPGPMDPNCSCSPVGSCACAGSCKCKECKCTSCKKSCCSCCPVGCAKCAQGCICKGTSDKCSCCA

SEQ ID NO: 77 - DNA, 49 bp, synthetic construct
  source 1..49: mol_type = other DNA
aataaaagatctttattttcattagatctgtgtgttggttttttgtgtg

SEQ ID NO: 78 - DNA, 5433 bp, synthetic construct
  source 1..5433: mol_type = other DNA
  unsure 980..1085: note = mITR
  unsure 1136..1684: note = 546 promoter
  unsure 1714..1812: note = Mod SV40 Intron
  unsure 1844..2026: note = MT1X
  unsure 2027..2092: note = P2A
  unsure 2099..2281: note = MT1E
  unsure 2282..2347: note = P2A
  unsure 2354..2539: note = MT2A
  unsure 2568..2616: note = synthetic pA
  unsure 2568..2616: note = synthetic pA
  unsure 2641..2781: note = ITR
  unsure 3681..4490: note = KanR
  unsure 4644..5265: note = Ori
gcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagattcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtaccgaattcacgcgtgaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaacgcgtcgaagggcgaattctgcagataactggtaagtttagtctttttgtcttttatttcaggtcccggatccggtggtggtgcaaatcaaagaactgctcctcagtggatgttgcctttacttctaggcctgtacggaagtgttactaccggtagtactatggaccccaactgctcctgctcgcctgttggctcctgtgcctgtgccggctcctgcaaatgcaaagagtgcaaatgcacctcctgcaagaagagctgctgctcctgctgccctgtgggctgtgccaagtgtgcccagggctgcatctgcaaagggacgtcagacaagtgcagctgctgtgccggaagcggagctactaacttcagcctgctgaagcaggctggagacgtggaggagaaccctggacctgatatcatggaccccaactgctcttgcgccactggtggctcctgcacgtgcgccggctcctgcaagtgcaaagagtgcaaatgcacctcctgcaagaagagctgctgttcctgctgccccgtgggctgtgccaagtgtgcccagggctgcgtctgcaaaggggcatcggagaagtgcagctgctgtgccggaagcggagctactaacttcagcctgctgaagcaggctggagacgtggaggagaaccctggacctgttaacatggatcccaactgctcctgcgccgccggtgactcctgcacctgcgccggctcctgcaaatgcaaagagtgcaaatgcacctcctgcaagaaaagctgctgctcctgctgccctgtgggctgtgccaagtgtgcccagggctgcatctgcaaaggggcgtcggacaagtgcagctgctgcgcctgatctagaactagtggcgcgccggccggccaataaaagatctttattttcattagatctgtgtgttggttttttgtgtggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 79 - DNA, 5438 bp, synthetic construct
  source 1..5438: mol_type = other DNA
  unsure 980..1085: note = mITR
  unsure 1132..1411: note = CMV Enhancer
  unsure 1418..1687: note = CBA Promoter
  unsure 1719..1817: note = Mod SV40 Intron
  unsure 1849..2031: note = MT1X
  unsure 2032..2097: note = P2A
  unsure 2104..2286: note = MT1E
  unsure 2287..2352: note = P2A
  unsure 2359..2544: note = MT2A
  unsure 2573..2621: note = synthetic pA
  unsure 2646..2786: note = ITR
  unsure 3686..4495: note = KanR
  unsure 4649..5270: note = Ori
gcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagattcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagacgcgtcgaagggcgaattctgcagataactggtaagtttagtctttttgtcttttatttcaggtcccggatccggtggtggtgcaaatcaaagaactgctcctcagtggatgttgcctttacttctaggcctgtacggaagtgttactaccggtagtactatggaccccaactgctcctgctcgcctgttggctcctgtgcctgtgccggctcctgcaaatgcaaagagtgcaaatgcacctcctgcaagaagagctgctgctcctgctgccctgtgggctgtgccaagtgtgcccagggctgcatctgcaaagggacgtcagacaagtgcagctgctgtgccggaagcggagctactaacttcagcctgctgaagcaggctggagacgtggaggagaaccctggacctgatatcatggaccccaactgctcttgcgccactggtggctcctgcacgtgcgccggctcctgcaagtgcaaagagtgcaaatgcacctcctgcaagaagagctgctgttcctgctgccccgtgggctgtgccaagtgtgcccagggctgcgtctgcaaaggggcatcggagaagtgcagctgctgtgccggaagcggagctactaacttcagcctgctgaagcaggctggagacgtggaggagaaccctggacctgttaacatggatcccaactgctcctgcgccgccggtgactcctgcacctgcgccggctcctgcaaatgcaaagagtgcaaatgcacctcctgcaagaaaagctgctgctcctgctgccctgtgggctgtgccaagtgtgcccagggctgcatctgcaaaggggcgtcggacaagtgcagctgctgcgcctgatctagaactagtggcgcgccggccggccaataaaagatctttattttcattagatctgtgtgttggttttttgtgtggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 80 - DNA, 696 bp, synthetic construct
  source 1..696: mol_type = other DNA
atggaccccaactgctcctgctcgcctgttggctcctgtgcctgtgccggctcctgcaaatgcaaagagtgcaaatgcacctcctgcaagaagagctgctgctcctgctgccctgtgggctgtgccaagtgtgcccagggctgcatctgcaaagggacgtcagacaagtgcagctgctgtgccggaagcggagctactaacttcagcctgctgaagcaggctggagacgtggaggagaaccctggacctgatatcatggaccccaactgctcttgcgccactggtggctcctgcacgtgcgccggctcctgcaagtgcaaagagtgcaaatgcacctcctgcaagaagagctgctgttcctgctgccccgtgggctgtgccaagtgtgcccagggctgcgtctgcaaaggggcatcggagaagtgcagctgctgtgccggaagcggagctactaacttcagcctgctgaagcaggctggagacgtggaggagaaccctggacctgttaacatggatcccaactgctcctgcgccgccggtgactcctgcacctgcgccggctcctgcaaatgcaaagagtgcaaatgcacctcctgcaagaaaagctgctgctcctgctgccctgtgggctgtgccaagtgtgcccagggctgcatctgcaaaggggcgtcggacaagtgcagctgctgcgcctga

SEQ ID NO: 81 - protein, 231 aa, synthetic construct
MDPNCSCSPVGSCACAGSCKCKECKCTSCKKSCCSCCPVGCAKCAQGCICKGTSDKCSCCAGSGATNFSLLKQAGDVEENPGPDIMDPNCSCATGGSCTCAGSCKCKECKCTSCKKSCCSCCPVGCAKCAQGCVCKGASEKCSCCAGSGATNFSLLKQAGDVEENPGPVNMDPNCSCAAGDSCTCAGSCKCKECKCTSCKKSCCSCCPVGCAKCAQGCICKGASDKCSCCA